(19) United States
(12) Patent Application Publication
(10) Pub. No.: US 2024/0092818 A1
(43) Pub. Date: Mar. 21, 2024

(54) SYNTHESIS OF ANTIVIRAL NUCLEOSIDES

(71) Applicant: Merck Sharp & Dohme LLC, Rahway, NJ (US)

(72) Inventors: Tamas Benkovics, Louisville, CO (US); Patrick S. Fier, Monroe Township, NJ (US); Anna Fryszkowska, New York, NY (US); Mark A. Huffman, Warren, NJ (US); Tetsuji Itoh, Somerset, NJ (US); Jongrock Kong, Princeton, NJ (US); Peter E. Maligres, Fanwood, NJ (US); Kevin M. Maloney, Metuchen, NJ (US); John McIntosh, Brookline, MA (US); Grant S. Murphy, Princeton, NJ (US); Steven M. Silverman, Jersey City, NJ (US); Hao Yang, Scotch Plains, NJ (US)

(73) Assignee: Merck Sharp & Dohme LLC, Rahway, NJ (US)

(21) Appl. No.: 18/256,058

(22) PCT Filed: Dec. 17, 2021
(86) PCT No.: PCT/US2021/064021

§ 371 Date: Jun. 6, 2023

Related U.S. Application Data
(60) Provisional application No. 63/192,912, filed on May 25, 2021.
(60) Provisional application No. 63/182,171, filed on Apr. 30, 2021.
(60) Provisional application No. 63/127,484, filed on Dec. 18, 2020.

(51) Int. Cl. C07H 17/02; C07H 15/04

(52) U.S. Cl. CPC C07H 17/02; C07H 15/04

(57) ABSTRACT

The present invention relates to efficient synthetic processes useful in the preparation of antiviral nucleosides, particularly uridine 4-oxime 5′-(2-methylpropanoate) {(2R,3S,4R,5R)-3,4-dihydroxy-5-[4-(hydroxyimino)-2-oxo-3,4-dihydropyrimidin-1(2H)-yl]oxolan-2-yl}methyl 2-methylpropanoate and pharmaceutically acceptable salts, derivatives, tautomers, isomers, and prodrugs of, which may be active as antiviral agents, as well as compositions and methods thereof. The present invention also encompasses intermediates useful in the disclosed synthetic processes and the methods of their preparation.

### SEQUENCE LISTING

The instant application contains a Sequence Listing which has been submitted electronically in ASCII format and is hereby incorporated by reference in its entirety. Said ASCII copy, created on Dec. 13, 2021, is named 25183-WO-PCT_SL.txt and is 155,715 bytes in size.

### FIELD OF THE INVENTION

The present invention relates to efficient synthetic processes useful in the preparation of nucleosides, particularly uridine 4-oxime 5′-(2-methylpropanoate) {(2R,3S,4R,5R)-3,4-dihydroxy-5-[4-(hydroxyimino)-2-oxo-3,4-dihydropyrimidin-1(2H)-yl]oxolan-2-yl}methyl 2-methylpropanoate and pharmaceutically acceptable salts, derivatives, tautomers, isomers, and prodrugs thereof, which may be active as antiviral agents. The present invention also encompasses intermediates useful in the disclosed synthetic processes and the methods of their preparation.

### BACKGROUND OF THE INVENTION

Viral infections, such as infections caused by Eastern Equine Encephalitis Virus (EEEV), Western Equine Encephalitis Virus (WEEV), and Venezuelan Equine Encephalitis Virus (VEEV), Chikungunya fever virus (CHIK), Ebola virus, influenza virus, respiratory syncytial virus (RSV), Zika virus, and coronaviruses, such as Severe Acute Respiratory Syndrome Coronavirus (SARS-CoV), Middle East Respiratory Syndrome Coronavirus (MERS-CoV), and, most recently, SARS-CoV-2 (also known as 2019-nCoV), continue to cause illnesses, both mild and severe to life-threatening and fatal, across the globe.

EEEV, WEEV, VEEV, and CHIK virus are vector-borne viruses (family Togaviridae, genus Alphavirus) that can be transmitted to humans through mosquito bites. The equine encephalitis viruses are CDC Category B pathogens, and the CHIK virus is Category C.

Coronaviruses cause a large percentage of respiratory illness in humans, which can be severe or life-threatening. SARS-CoV-1, which emerged in 2002, has caused at least 8439 human illnesses globally and at least 812 deaths (WHO Cumulative Number of Reported Probable Cases of SARS, From 1 Nov. 2002 To 4 Jul. 2003, downloaded from https://www.who.int/csr/sars/country/2003_07_04/en/, on Aug. 12, 2020). Similarly, MERS-CoV emerged in 2012 and has caused at least 2519 human illnesses globally and at least 866 deaths (WHO Middle East respiratory syndrome, MERS situation update, January 2020, downloaded from http://www.emro.who.int/health-topics/mers-cov/mers-outbreaks.html, on Aug. 12, 2020). More recently, SARS-CoV-2 emerged in 2019, and it has caused at least 237,655,302 human illnesses globally and at least 4,846,981 deaths (COVID-19 Weekly Operational Update, Issue No. 75, published 12 Oct. 2021, downloaded from https://www.who.int/publications/m/item/weekly-operational-update-on-covid-19---12-october-2021, on Oct. 19, 2021). SARS-CoV-2 causes disease referred to a COVID-19, which can include severe respiratory disease in humans and appears to also cause neurological disease and complications that include headache, dizziness, hypogeusia, neuralgia, encephalopathy, acute cerebrovascular diseases, impaired consciousness and skeletal muscular injury (Imran Ahmad and Farooq Azam Rathore, Neurological manifestations and complications of COVID-19: A literature review, 77 J. CLIN. NEUROSCI. 8-12 (2020)). Additional studies are needed to further characterize the SARS-CoV-2 virus and to identify ways to prevent and treat the COVID-19 disease, as well as diseases caused by other human coronaviruses.

β-D-N(4)-hydroxycytidine (NHC, 1-((2R,3R,4S,5R)-3,4-dihydroxy-5-(hydroxymethyl)tetrahydrofuran-2-yl)-4-(hydroxyamino)pyrimidin-2(1H)-one) was found to have antipestivirus and antihepacivirus activities. ANTIMICROB AGENTS CHEMOTHER, 2003, 47(1):244-54. β-D-N(4)-hydroxycytidine,

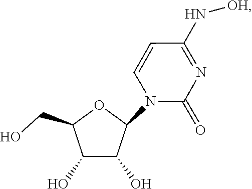 [SMILES: O=c1nc(NO)ccn1[C@@H]1O[C@H](CO)[C@@H](O)[C@H]1O]

derivatives, and methods for making the same are illustrated in PCT International Patent Application No. PCT/US2015/066144, which published as WO2016/106050, PCT International Application No. PCT/US2017/021759, which published as WO2017/156380, and PCT International Patent Application No. PCT/US2018/064503, which published as PCT International Patent Application Publication No. WO2019/113462, which are incorporated herein by reference in their entirety.

However, NHC has demonstrated apparent low oral bioavailability in cynomolgus macaques, and certain prodrugs were shown to improve oral bioavailability. See PCT International Patent Application No. PCT/US2018/064503, which published as PCT International Patent Application Publication No. WO2019/113462. These prodrugs include the uridine 4-oxime 5′-(2-methylpropanoate) {(2R,3S,4R,5R)-3,4-dihydroxy-5-[4-(hydroxyimino)-2-oxo-3,4-dihydropyrimidin-1(2H)-yl]oxolan-2-yl}methyl 2-methylpropanoate (Compound B)

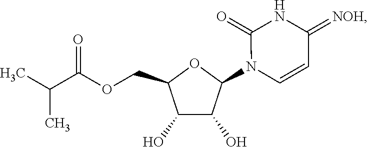

and pharmaceutically acceptable salts, derivatives, tautomers, isomers, and prodrugs of Compound B, as well as particular forms as disclosed in PCT International Patent Application No. PCT/US2021/048054, filed Aug. 27, 2021, which is incorporated herein by reference in its entirety. However, there is a need for synthetic routes that are efficient and employ green chemistry.

### SUMMARY OF THE INVENTION

The present invention relates to processes useful in the synthesis of nucleosides, particularly the antiviral nucleoside uridine 4-oxime 5′-(2-methylpropanoate) {(2R,3S,4R,5R)-3,4-dihydroxy-5-[4-(hydroxyimino)-2-oxo-3,4-dihydropyrimidin-1(2H)-yl]oxolan-2-yl}methyl 2-methylpropanoate (Compound B)

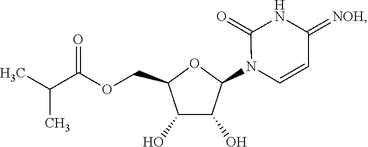

and pharmaceutically acceptable salts, derivatives, tautomers, isomers, and prodrugs of Compound B, as well as particular forms as disclosed in PCT International Patent Application No. PCT/US2021/048054, filed Aug. 27, 2021. The present invention encompasses processes that afford intermediates useful in the production of such antiviral nucleosides. The processes of the present invention afford advantages over previously known procedures and include a convergent, more efficient route to such antiviral nucleosides.

Other embodiments, aspects and features of the present disclosure are either further described in or will be apparent from the ensuing description, examples and appended claims.

### DETAILED DESCRIPTION OF THE INVENTION

### Abbreviations

ATP Adenosine 5′-triphosphate
DME 1,2-Dimethoxyethane
EtOAc Ethyl acetate
g/L Grams per liter
h hour
HMDS Bis(trimethylsilyl)amine, also known as hexamethyldisilazane
HPLC High-performance liquid chromatography
K₂HPO₄ Dipotassium phosphate, dipotassium hydrogen orthophosphate
KOH Potassium hydroxide
MeTHF Methyl tetrahydrofuran
mg Milligram
MgCl₂ Magnesium chloride
ml, mL Milliliter
mmol millimole
MnCl₂ Manganese chloride
MTBE Methyl tert-butyl ether
N Normality, number of mole equivalents per liter of solution
RPM Revolutions per minute
TEoA Triethanolamine
wt % Weight percent
μl, ul Microliter
μmol Micromole

Additional abbreviations may be defined throughout this disclosure.

### Definitions

Certain technical and scientific terms are specifically defined below. Unless specifically defined elsewhere in this document, all other technical and scientific terms used herein have the meaning commonly understood by one of ordinary skill in the art to which this disclosure relates. That is, terms used herein have their ordinary meaning, which is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc.

As used herein, and throughout this disclosure, the following terms, unless otherwise indicated, shall be understood to have the following meanings:

As used herein, including the appended claims, the singular forms of words, such as “a,” “an,” and “the,” include their corresponding plural references unless the context clearly dictates otherwise. In particular, “a,” “an,” and “the” item each include a single item selected from a list as well as mixtures of two or more items selected from the list.

As used herein, the terms “at least one” item or “one or more” item each include a single item selected from the list as well as mixtures of two or more items selected from the list. For example, “at least one S-methyl-5-thioribose kinase enzyme” (alternatively referred to as “S-methyl-5-thioribose kinase enzymes,” “at least one MTR kinase,” “MTR kinases,” “at least one MTR kinase enzyme,” or “MTR kinase enzymes”) refers to a single MTR kinase as well as to mixtures of two or more different MTR kinases. Similarly, the terms “at least two” items and “two or more” items each include mixtures of two items selected from the list as well as mixtures of three or more items selected from the list.

As used herein, the term “COVID-19” refers to the disease caused by SARS-CoV-2 infection. Subjects infected with SARS-CoV-2 who have developed symptoms are considered to have COVID-19.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition.

Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting. It is understood that wherever embodiments are described herein with the language “comprising,” otherwise analogous embodiments described in terms of “consisting of” and/or “consisting essentially of” are also provided.

Unless expressly stated to the contrary, all ranges cited herein are inclusive; i.e., the range includes the values for the upper and lower limits of the range as well as all values in between. All ranges also are intended to include all included sub-ranges, although not necessarily explicitly set forth. As an example, temperature ranges, percentages, ranges of equivalents, and the like described herein include the upper and lower limits of the range and any value in the continuum there between. Numerical values provided herein, and the use of the term “about”, may include variations of ±1%, ±2%, ±3%, ±4%, ±5%, and ±10% and their numerical equivalents. “About” when used to modify a numerically defined parameter (e.g., the dose of an antiviral nucleoside, or the length of treatment time with a combination therapy described herein) means that the parameter may vary by as much as 10% below or above the stated numerical value for that parameter; where appropriate, the stated parameter may be rounded to the nearest whole number. For example, a dose of about 5 mg/kg may vary between 4.5 mg/kg and 5.5 mg/kg. In addition, the term “or,” as used herein, denotes alternatives that may, where appropriate, be combined; that is, the term “or” includes each listed alternative separately as well as their combination.

The term “alkyl,” as used herein, refers to an aliphatic hydrocarbon group having one of its hydrogen atoms replaced with a bond having the specified number of carbon atoms. In different embodiments, an alkyl group contains from 1 to 6 carbon atoms (C₁-C₆ alkyl) or from 1 to 3 carbon atoms (C₁-C₃ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl, and neohexyl. In one embodiment, an alkyl group is linear. In another embodiment, an alkyl group is branched.

The terms “halogen” and “halo,” as used herein, means —F (fluorine), —Cl (chlorine), —Br (bromine), or —I (iodine).

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group has from 1 to 3 carbon atoms. In another embodiment, a haloalkyl group is substituted with from 1 to 3 halogen atoms. Non-limiting examples of haloalkyl groups include —CH₂F, —CHF₂, and —CF₃. The term “C₁-C₄ haloalkyl” refers to a haloalkyl group having from 1 to 4 carbon atoms.

The term “alkoxy” as used herein, refers to an —O-alkyl group, wherein an alkyl group is as defined above. Non-limiting examples of alkoxy groups include methoxy, ethoxy, n-propoxy, isopropoxy, n-butoxy, and tert-butoxy. An alkoxy group is bonded via its oxygen atom to the rest of the molecule.

The term “aryl,” as used herein, refers to an aromatic monocyclic or multicyclic ring system comprising from about 6 to about 14 carbon atoms. In one embodiment, an aryl group contains from about 6 to 10 carbon atoms (C₆-C₁₀ aryl). In another embodiment an aryl group is phenyl. Non-limiting examples of aryl groups include phenyl and naphthyl.

When a functional group in a compound is termed “protected,” the group is in modified form to preclude undesired side reactions at the protected site when the compound is subjected to a reaction. The term “PG”, as used herein, refers to a protecting group. Those skilled in the art will readily envisage protecting groups (PG) suitable for use in compounds and processes according to the disclosure. Suitable protecting groups will be recognized by those of ordinary skill in the art as well as by reference to standard textbooks such as, for example, T. W. Greene et al., Protective Groups in Organic Synthesis (1991), Wiley, New York. Protecting groups suitable for use herein include acid-labile protecting groups. Non-limiting examples of PG suitable for use herein include —S(O)₂R⁸, —C(O)OR⁸, —C(O)R⁸, —CH₂OCH₂CH₂SiR⁸, and —CH₂R₈, wherein R⁸ is selected from the group consisting of —C₁₋₈ alkyl (straight or branched), —C₃₋₈ cycloalkyl, —CH₂(aryl), and —CH(aryl)₂, wherein each aryl is independently phenyl or naphthyl and each said aryl is optionally independently unsubstituted or substituted with one or more (e.g., 1, 2, or 3) groups independently is selected from the group consisting of —OCH₃, —Cl, —Br, and —I.

The term “substituted” means that one or more hydrogens on the atoms of the designated moiety are replaced with a selection from the indicated group, provided that the atoms' normal valencies under the existing circumstances are not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

When any substituent or variable occurs more than one time in any compound, its definition on each occurrence is independent of its definition at every other occurrence, unless otherwise indicated. For example, description of radicals that include the expression “—N(C₁-C₃ alkyl)₂” means —N(CH₃)(CH₂CH₃), —N(CH₃)(CH₂CH₂CH₃), and —N(CH₂CH₃)(CH₂CH₂CH₃), as well as —N(CH₃)₂, —N(CH₂CH₃)₂, and —N(CH₂CH₂CH₃)₂.

It should also be noted that any carbon or heteroatom with unsatisfied valences in the text, schemes, examples and Tables herein is assumed to have sufficient hydrogen atom(s) to satisfy the valences. Any one or more of these hydrogen atoms can be deuterium.

The present disclosure also embraces isotopically-labelled compounds that are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²³I, respectively.

Certain isotopically-labelled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Isotopic substitution at a site where epimerization occurs may slow or reduce the epimerization process and thereby retain the more active or efficacious form of the compound for a longer period of time. Isotopically labeled compounds, in particular those containing isotopes with longer half-lives (T_{1/2}>1 day), can generally be prepared by following procedures analogous to those disclosed in the Schemes and/or in the Examples herein below, by substituting an appropriate isotopically labeled reagent for a non-isotopically labeled reagent.

One or more compounds herein may exist in unsolvated as well as solvated forms with pharmaceutically acceptable solvents, such as water, ethanol, and the like, and this disclosure is intended to embrace both solvated and unsolvated forms. “Solvate” means a physical association of a compound with one or more solvent molecules. This physical association involves varying degrees of ionic and covalent bonding, including hydrogen bonding. In certain instances of this aspect, the solvate will be capable of isolation, for example when one or more solvent molecules are incorporated in the crystal lattice of a crystalline solid. “Solvate” encompasses both solution-phase and isolatable solvates. Non-limiting examples of suitable solvates include ethanolates, methanolates, and the like. “Hydrate” is a solvate in which the solvent molecule is H₂O.

Compounds herein may contain one or more stereogenic centers and can thus occur as racemates, racemic mixtures, single enantiomers, diastereomeric mixtures, and individual diastereomers. Additional asymmetric centers may be present depending upon the nature of the various substituents on the molecule. Each such asymmetric center will independently produce two optical isomers, and all possible optical isomers and diastereomers in mixtures and as pure or partially purified compounds are included within the disclosure. Any formulas, structures, or names of compounds described herein that do not specify a particular stereochemistry are meant to encompass any and all existing isomers as described above and mixtures thereof in any proportion. When stereochemistry is specified, the disclosure is meant to encompass that particular isomer in pure form or as part of a mixture with other isomers in any proportion.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Enantiomers can also be separated by use of chiral HPLC column.

All stereoisomers (for example, geometric isomers, optical isomers, and the like) of disclosed compounds (including those of the salts and solvates of compounds as well as the salts, solvates, and esters of prodrugs), such as those that may exist due to asymmetric carbons on various substituents, including enantiomeric forms (which may exist even in the absence of asymmetric carbons), rotameric forms, atropisomers, and diastereomeric forms, are contemplated within the scope of this disclosure. Individual stereoisomers of compounds may, for example, be substantially free of other isomers, or may be admixed, for example, as racemates or with all other, or other selected, stereoisomers. The chiral centers can have the S or R configuration as defined by the IUPAC 1974 Recommendations.

The present disclosure further includes compounds and synthetic intermediates in all their isolated forms. For example, the identified compounds are intended to encompass all forms of the compounds such as, any solvates, hydrates, stereoisomers, and tautomers thereof.

Those skilled in the art will recognize that certain compounds, and in particular compounds containing certain heteroatoms and double or triple bonds, can be tautomers, structural isomers that readily interconvert. Thus, tautomeric compounds can be drawn in a number of different ways that are equivalent. Non-limiting examples of such tautomers include those exemplified below.

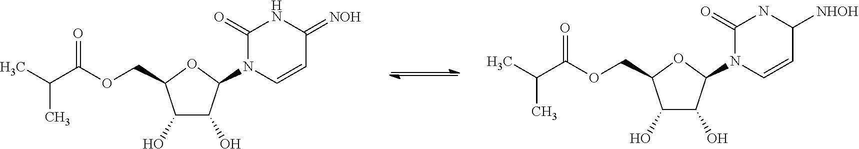

Those skilled in the art will recognize that chiral compounds, and in particular sugars, can be drawn in a number of different ways that are equivalent. Those skilled in the art will further recognize that the identity and regiochemical position of the substituents on ribose can vary widely and that the same principles of stereochemical equivalence apply regardless of substituent. Non-limiting examples of such equivalence include those exemplified below.

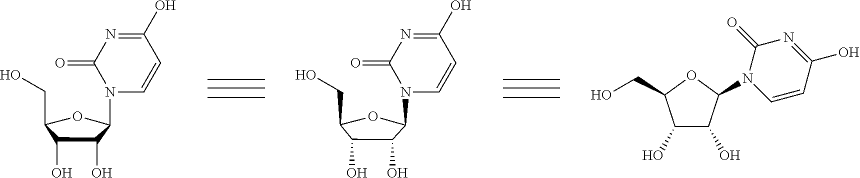

Compounds can form salts that are also within the scope of this disclosure. Reference to a compound herein is understood to include reference to salts thereof, unless otherwise indicated. The term “salt(s),” as employed herein, denotes acidic salts formed with inorganic and/or organic acids, as well as basic salts formed with inorganic and/or organic bases. In addition, when a compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds may be formed, for example, by reacting a compound with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates), and the like. Additionally, acids that are generally considered suitable for the formation of pharmaceutically useful salts from basic pharmaceutical compounds are discussed, for example, by P. Stahl et al., Camille G. (eds.) Handbook of Pharmaceutical Salts: Properties, Selection and Use (2002) Zurich: Wiley-VCH; S. Berge et al., J. Pharm. Sci. (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C.). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, tert-butyl amines, and salts with amino acids such as arginine, lysine, and the like. Basic nitrogen-containing groups may be quarternized with agents such as lower alkyl halides (e.g. methyl, ethyl, and butyl chlorides, bromides, and iodides), dialkyl sulfates (e.g. dimethyl, diethyl, and dibutyl sulfates), long chain halides (e.g. decyl, lauryl, and stearyl chlorides, bromides, and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

“Protein,” “polypeptide,” and “peptide” are used interchangeably herein to denote a polymer of at least two amino acids covalently linked by an amide bond, regardless of length or post-translational modification (e.g., glycosylation or phosphorylation, lipidation, myristoylation, ubiquitination, etc.). Included within this definition are D- and L-amino acids, and mixtures of D- and L-amino acids, as well as polymers comprising D- and L-amino acids, and mixtures of D- and L-amino acids. Proteins, polypeptides, and peptides may include a tag, such as a histidine tag, which should not be included when determining percentage of sequence identity.

“Amino acid” or “residue” as used in context of the polypeptides disclosed herein refers to the specific monomer at a sequence position. Amino acids are referred to herein by either their commonly known three-letter symbols or by the one-letter symbols recommended by IUPAC-IUB Biochemical Nomenclature Commission. Nucleotides, likewise, may be referred to by their commonly accepted single letter codes.

The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V).

The abbreviations used for the genetically encoding nucleosides are conventional and are as follows: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When nucleic acid sequences are presented as a string of one-letter abbreviations, the sequences are presented in the 5′ to 3′ direction in accordance with common convention, and the phosphates are not indicated.

“Derived from” as used herein in the context of enzymes, identifies the originating enzyme, and/or the gene encoding such enzyme, upon which the enzyme was based. For example, the MTR kinase of SEQ ID NO: 7 was obtained by artificially evolving, over multiple generations the gene encoding the MTR kinase enzyme of SEQ ID NO: 1. Thus, this evolved MTR kinase enzyme is “derived from” the MTR kinase of SEQ ID NO: 1.

“Hydrophilic amino acid or residue” refers to an amino acid or residue having a side chain exhibiting a hydrophobicity of less than zero according to the normalized consensus hydrophobicity scale of Eisenberg et al., 1984, J. MOL. BIOL. 179:125-142. Genetically encoded hydrophilic amino acids include L-Thr (T), L-Ser (S), L-His (H), L-Glu (E), L-Asn (N), L-Gln (Q), L-Asp (D), L-Lys (K), and L-Arg (R).

“Acidic amino acid or residue” refers to a hydrophilic amino acid or residue having a side chain exhibiting a pK value of less than about 6 when the amino acid is included in a peptide or polypeptide. Acidic amino acids typically have negatively charged side chains at physiological pH due to loss of a hydrogen ion. Genetically encoded acidic amino acids include L-Glu (E) and L-Asp (D).

“Basic amino acid or residue” refers to a hydrophilic amino acid or residue having a side chain exhibiting a pKa value of greater than about 6 when the amino acid is included in a peptide or polypeptide. Basic amino acids typically have positively charged side chains at physiological pH due to association with hydronium ion. Genetically encoded basic amino acids include L-Arg (R) and L-Lys (K).

“Polar amino acid or residue” refers to a hydrophilic amino acid or residue having a side chain that is uncharged at physiological pH, but which has at least one bond in which the pair of electrons shared in common by two atoms is held more closely by one of the atoms. Genetically encoded polar amino acids include L-Asn (N), L-Gln (Q), L-Ser (S), and L-Thr (T).

“Hydrophobic amino acid or residue” refers to an amino acid or residue having a side chain exhibiting a hydrophobicity of greater than zero according to the normalized consensus hydrophobicity scale of Eisenberg et al., 1984, J. MOL. BIOL. 179:125-142. Genetically encoded hydrophobic amino acids include L-Pro (P), L-Ile (I), L-Phe (F), L-Val (V), L-Leu (L), L-Trp (W), L-Met (M), L-Ala (A), and L-Tyr (Y).

“Aromatic amino acid or residue” refers to a hydrophilic or hydrophobic amino acid or residue having a side chain that includes at least one aromatic or heteroaromatic ring. Genetically encoded aromatic amino acids include L-Phe (F), L-Tyr (Y), L-His (H), and L-Trp (W). L-His (H) histidine is also classified herein as a hydrophilic residue or as a constrained residue.

As used herein, “constrained amino acid or residue” refers to an amino acid or residue that has a constrained geometry. Herein, constrained residues include L-Pro (P) and L-His (H). Histidine has a constrained geometry because it has a relatively small imidazole ring. Proline has a constrained geometry because it also has a five-membered ring.

“Non-polar amino acid or residue” refers to a hydrophobic amino acid or residue that has a side chain that is uncharged at physiological pH and that has bonds in which the pair of electrons shared in common by two atoms is generally held equally by each of the two atoms (i.e., the side chain is not polar). Genetically encoded non-polar amino acids include L-Gly (G), L-Leu (L), L-Val (V), L-Ile (I), L-Met (M), and L-Ala (A).

As used herein, “aliphatic amino acid or residue” refers to a hydrophobic amino acid or residue having an aliphatic hydrocarbon side chain. Genetically encoded aliphatic amino acids include L-Ala (A), L-Val (V), L-Leu (L), and L-Ile (I).

The ability of L-Cys (C) (and other amino acids with —SH containing side chains) to exist in a peptide in either the reduced free —SH or oxidized disulfide-bridged form affects whether L-Cys (C) contributes net hydrophobic or hydrophilic character to a peptide. While L-Cys (C) exhibits a hydrophobicity of 0.29 according to the normalized consensus scale of Eisenberg (Eisenberg et al., 1984, supra), it is to be understood that for purposes of the present disclosure, L-Cys (C) is categorized into its own unique group. It is noted that cysteine (or “L-Cys” or “[C]”) is unusual in that it can form disulfide bridges with other L-Cys (C) amino acids or other sulfanyl- or sulfhydryl-containing amino acids. The “cysteine-like residues” include cysteine and other amino acids that contain sulfhydryl moieties that are available for formation of disulfide bridges.

As used herein, “small amino acid or residue” refers to an amino acid or residue having a side chain that is composed of a total three or fewer carbon and/or heteroatoms (excluding the α-carbon and hydrogens). The small amino acids or residues may be further categorized as aliphatic, non-polar, polar or acidic small amino acids or residues, in accordance with the above definitions. Genetically-encoded small amino acids include L-Ala (A), L-Val (V), L-Cys (C), L-Asn (N), L-Ser (S), L-Thr (T), and L-Asp (D).

“Hydroxyl-containing amino acid or residue” refers to an amino acid containing a hydroxyl (—OH) moiety. Genetically-encoded hydroxyl-containing amino acids include L-Ser (S) L-Thr (T), and L-Tyr (Y).

As used herein, “polynucleotide” and “nucleic acid” refer to two or more nucleotides that are covalently linked together. The polynucleotide may be wholly comprised of ribonucleotides (i.e., RNA), wholly comprised of 2′ deoxyribonucleotides (i.e., DNA), or comprised of mixtures of ribo- and 2′ deoxyribonucleotides. While the nucleosides will typically be linked together via standard phosphodiester linkages, the polynucleotides may include one or more non-standard linkages. The polynucleotide may be single-stranded or double-stranded, or the polynucleotide may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. In some embodiments, such modified or synthetic nucleobases are nucleobases encoding amino acid sequences.

As used herein, “nucleoside” refers to glycosylamines comprising a nucleobase (i.e., a nitrogenous base), and a 5-carbon sugar (e.g., ribose or deoxyribose). Non-limiting examples of nucleosides include cytidine, uridine, adenosine, guanosine, thymidine, and inosine. In contrast, the term “nucleotide” refers to the glycosylamines comprising a nucleobase, a 5-carbon sugar, and one or more phosphate groups. In some embodiments, nucleosides can be phosphorylated by kinases to produce nucleotides.

As used herein, “nucleoside diphosphate” refers to glycosylamines comprising a nucleobase (i.e., a nitrogenous base), a 5-carbon sugar (e.g., ribose or deoxyribose), and a diphosphate (i.e., pyrophosphate) moiety. In some embodiments herein, “nucleoside diphosphate” is abbreviated as “NDP.” Non-limiting examples of nucleoside diphosphates include cytidine diphosphate (CDP), uridine diphosphate (UDP), adenosine diphosphate (ADP), guanosine diphosphate (GDP), thymidine diphosphate (TDP), and inosine diphosphate (IDP). The terms “nucleoside” and “nucleotide” may be used interchangeably in some contexts.

As used herein, “nucleoside triphosphate” refers to glycosylamines comprising a nucleobase (i.e., a nitrogenous base), a 5-carbon sugar (e.g., ribose or deoxyribose), and a triphosphate moiety. In some embodiments herein, “nucleoside triphosphate” is abbreviated as “NTP.” Non-limiting examples of nucleoside triphosphates include cytidine triphosphate (CTP), uridine triphosphate (UTP), adenosine triphosphate (ATP), guanosine triphosphate (GTP), thymidine triphosphate (TTP), and inosine triphosphate (ITP). The terms “nucleoside” and “nucleotide” may be used interchangeably in some contexts.

As used herein, “conservative amino acid substitution” refers to a substitution of a residue with a different residue having a similar side chain, and thus typically involves substitution of the amino acid in the polypeptide with amino acids within the same or similar defined class of amino acids. By way of example and not limitation, in some embodiments, an amino acid with an aliphatic side chain is substituted with another aliphatic amino acid (e.g., alanine, valine, leucine, and isoleucine); an amino acid with an hydroxyl side chain is substituted with another amino acid with an hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basic side chain (e.g., lysine and arginine); an amino acid with an acidic side chain is substituted with another amino acid with an acidic side chain (e.g., aspartic acid and glutamic acid); and/or a hydrophobic or hydrophilic amino acid is replaced with another hydrophobic or hydrophilic amino acid, respectively.

As used herein, “non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

As used herein, “deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an evolved enzyme. Deletions can be directed to the internal portions and/or terminal portions of the polypeptide. In various embodiments, the deletion can comprise a continuous segment or can be discontinuous. Deletions are typically indicated by “-” in amino acid sequences.

As used herein, “insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

The term “amino acid substitution set” or “substitution set” refers to a group of amino acid substitutions in a polypeptide sequence, as compared to a reference sequence. A substitution set can have 1, 2, 3, 4, 5, 6, 7, 8, 9, 10, 11, 12, 13, 14, 15, or more amino acid substitutions.

A “functional fragment” and “biologically active fragment” are used interchangeably herein to refer to a polypeptide that has an amino-terminal and/or carboxy-terminal deletion(s) and/or internal deletions, but where the remaining amino acid sequence is identical to the corresponding positions in the sequence to which it is being compared and that retains substantially all of the activity of the full-length polypeptide.

As used herein, “isolated polypeptide” refers to a polypeptide that is substantially separated from other contaminants that naturally accompany it (e.g., protein, lipids, and polynucleotides). The term embraces polypeptides that have been removed or purified from their naturally occurring environment or expression system (e.g., within a host cell or via in vitro synthesis). The recombinant polypeptides may be present within a cell, present in the cellular medium, or prepared in various forms, such as lysates or isolated preparations. As such, in some embodiments, the recombinant polypeptides can be an isolated polypeptide.

As used herein, “substantially pure polypeptide” or “purified protein” refers to a composition in which the polypeptide species is the predominant species present (i.e., on a molar or weight basis it is more abundant than any other individual macromolecular species in the composition) and is generally a substantially purified composition when the object species comprises at least about 50 percent of the macromolecular species present by mole or % weight. However, in some embodiments, an enzyme comprising composition comprises enzymes that are less than 50% pure (e.g., about 10%, about 20%, about 30%, about 40%, or about 50%). Generally, a substantially pure enzyme or polypeptide composition comprises about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant polypeptides are substantially pure polypeptide compositions.

“Improved enzyme property” refers to an enzyme that exhibits an improvement in any enzyme property as compared to a reference enzyme. For the enzymes described herein, the comparison is generally made to the wild-type enzyme, although in some embodiments, the reference enzyme can be another improved enzyme. Enzyme properties for which improvement is desirable include, but are not limited to, enzymatic activity (which can be expressed in terms of percent conversion of the substrate), thermal stability, pH activity profile, cofactor requirements, refractoriness to inhibitors (e.g., product inhibition), stereospecificity, and stereoselectivity (including enantioselectivity).

“Increased enzymatic activity” refers to an improved property of the enzymes, which can be represented by an increase in specific activity (e.g., product produced/time/weight protein) or an increase in percent conversion of the substrate to the product (e.g., percent conversion of starting amount of substrate to product in a specified time period using a specified amount of enzyme) as compared to the reference enzyme. Exemplary methods to determine enzyme activity are provided in the Examples. Any property relating to enzyme activity may be affected, including the classical enzyme properties of Kₘ, Vₘₐₓ, or k_{cat}, changes of which can lead to increased enzymatic activity. Improvements in enzyme activity can be from about 1.5 times the enzymatic activity of the corresponding wild-type enzyme, to as much as 2 times. 5 times, 10 times, 20 times, 25 times, 50 times, 75 times, 100 times, 150 times, 200 times, 500 times, 1000 times, 3000 times, 5000 times, 7000 times or more enzymatic activity than the naturally occurring enzyme or another enzyme from which the polypeptides were derived. In specific embodiments, the enzyme exhibits improved enzymatic activity in the range of 150 to 3000 times, 3000 to 7000 times, or more than 7000 times greater than that of the parent enzyme. It is understood by the skilled artisan that the activity of any enzyme is diffusion limited such that the catalytic turnover rate cannot exceed the diffusion rate of the substrate, including any required cofactors. The theoretical maximum of the diffusion limit, or k_{cat}/Kₘ, is generally about 10⁸ to 10⁹ (M⁻¹s⁻¹). Hence, any improvements in the enzyme activity will have an upper limit related to the diffusion rate of the substrates acted on by the enzyme. Enzyme activity can be measured by any one of standard assays used for measuring kinase activity, or via a coupled assay with a nucleoside phosphorylase enzyme which is capable of catalyzing reaction between the polypeptide product and a nucleoside base to afford a nucleoside, or by any of the traditional methods for assaying chemical reactions, including but not limited to HPLC, HPLC-MS, UPLC, UPLC-MS, TLC, and NMR. Comparisons of enzyme activities are made using a defined preparation of enzyme, a defined assay under a set condition, and one or more defined substrates, as further described in detail herein. Generally, when lysates are compared, the numbers of cells and the amount of protein assayed are determined as well as use of identical expression systems and identical host cells to minimize variations in amount of enzyme produced by the host cells and present in the lysates.

As used herein, a “vector” is a DNA construct for introducing a DNA sequence into a cell. In some embodiments, the vector is an expression vector that is operably linked to a suitable control sequence capable of effecting the expression in a suitable host of the polypeptide encoded in the DNA sequence. In some embodiments, an “expression vector” has a promoter sequence operably linked to the DNA sequence (e.g., transgene) to drive expression in a host cell, and in some embodiments, also comprises a transcription terminator sequence.

As used herein, the term “expression” includes any step involved in the production of the polypeptide including, but not limited to, transcription, post-transcriptional modification, translation, and post-translational modification. In some embodiments, the term also encompasses secretion of the polypeptide from a cell.

As used herein, the term “produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

As used herein, an amino acid or nucleotide sequence (e.g., a promoter sequence, signal peptide, terminator sequence, etc.) is “heterologous” to another sequence with which it is operably linked if the two sequences are not associated in nature. For example, a “heterologous polynucleotide” is any polynucleotide that is introduced into a host cell by laboratory techniques, and the term includes polynucleotides that are removed from a host cell, subjected to laboratory manipulation, and then reintroduced into a host cell.

As used herein, the terms “host cell” and “host strain” refer to suitable hosts for expression vectors comprising DNA provided herein (e.g., the polynucleotides encoding the variants). In some embodiments, the host cells are prokaryotic or eukaryotic cells that have been transformed or transfected with vectors constructed using recombinant DNA techniques as known in the art.

The term “analogue” means a polypeptide having more than 70% sequence identity but less than 100% sequence identity (e.g., more than 75%, 78%, 80%, 83%, 85%, 88%, 90%, 91%, 92%, 93%, 94%, 95%, 96%, 97%, 98%, or 99% sequence identity) with a reference polypeptide. In some embodiments, “analogues” means polypeptides that contain one or more non-naturally occurring amino acid residues including, but not limited, to homoarginine, ornithine and norvaline, as well as naturally occurring amino acids. In some embodiments, analogues also include one or more D-amino acid residues and non-peptide linkages between two or more amino acid residues.

As used herein, “EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

As used herein, “ATCC” refers to the American Type Culture Collection whose biorepository collection includes genes and strains.

As used herein, “NCBI” refers to National Center for Biological Information and the sequence databases provided therein.

“Coding sequence” refers to that portion of a nucleic acid (e.g., a gene) that encodes an amino acid sequence of a protein.

“Naturally occurring” or “wild-type” refers to a form found in nature. For example, a naturally occurring or wild-type polypeptide or polynucleotide sequence is a sequence present in an organism that can be isolated from a source in nature and that has not been intentionally modified by human manipulation, with the sole exception that wild-type polypeptide or polynucleotide sequences as identified herein may include a tag, such as a histidine tag, which should not be included when determining percentage of sequence identity. Herein, “wild-type” polypeptide or polynucleotide sequences may be denoted “WT”.

“Recombinant” when used with reference to, e.g., a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Percentage of sequence identity,” “percent identity,” and “percent identical” are used herein to refer to comparisons between polynucleotide sequences or polypeptide sequences and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (see e.g., Altschul et al., 1990, J. MOL. BIOL. 215: 403-410; and Altschul et al., 1977, NUCLEIC ACIDS RES. 3389-3402). Software for performing BLAST analyses is publicly available through the National Center for Biotechnology Information website.

Briefly, the BLAST analyses involve first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff and Henikoff, 1989, PROC. NATL. ACAD. SCI. USA 89:10915).

Numerous other algorithms are available that function similarly to BLAST in providing percent identity for two sequences. Optimal alignment of sequences for comparison can be conducted, e.g., by the local homology algorithm of Smith and Waterman, 1981, ADV. APPL. MATH. 2:482, by the homology alignment algorithm of Needleman and Wunsch, 1970, J. MOL. BIOL. 48:443, by the search for similarity method of Pearson and Lipman, 1988, N USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Additionally, determination of sequence alignment and percent sequence identity can employ the BESTFIT or GAP programs in the GCG Wisconsin Software package (Accelrys, Madison WI), using default parameters provided.

“Substantial identity” refers to a polynucleotide or polypeptide sequence that has at least 80 percent sequence identity, preferably at least 85 percent sequence identity, more preferably at least 89 percent sequence identity, more preferably at least 95 percent sequence identity, and even more preferably at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In specific embodiments applied to polypeptides, the term “substantial identity” means that two polypeptide sequences, when optimally aligned, such as by the programs GAP or BESTFIT using default gap weights, share at least 80 percent sequence identity, preferably at least 89 percent sequence identity, more preferably at least 95 percent sequence identity or more (e.g., 99 percent sequence identity). Preferably, residue positions which are not identical differ by conservative amino acid substitutions.

“Corresponding to”, “reference to”, or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Stereoselectivity” refers to the preferential formation in a chemical or enzymatic reaction of one stereoisomer over another. Stereoselectivity can be partial, where the formation of one stereoisomer is favored over the other, or it may be complete where only one stereoisomer is formed. When the stereoisomers are enantiomers, the stereoselectivity is referred to as enantioselectivity, the fraction (typically reported as a percentage) of one enantiomer in the sum of both. It is commonly alternatively reported in the art (typically as a percentage) as the enantiomeric excess (EE) calculated therefrom according to the formula [major enantiomer−minor enantiomer]/[major enantiomer+minor enantiomer]. Where the stereoisomers are diastereoisomers, the stereoselectivity is referred to as diastereoselectivity, the fraction (typically reported as a percentage) of one diastereomer in a mixture of two diastereomers, commonly alternatively reported as the diastereomeric excess (DE). Enantiomeric excess and diastereomeric excess are types of stereometric excess.

“Highly stereoselective” refers to a chemical or enzymatic reaction that is capable of converting a substrate to its corresponding product with at least about 85% stereoisomeric excess.

“Chemoselectivity” refers to the preferential formation in a chemical or enzymatic reaction of one product over another.

“Conversion” refers to the enzymatic transformation of a substrate to the corresponding product. “Percent conversion” refers to the percent of the substrate that is converted to the product within a period of time under specified conditions. Thus, for example, the “enzymatic activity” or “activity” of a polypeptide can be expressed as “percent conversion” of the substrate to the product.

“Chiral alcohol” refers to amines of general formula R¹—CH(OH)—R² wherein R¹ and R² are nonidentical and is employed herein in its broadest sense, including a wide variety of aliphatic and alicyclic compounds of different, and mixed, functional types, characterized by the presence of a primary hydroxyl group bound to a secondary carbon atom which, in addition to a hydrogen atom, carries either (i) a divalent group forming a chiral cyclic structure, or (ii) two substituents (other than hydrogen) differing from each other in structure or chirality. Divalent groups forming a chiral cyclic structure include, for example, 2-methylbutane-1,4-diyl, pentane-1,4-diyl, hexane-1,4-diyl, hexane-1,5-diyl, 2-methylpentane-1,5-diyl. The two different substituents on the secondary carbon atom (R¹ and R² above) also can vary widely and include alkyl, aralkyl, aryl, halo, hydroxy, lower alkyl, lower alkoxy, lower alkylthio, cycloalkyl, carboxy, carboalkoxy, carbamoyl, mono- and di-(lower alkyl) substituted carbamoyl, trifluoromethyl, phenyl, nitro, amino, mono- and di-(lower alkyl) substituted amino, alkylsulfonyl, arylsulfonyl, alkylcarboxamido, arylcarboxamido, etc., as well as alkyl, aralkyl, or aryl substituted by the foregoing.

Immobilized enzyme preparations have a number of recognized advantages. They can confer shelf life to enzyme preparations, they can improve reaction stability, they can enable stability in organic solvents, they can aid in protein removal from reaction streams, as examples. “Stable” refers to the ability of the immobilized enzymes to retain their structural conformation and/or their activity in a solvent system that contains organic solvents. Stable immobilized enzymes lose less than 10% activity per hour in a solvent system that contains organic solvents. Stable immobilized enzymes lose less than 9% activity per hour in a solvent system that contains organic solvents. Preferably, the stable immobilized enzymes lose less than 8% activity per hour in a solvent system that contains organic solvents. Preferably, the stable immobilized enzymes lose less than 7% activity per hour in a solvent system that contains organic solvents. Preferably, the stable immobilized enzymes lose less than 6% activity per hour in a solvent system that contains organic solvents. Preferably, the stable immobilized enzymes lose less than 5% activity per hour in a solvent system that contains organic solvents. Preferably, the stable immobilized enzymes less than 4% activity per hour in a solvent system that contains organic solvents. Preferably, the stable immobilized enzymes lose less than 3% activity per hour in a solvent system that contains organic solvents. Preferably, the stable immobilized enzymes lose less than 2% activity per hour in a solvent system that contains organic solvents. Preferably, the stable immobilized enzymes lose less than 1% activity per hour in a solvent system that contains organic solvents.

“Thermostable” refers to a polypeptide that maintains similar activity (more than 60% to 80% for example) after exposure to elevated temperatures (e.g., 40° to 80° C.) for a period of time (e.g., 0.5 h to 24 h) compared to the untreated enzyme.

“Solvent stable” refers to a polypeptide that maintains similar activity (more than e.g., 60% to 80%) after exposure to varying concentrations (e.g., 5% to 99%) of solvent (isopropyl alcohol, tetrahydrofuran, 2-methyltetrahydrofuran, acetone, toluene, butylacetate, methyl tert-butylether, etc.) for a period of time (e.g., 0.5 h to 24 h) compared to the untreated enzyme.

“pH stable” refers to a polypeptide that maintains similar activity (more than e.g., 60% to 80%) after exposure to high or low pH (e.g., 4.5 to 6 or 8 to 12) for a period of time (e.g., 0.5 h to 24 h) compared to the untreated enzyme.

“Thermo- and solvent stable” refers to a polypeptide that is both thermostable and solvent stable.

As used herein, the terms “biocatalysis,” “biocatalytic,” “biotransformation,” and “biosynthesis” refer to the use of enzymes to perform chemical reactions on organic compounds.

The term “effective amount” means an amount sufficient to produce the desired result. One of general skill in the art may determine what the effective amount by using routine experimentation.

The terms “isolated” and “purified” are used to refer to a molecule (e.g., an isolated nucleic acid, polypeptide, etc.) or other component that is removed from at least one other component with which it is naturally associated. The term “purified” does not require absolute purity, rather it is intended as a relative definition.

Exemplary methods and materials are described herein, although methods and materials similar or equivalent to those described herein can also be used in the practice or testing of the present disclosure. The materials, methods, and examples are illustrative only and not intended to be limiting.

### Antiviral Nucleosides

As used herein, “antiviral nucleosides” means any nucleoside chemical compound that exhibits antiviral activity, and in particular, the antiviral nucleosides as disclosed in PCT International Patent Application No. PCT/US2018/064503, which published as PCT International Patent Application Publication No. WO2019/113462, which is incorporated herein by reference in its entirety. In particular, the term “antiviral nucleosides” includes Compound B and pharmaceutically acceptable salts, derivatives, tautomers, isomers, and prodrugs of Compound B, as well as particular forms as disclosed in PCT International Patent Application No. PCT/US2021/048054, filed Aug. 27, 2021. In addition, the term “antiviral nucleosides” includes pharmaceutically acceptable salts, derivatives, or prodrugs of such compounds. Antiviral nucleosides, and particularly Compound B and pharmaceutically acceptable salts, derivatives, tautomers, isomers, and prodrugs of Compound B, may be used in therapeutic combinations for antiviral treatment.

### Processes for Preparing Antiviral Nucleosides

The present disclosure provides processes for preparing Compound B and pharmaceutically acceptable salts, derivatives, tautomers, isomers, and prodrugs thereof:

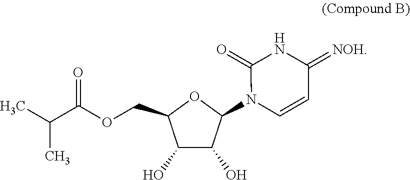

In embodiments, the processes of the disclosure may be conducted in a single vessel, as a “one-pot” process, or the steps may be conducted sequentially. In embodiments, the intermediate products may optionally be isolated.

A first embodiment of the processes of the disclosure comprises reacting 5′-isobutyryluridine with at least one hydroxylamine source in the presence of at least one activating agent to produce Compound B:

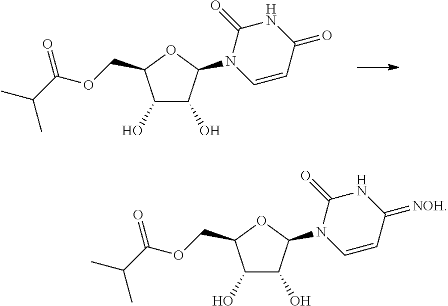

In a first aspect of this first embodiment, the at least one hydroxylamine source is selected from the group consisting of hydroxylamine sulfate, hydroxylamine HCl, NH₂OH, and mixtures thereof. In instances of this first aspect, the at least one hydroxylamine source is hydroxylamine sulfate. In specific instances of this aspect, the at least one hydroxylamine source is provided in an amount to provide hydroxylamine in a range of from about 1.0 to about 2.5 equivalents of hydroxylamine with respect to the amount of 5′-isobutyryluridine, such as an amount in a range of from about 2.2 to about 2.5 equivalents, or an amount of about 2.5 equivalents.

In a second aspect of this first embodiment, the at least one activating agent is hexamethyldisilazane. In instances of this aspect, the at least one activating agent is provided in an amount in a range of from about 6.0 to about 8.0 equivalents with respect to the amount of 5′-isobutyryluridine, such as an amount of about 8 equivalents.

In a third aspect of this first embodiment, the reacting is conducted in the presence of at least one acidic additive. In instances of this third aspect, the at least one acidic additive is selected from the group consisting of ammonium bisulfate, ammonium dihydrogen phosphate, sulfuric acid, sodium bisulfate, potassium bisulfate, imidazole bisulfate, triethylamine bisulfate, N-methylmorpholine bisulfate, N-methylimidazole bisulfate, sulfur trioxide pyridine complex, trifluoromethanesulfonic acid, methanesulfonic acid, and mixtures thereof. In specific instances, the at least one acidic additive is ammonium bisulfate. In specific instances, the at least one acidic additive is present in an amount in a range of from about 1.5 to about 3.0 equivalents with respect to the amount of 5′-isobutyryluridine.

In a fourth aspect of this first embodiment, the reacting is optionally conducted in the presence of at least one catalyst. In instances of this aspect, the at least one catalyst is selected from Lewis basic catalysts. In occurrences of these instances, the at least one catalyst is selected from the group consisting of N-methylimidazole, N-methylmorpholine, 1,2,4-triazole, 5-(ethylthio)-1H-tetrazole, imidazole, and mixtures thereof. In specific occurrences, the at least one catalyst is imidazole. In specific occurrences, the at least one catalyst is present in an amount in a range of from about 0.2 to about 0.5 equivalents with respect to the amount of 5′-isobutyryluridine.

In a fifth aspect of this first embodiment, the reacting is conducted in the presence of at least one solvent. In instances of this aspect, the at least one solvent is selected from water, organic solvents and mixtures thereof. In particular instances of this aspect, the at least one solvent is at least one organic solvent selected from the group consisting of heptane, toluene, 2-methyltetrahydrofuran, tetrahydrofuran, DME, sulfolane, and mixtures thereof. In other specific instances, the at least one solvent is selected from mixtures of water and organic solvents. In particular occurrences of such instances, the at least one solvent is selected from mixtures of water and at least one organic solvent selected from the group consisting of heptane, toluene, 2-methyltetrahydrofuran, tetrahydrofuran, DME, sulfolane, and mixtures thereof.

A second embodiment of the processes of the disclosure, the process of the first embodiment comprises reacting 5-isobutyrylribose with uracil in the presence of at least one enzyme to form 5′-isobutyryluridine:

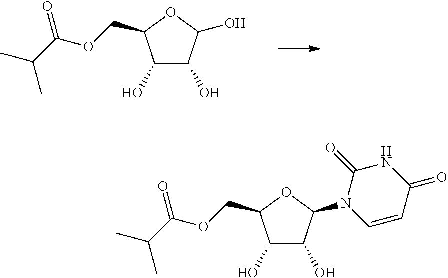 [SMILES: CC(C)C(=O)OC[C@H]1OC(O)[C@H](O)[C@@H]1O ; CC(C)C(=O)OC[C@H]1O[C@@H](n2ccc(=O)[nH]c2=O)[C@H](O)[C@@H]1O]

In a first aspect of this second embodiment, uracil is provided in an amount in a range of from about 0.5 to about 1.2 equivalents with respect to the amount of 5′-isobutyrylribose, such as an amount of about 0.8 equivalents.

In a second aspect of this second embodiment, the at least one enzyme (alternatively “an enzyme” or “enzymes”) is selected from the group consisting of at least one S-methyl-5-thioribose kinase enzyme (alternatively referred to as “S-methyl-5-thioribose kinase enzymes”), at least one acetate kinase enzyme (alternatively referred to as “acetate kinase enzymes”), at least one pyruvate oxidase enzyme (alternatively referred to as “pyruvate oxidase enzymes”), at least one catalase enzyme (alternatively referred to as “catalase enzymes”), at least one uridine phosphorylase enzyme (alternatively referred to as “uridine phosphorylase enzymes”), and mixtures thereof. In instances of this second aspect, the at least one enzyme is selected from the group consisting of S-methyl-5-thioribose kinase enzymes, acetate kinase enzymes, pyruvate oxidase enzymes, catalase enzymes, and uridine phosphorylase enzymes, and mixtures thereof.

In a first instance of the second aspect of this second embodiment, the at least one S-methyl-5-thioribose kinase enzyme is one or more S-methyl-5-thioribose kinase selected from the group consisting of wild-type S-methyl-5-thioribose kinase enzymes and S-methyl-5-thioribose kinase enzymes that are produced from the directed evolution from a commercially available, wild-type S-methyl-5-thioribose kinase enzymes. In specific instances, the at least one S-methyl-5-thioribose kinase enzyme is selected from the group consisting of S-methyl-5-thioribose kinase enzymes that are produced from the directed evolution from a commercially available, wild-type S-methyl-5-thioribose kinase enzyme, which has the amino acid sequence as set forth below in SEQ ID NO: 1.

(SEQ ID NO: 1)
MSQYHTFTAHDAVAYAQQFAGIDNPSELVSAQEVGDGNLNLVFKVFDRQG
VSRAIVKQALPYVRCVGESWPLTLDRARLEAQTLVAHYQHSPQHTVKIHH
FDPELAVMVMEDLSDHRIWRGELIANVYYPQAARQLGDYLAQVLFHTSDF
YLHPHEKKAQVAQFINPAMCEITEDLFFNDPYQIHERNNYPAELEADVAA
LRDDAQLKLAVAALKHRFFAHAEALLHGDIHSGSIFVAEGSLKAIDAEFG
YFGPIGFDIGTAIGNLLLNYCGLPGQLGIRDAAAAREQRLNDIHQLWTTF
AERFQALAAEKTRDAALAYPGYASAFLKKVWADAVGFCGSELIRRSVGLS
HVADIDTIQDDAMRHECLRHAITLGRALIVLAERIDSVDELLARVRQYS

In occurrences, the at least one S-methyl-5-thioribose kinase enzyme is the wild-type S-methyl-5-thioribose kinase having the amino acid sequence as set forth above in SEQ ID NO: 1. In specific occurrences, the wild-type S-methyl-5-thioribose kinase may be encoded by the DNA sequence as set forth below in SEQ ID NO: 2.

(SEQ ID NO: 2)
ATGAGCCAGTATCATACCTTCACCGCGCATGATGCGGTGGCGTATGCGCA
GCAATTTGCGGGCATTGATAACCCGAGCGAGCTGGTTAGCGCGCAAGAAG
TTGGTGACGGCAACCTGAACCTGGTGTTCAAGGTTTTTGATCGTCAGGGT
GTGAGCCGTGCGATCGTTAAACAAGCGCTGCCGTACGTGCGTTGCGTTGG
TGAAAGCTGGCCGCTGACCCTGGACCGTGCGCGTCTGGAAGCGCAGACCC
TGGTGGCGCACTATCAGCACAGCCCGCAACACACCGTTAAGATCCACCAC
TTCGATCCGGAGCTGGCGGTGATGGTTATGGAAGACCTGAGCGATCACCG
TATTTGGCGTGGTGAGCTGATCGCGAACGTGTACTATCCGCAGGCGGCGC
GTCAACTGGGTGACTACCTGGCGCAGGTTCTGTTCCACACCAGCGATTTT
TATCTGCACCCGCACGAGAAGAAAGCGCAGGTGGCGCAATTCATTAACCC
GGCGATGTGCGAGATCACCGAAGACCTGTTCTTTAACGATCCGTACCAGA
TTCACGAACGTAACAACTATCCGGCGGAGCTGGAAGCGGATGTGGCGGCG
CTGCGTGATGATGCGCAACTGAAGCTGGCGGTTGCGGCGCTGAAACACCG
TTTCTTTGCGCATGCGGAGGCGCTGCTGCATGGTGACATTCACAGCGGCA
GCATCTTCGTTGCGGAGGGTAGCCTGAAGGCGATCGACGCGGAATTCGGT
TACTTTGGCCCGATCGGTTTTGATATTGGTACCGCGATCGGCAACCTGCT
GCTGAACTATTGCGGTCTGCCGGGTCAACTGGGTATTCGTGATGCGGCGG
CGGCGCGTGAACAGCGTCTGAACGATATCCACCAACTGTGGACCACCTTC
GCGGAGCGTTTTCAAGCGCTGGCGGCGGAAAAGACCCGTGACGCGGCGCT
GGCGTACCCGGGTTATGCGAGCGCGTTCCTGAAGAAAGTGTGGGCGGATG
CGGTTGGTTTTTGCGGCAGCGAGCTGATTCGTCGTAGCGTGGGCCTGAGC
CACGTTGCGGACATCGATACCATTCAGGACGATGCGATGCGTCACGAATG
CCTGCGTCACGCGATCACCCTGGGTCGTGCGCTGATTGTTCTGGCGGAGC
GTATCGACAGCGTGGATGAACTGCTGGCGCGTGTTCGTCAATACAGCTAA

In specific occurrences of this first instance, the at least one S-methyl-5-thioribose kinase enzyme comprises the amino acid sequence as set forth below in SEQ ID NO: 3.

(SEQ ID NO: 3)
MSQYHTFTAHDAVAYAQQFAGIDNPSELVSAQEVGDGNLNLVFKVFDRQG
VSRAIVKQALPYVRCVGESWPLTLDRARHEAQTLVAHYQHSPQHTVKIHH
FDPELAVMVMEDLSDHRIWRGELIANVYYPQAARQLGDYLAQVLFHTSDF
YLHPHEKKAQVAQFINPAMCEITEDLFFNDPYQIHERNNYPAELEADVAA
LRDDAQLKLAVAALKHRFFAHAEALLHGDIHSGSIFVAEGSLKAIDAEFG
YFGPIGFDIGTAIGNLLLNYCGLPGQLGIRDAAAAREQRLNDIHQLWTTF
AERFQALAAEKTRDAALAYPGYASAFLKKVWADAVGFCGSELIRRSVGLS
HVADIDTIQDDAMRHECLRHAITLGRALIVLAERIDSVDELLARVRQYSL
EHHHHHH

In specific examples of such occurrences, the S-methyl-5-thioribose kinase enzyme comprising the amino acid sequence as set forth above in SEQ ID NO: 3 may be encoded by the DNA sequence as set forth below in SEQ ID NO: 4.

(SEQ ID NO: 4)
ATGAGCCAGTATCATACCTTCACCGCGCATGATGCGGTGGCGTATGCGCA
GCAATTTGCGGGCATTGATAACCCGAGCGAGCTGGTTAGCGCGCAAGAAG
TTGGTGACGGCAACCTGAACCTGGTGTTCAAGGTTTTTGATCGTCAGGGT
GTGAGCCGTGCGATCGTTAAACAAGCGCTGCCGTACGTGCGTTGCGTTGG
TGAAAGCTGGCCGCTGACCCTGGACCGTGCGCGTCATGAAGCGCAGACCC
TGGTGGCGCACTATCAGCACAGCCCGCAACACACCGTTAAGATCCACCAC
TTCGATCCGGAGCTGGCGGTGATGGTTATGGAAGACCTGAGCGATCACCG
TATTTGGCGTGGTGAGCTGATCGCGAACGTGTACTATCCGCAGGCGGCGC
GTCAACTGGGTGACTACCTGGCGCAGGTTCTGTTCCACACCAGCGATTTT
TATCTGCACCCGCACGAGAAGAAAGCGCAGGTGGCGCAATTCATTAACCC
GGCGATGTGCGAGATCACCGAAGACCTGTTCTTTAACGATCCGTACCAGA
TTCACGAACGTAACAACTATCCGGCGGAGCTGGAAGCGGATGTGGCGGCG
CTGCGTGATGATGCGCAACTGAAGCTGGCGGTTGCGGCGCTGAAACACCG
TTTCTTTGCGCATGCGGAGGCGCTGCTGCATGGTGACATTCACAGCGGCA
GCATCTTCGTTGCGGAGGGTAGCCTGAAGGCGATCGACGCGGAATTCGGT
TACTTTGGCCCGATCGGTTTTGATATTGGTACCGCGATCGGCAACCTGCT
GCTGAACTATTGCGGTCTGCCGGGTCAACTGGGTATTCGTGATGCGGCGG
CGGCGCGTGAACAGCGTCTGAACGATATCCACCAACTGTGGACCACCTTC
GCGGAGCGTTTTCAAGCGCTGGCGGCGGAAAAGACCCGTGACGCGGCGCT
GGCGTACCCGGGTTATGCGAGCGCGTTCCTGAAGAAAGTGTGGGCGGATG
CGGTTGGTTTTTGCGGCAGCGAGCTGATTCGTCGTAGCGTGGGCCTGAGC
CACGTTGCGGACATCGATACCATTCAGGACGATGCGATGCGTCACGAATG
CCTGCGTCACGCGATCACCCTGGGTCGTGCGCTGATTGTTCTGGCGGAGC
GTATCGACAGCGTGGATGAACTGCTGGCGCGTGTTCGTCAATACAGCCTC
GAGCACCACCACCACCACCACTGA

In specific occurrences of this first instance, the at least one S-methyl-5-thioribose kinase enzyme comprises the amino acid sequence as set forth below in SEQ ID NO: 5.

MSQYHTFTAHDAVAYAQQFAGIDNPSELVSAQEVGDGNLNLVFKVFDRQG
VSRAIVKQALPYPRCVGESWPLTLDRARHEAQTLVAHYQHSPQHTVKIHH
FDPELAVMVMEDLSDHRIWRGELIANVYYPQAARQLGDYLAQVLFHTSDF
YLHPHEKKAQVAQFINPAMCEISEDLIFNDPYQIHERNNYPAELEADVAA
LRDDAQLKLAVAALKHRFFAHAEALLHGDLHSGSIFVAEGSLKAIDAEFG
YFGPIGFDIGTAIGNLLLNYCGLPGQLGIRDAAAAREQRLNDIHQLWTTF
AERFQALAAEKTRDAALAYPGYASAFLKKVWADAVGFCGSELIRRSVGLS
HVADIDTIQDDAMRHECLRHAITLGRALIVLAERIDSVDELLARVRQYSL
EHHHHHH

In specific examples of such occurrences, the S-methyl-5-thioribose kinase enzyme comprising the amino acid sequence as set forth above in SEQ ID NO: 5 may be encoded by the DNA sequence as set forth below in SEQ ID NO: 6.

(SEQ ID NO: 6)
ATGAGCCAGTATCATACCTTCACCGCGCATGATGCGGTGGCGTATGCGCA
GCAATTTGCGGGCATTGATAACCCGAGCGAGCTGGTTAGCGCGCAAGAAG
TTGGTGACGGCAACCTGAACCTGGTGTTCAAGGTTTTTGATCGTCAGGGT
GTGAGCCGTGCGATCGTTAAACAAGCGCTGCCGTACCCCCGTTGCGTTGG
TGAAAGCTGGCCGCTGACCCTGGACCGTGCGCGTCATGAAGCGCAGACCC
TGGTGGCGCACTATCAGCACAGCCCGCAACACACCGTTAAGATCCACCAC
TTCGATCCGGAGCTGGCGGTGATGGTTATGGAAGACCTGAGCGATCACCG
TATTTGGCGTGGTGAGCTGATCGCGAACGTGTACTATCCGCAGGCGGCGC
GTCAACTGGGTGACTACCTGGCGCAGGTTCTGTTCCACACCAGCGATTTT
TATCTGCACCCGCACGAGAAGAAAGCGCAGGTGGCGCAATTCATTAACCC
GGCGATGTGCGAGATCAGCGAAGACCTGATTTTTAACGATCCGTACCAGA
TTCACGAACGTAACAACTATCCGGCGGAGCTGGAAGCGGATGTGGCGGCG
CTGCGTGATGATGCGCAACTGAAGCTGGCGGTTGCGGCGCTGAAACACCG
TTTCTTTGCGCATGCGGAGGCGCTGCTGCATGGTGACCTTCACAGCGGCA
GCATCTTCGTTGCGGAGGGTAGCCTGAAGGCGATCGACGCGGAATTCGGT
TACTTTGGCCCGATCGGTTTTGATATTGGTACCGCGATCGGCAACCTGCT
GCTGAACTATTGCGGTCTGCCGGGTCAACTGGGTATTCGTGATGCGGCGG
CGGCGCGTGAACAGCGTCTGAACGATATCCACCAACTGTGGACCACCTTC
GCGGAGCGTTTTCAAGCGCTGGCGGCGGAAAAGACCCGTGACGCGGCGCT
GGCGTACCCGGGTTATGCGAGCGCGTTCCTGAAGAAAGTGTGGGCGGATG
CGGTTGGTTTTTGCGGCAGCGAGCTGATTCGTCGTAGCGTGGGCCTGAGC
CACGTTGCGGACATCGATACCATTCAGGACGATGCGATGCGTCACGAATG
CCTGCGTCACGCGATCACCCTGGGTCGTGCGCTGATTGTTCTGGCGGAGC
GTATCGACAGCGTGGATGAACTGCTGGCGCGTGTTCGTCAATACAGCCTC
GAGCACCACCACCACCACCACTGA

In specific occurrences of this instance, the at least one S-methyl-5-thioribose kinase enzyme comprises the amino acid sequence as set forth below in SEQ ID NO: 7.

(SEQ ID NO: 7)
MSQYHTFTADDAVAYAQQFAGIDNPSELVSAQEVGDGNLNLVFKVFDRQG
VSRAIVKQALPYPRRVGPSWPLTLDRARHEAQTLVAHYQHSPQHTVKIHH
FDPELAVMVMEDLSDHRIWRGELIANVYYPQAARQLGDYLAQVLFHTSDF
YLHPHEKKAQVAQFINPAMCEISEDLVFNDPYQIHERNNYPAELEADVAA
LRDDAQLKLAVAALKHRFFAHAEALLHGDLHTGSIFVAEGSLKVIDAEFG
YFGPIGFDIGTAIGNLLLNYCGLPGQLGIRDAAAAREQRLNDIHQLWTTF
AERFQALAAEKTRDAALAYPGYASAFLKKVWADAVGFCGSELIRRSVGLA
HVADIDTIQDDAMRHECLREAITLGRALIVLAERIDSVDELLARVRQYSL
EHHHHHH

In specific examples of such occurrences, the S-methyl-5-thioribose kinase enzyme comprising the amino acid sequence as set forth above in SEQ ID NO: 7 may be encoded by the DNA sequence as set forth below in SEQ ID NO: 8.

(SEQ ID NO: 8)
ATGAGCCAGTATCATACCTTCACCGCGGATGATGCGGTGGCGTATGCGCA
GCAATTTGCGGGCATTGATAACCCGAGCGAGCTGGTTAGCGCGCAAGAAG
TTGGTGACGGCAACCTGAACCTGGTGTTCAAGGTTTTTGATCGTCAGGGT
GTGAGCCGTGCGATCGTTAAACAAGCGCTGCCGTACCCCCGTCGCGTTGG
TCCGAGCTGGCCGCTGACCCTGGACCGTGCCCGTCATGAAGCGCAGACCC
TGGTTGCGCACTATCAGCACAGCCCGCAACACACCGTTAAGATCCACCAC
TTCGATCCGGAGCTGGCGGTGATGGTTATGGAAGACCTGAGCGATCACCG
TATTTGGCGTGGTGAGCTGATCGCGAACGTGTACTATCCGCAGGCGGCGC
GTCAACTGGGTGACTACCTGGCGCAGGTTCTGTTCCACACCAGCGATTTT
TATCTGCACCCGCACGAGAAGAAAGCGCAGGTGGCGCAATTCATTAACCC
GGCCATGTGCGAAATCAGCGAAGACCTGGTGTTTAACGATCCGTACCAGA
TTCACGAACGTAACAACTATCCGGCGGAGCTGGAAGCGGATGTGGCGGCG
CTGCGTGATGATGCGCAACTGAAGCTGGCGGTTGCGGCGCTGAAACACCG
TTTCTTTGCGCATGCGGAGGCGCTGCTGCATGGTGACCTTCACACCGGCA
GCATCTTCGTTGCGGAGGGTAGCCTGAAGGTGATCGACGCGGAATTCGGT
TACTTTGGCCCGATCGGTTTTGATATTGGTACCGCGATCGGCAACCTGCT
GCTGAACTATTGCGGTCTGCCGGGTCAACTGGGTATTCGTGATGCGGCGG
CGGCGCGTGAACAGCGTCTGAACGATATCCACCAACTGTGGACCACCTTC
GCGGAGCGTTTTCAAGCGCTGGCGGCGGAAAAGACCCGTGACGCGGCGCT
GGCGTACCCGGGTTATGCGAGCGCGTTCCTGAAGAAAGTGTGGGCGGATG
CGGTTGGTTTTTGCGGCAGCGAGCTGATTCGTCGTAGCGTGGGCCTGGCG
CACGTTGCGGACATCGATACCATTCAGGACGATGCGATGCGTCACGAATG
CCTGCGTGAAGCGATCACCCTGGGTCGTGCGCTGATTGTTCTGGCGGAGC
GCATCGACAGCGTGGATGAACTGCTGGCGCGTGTTCGTCAATACAGCCTC
GAGCACCACCACCACCACCACTGA

In specific occurrences of this first instance, the at least one S-methyl-5-thioribose kinase enzyme comprises the amino acid sequence as set forth below in SEQ ID NO: 9.

(SEQ ID NO: 9)
MSQYHTFTADDAVAYAQQFAGIDNPSELVSAQEVGDGNLNLVFKVFDRQG
VSRAIVKQALPYPRAVGPSWPLTLDRARHEAQTLVAHYQHSPQHTVKIHH
FDPELAVMVMEDLSDHRIWRGELIANVYYPQAARQLGDYLAQVLFHTSDF
YLHPHEKKAQVAQFINPGMCEISEDLSFNDPYQIHERNNYPAELEADVAA
LRDDAQLKLAVAALKHRFFAHAEALLHGDLHSGSIFVAEGSLKVIDAEFG
YFGPIGFDIGTAIGNLLLNYCGLPGQLGIRDAAAAREQRLNDIHQLWTTF
AERFQALAAEKTRDAALAYPGYASAFLKKVWADAVGFCGSELIRRSVGLS
HVADIDTIQDDAMRHECLRHAITLGRALIVLAETIDSVDELLARVRQYSL
EHHHHHH

In specific examples of such occurrences, the S-methyl-5-thioribose kinase enzyme comprising the amino acid sequence as set forth above in SEQ ID NO: 9 may be encoded by the DNA sequence as set forth below in SEQ ID NO: 10.

(SEQ ID NO: 10)
ATGAGCCAGTATCATACCTTCACCGCGGATGATGCGGTGGCGTATGCGC
AGCAATTTGCGGGCATTGATAACCCGAGCGAGCTGGTTAGCGCGCAAGA
AGTTGGTGACGGCAACCTGAACCTGGTGTTCAAGGTTTTTGATCGTCAG
GGTGTGAGCCGTGCGATCGTTAAACAAGCGCTGCCGTACCCCCGTGCGG
TTGGTCCGAGCTGGCCGCTGACCCTGGACCGTGCGCGTCATGAAGCGCA
GACCCTGGTGGCGCACTATCAGCACAGCCCGCAACACACCGTTAAGATC
CACCACTTCGATCCGGAGCTGGCGGTGATGGTTATGGAAGACCTGAGCG
ATCACCGTATTTGGCGTGGTGAGCTGATCGCGAACGTGTACTATCCGCA
GGCGGCGCGTCAACTGGGTGACTACCTGGCGCAGGTTCTGTTCCACACC
AGCGATTTTTATCTGCACCCGCACGAGAAGAAAGCGCAGGTGGCGCAAT
TCATTAACCCGGGCATGTGCGAAATCAGCGAAGACCTGAGCTTTAACGA
TCCGTACCAGATTCACGAACGTAACAACTATCCGGCGGAGCTGGAAGCG
GATGTGGCGGCGCTGCGTGATGATGCGCAACTGAAGCTGGCGGTTGCGG
CGCTGAAACACCGTTTCTTTGCGCATGCGGAGGCGCTGCTGCATGGTGA
CCTTCACAGCGGCAGCATCTTCGTTGCGGAGGGTAGCCTGAAGGTGATC
GACGCGGAATTCGGTTACTTTGGCCCGATCGGTTTTGATATTGGTACCG
CGATCGGCAACCTGCTGCTGAACTATTGCGGTCTGCCGGGTCAACTGGG
TATTCGTGATGCGGCGGCGGCGCGTGAACAGCGTCTGAACGATATCCAC
CAACTGTGGACCACCTTCGCGGAGCGTTTTCAAGCGCTGGCGGCGGAAA
AGACCCGTGACGCGGCGCTGGCGTACCCGGGTTATGCGAGCGCGTTCCT
GAAGAAAGTGTGGGCGGATGCGGTTGGTTTTTGCGGCAGCGAGCTGATT
CGTCGTAGCGTGGGCCTGTCGCACGTTGCGGACATCGATACCATTCAGG
ACGATGCGATGCGTCACGAATGCCTGCGTCACGCGATCACCCTGGGTCG
TGCGCTGATTGTTCTGGCGGAGACCATCGACAGCGTGGATGAACTGCTG
GCGCGTGTTCGTCAATACAGCCTCGAGCACCACCACCACCACCACTGA

In specific occurrences of this first instance, the at least one S-methyl-5-thioribose kinase enzyme comprises the amino acid sequence as set forth below in SEQ ID NO: 11.

(SEQ ID NO: 11)
MSQYHTFTADDAVAYAQQFAGIDNPSELVSAQEVGDGNLNLVFKVFDRQ
GVSRAIVKQALPYPRRVGPSWPLTLDRARHEAQTLVAHYQHSPQHTVKI
FHFDPELAVMVMEDLSDHRIWRGELIANVYYPQAARQLGDYLAQVLFHT
SDFYLHPHEKKRQVAQFINPAMCGISEDLVENDPYQIHERNNYPAELEA
QVAALRDDAQLKLAVAALKHRFFAHAEALLHGDLHTGSIFVKEGSLKVI
DAEFGYFGPIGFDIGTAIGNLLLNYCGLPGQLGIRDAAAAREQRLNDIH
QLWTTFAERFQALAAEKTRDAALAYPGYASAFLKKVWADAVGFCGSELI
RRSVGLAHVADIDTIQDDAMRHECLREAITLGRALIVLAERIDSVDELL
ARVRQYSLEHHHHHH

In specific occurrences of this first instance, the at least one S-methyl-5-thioribose kinase enzyme comprises the amino acid sequence as set forth below in SEQ ID NO: 12.

(SEQ ID NO: 12)
MSQYHTFTADDAVAYAQQFAGIDNPSELVSAQEVGDGNLNLVFKVFDRQ
GVSRSIVKQALPYPRRVGPSWPLTLDRARHEAQTLVAHYQHSPQHTVKI
FHFDPELAVMVMEDLSDHRIWRGELIANVYYPQAARQLGDYLAQVLFHT
SDFYLHPHEKKRQVAQFINPAMCGISEDLVFNDPYQIHERNNYPAELEA
QVAALRDDAQLKLAVAALKHRFFAHAEALLHGDLHTGSIFVKEGSLKVI
DAEFGYFGPIGFDIGTAIGNLLLNYCGLPGQLGIRDAAAAREQRLNDIH
QLWTTFAERFQALAAEKTRDAALRYPGYASAFLKKVWADAVGFCGSELI
RRSVGLAHVADIDTIQDDAMRHECLREAITLGRALIVLAERIDSVDELL
ARVRQYSLEHHHHHH

In specific occurrences of this first instance, the at least one S-methyl-5-thioribose kinase enzyme comprises the amino acid sequence as set forth below in SEQ ID NO: 13.

(SEQ ID NO: 13)
MGVTKTPLYETLNESSAVALAVKLGLFPSKSTLTCQEIGDGNLNYVFHI
YDQEHDRALIIKQAVPYAKVVGESWPLTIDRARIESSALIRQGEHVPHL
VPRVFYSDTEMAVTVMEDLSHLKIARKGLIEGENYPHLSQHIGEFLGKT
LFYSSDYALEPKVKKQLVKQFTNPELCDITERLVFTDPFFDHDTNDFEE
ELRPFVEKLWNNDSVKIEAAKLKKSFLTSAETLIHGDLHTGSIFASEHE
TKVIDPEFAFYGPIGFDVGQFIANLFLNALSRDGADREPLYEHVNQVWE
TFEETFSEAWQKDSLDVYANIDGYLTDTLSHIFEEAIGFAGCELIRRTI
GLAHVADLDTIVPFDKRIGRKRLALETGTAFIEKRSEFKTITDVIELFK
LLVKE

In some occurrences, the at least one S-methyl-5-thioribose kinase enzyme is a S-methyl-5-thioribose kinase enzyme based on the amino acid sequences of SEQ ID NO: 1, 3, 5, 7, 9, 11, 12, or 13 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 1, 3, 5, 7, 9, 11, 12, or 13. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one S-methyl-5-thioribose kinase enzyme is a S-methyl-5-thioribose kinase enzyme encoded by the DNA sequences of SEQ ID NO: 2, 4, 6, 8, or 10 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 2, 4, 6, 8, or 10. These differences can be amino acid insertions, deletions, substitutions, or any combinations of such changes. In some occurrences, the sequence differences can comprise non-conservative, conservative, as well as a combination of non-conservative and conservative amino acid substitutions.

In a second instance of the second aspect of this second embodiment, the at least one acetate kinase enzyme is one or more acetate kinase enzymes selected from the group consisting of wild-type acetate kinase enzymes and acetate kinase enzymes that are produced from the directed evolution from a commercially available, wild-type acetate kinase enzyme. In specific instances, the at least one acetate kinase enzyme is selected from the group consisting of acetate kinase enzymes that are produced from the directed evolution from a commercially available, wild-type acetate kinase enzyme, which has the amino acid sequence as set forth below in SEQ ID NO: 14.

(SEQ ID NO: 14)
MRVLVINSGSSSIKYQLIEMEGEKVLCKGIAERIGIEGSRLVHRVGDEK
HVIERELPDHEEALKLILNTLVDEKLGVIKDLKEIDAVGHRVVHGGERF
KESVLVDEEVLKAIEEVSPLAPLHNPANLMGIKAAMKLLPGVPNVAVFD
TAFHQTIPQKAYLYAIPYEYYEKYKIRRYGFHGTSHRYVSKRAAEILGK
KLEELKIITCHIGNGASVAAVKYGKCVDTSMGFTPLEGLVMGTRSGDLD
PAIPFFIMEKEGISPQEMYDILNKKSGVYGLSKGFSSDMRDIEEAALKG
DEWCKLVLEIYDYRIAKYIGAYAAAMNGVDAIVFTAGVGENSPITREDV
CSYLEFLGVKLDKQKNEETIRGKEGIISTPDSRVKVLVVPTNEELMIAR
DTKEIVEKIGR

In occurrences, the at least one acetate kinase enzyme is a wild-type acetate kinase enzyme having the amino acid sequence as set forth above in SEQ ID NO: 14. In specific examples of such occurrences, the wild-type acetate kinase enzyme comprising the amino acid sequence as set forth above in SEQ ID NO: 14 may be encoded by the DNA sequence as set forth below in SEQ ID NO: 15.

(SEQ ID NO: 15)
ATGCGTGTTCTGGTTATTAATAGCGGTAGCAGCAGCATTAAATATCAGC
TGATTGAAATGGAAGGTGAAAAAGTTCTGTGTAAAGGTATTGCAGAACG
TATTGGTATTGAAGGTAGCCGTCTGGTTCATCGTGTTGGTGATGAAAAA
CATGTTATTGAACGTGAACTGCCGGATCATGAAGAAGCACTGAAACTGA
TTCTGAATACCCTGGTTGATGAAAAACTGGGTGTTATTAAAGATCTGAA
AGAAATTGACGCAGTTGGTCATCGTGTTGTTCATGGTGGTGAACGTTTT
AAAGAAAGCGTTCTGGTTGATGAAGAAGTTCTGAAAGCAATTGAAGAAG
TTAGCCCGCTGGCACCGCTGCATAATCCGGCAAATCTGATGGGTATTAA
AGCAGCAATGAAACTGCTGCCGGGTGTTCCGAATGTTGCAGTTTTTGAT
ACCGCATTTCATCAGACCATTCCGCAGAAAGCATATCTGTATGCAATTC
CGTATGAATATTACGAAAAATACAAAATTCGTCGCTACGGTTTTCATGG
TACCAGCCATCGTTATGTTAGCAAACGTGCAGCAGAAATTCTGGGTAAA
AAACTGGAAGAACTGAAAATTATCACCTGTCATATTGGTAATGGTGCAA
GCGTTGCAGCAGTTAAATATGGTAAATGTGTTGATACCAGCATGGGTTT
TACCCCGCTGGAAGGTCTGGTTATGGGTACCCGTAGCGGTGATCTGGAT
CCGGCAATTCCGTTTTTTATTATGGAAAAAGAGGGTATTAGCCCGCAGG
AAATGTATGATATTCTGAATAAAAAAAGCGGCGTTTATGGTCTGAGCAA
AGGTTTTAGCAGCGATATGCGTGATATTGAAGAAGCAGCACTGAAAGGT
GATGAATGGTGTAAACTGGTTCTGGAAATTTATGATTACCGTATTGCAA
AATACATCGGTGCATACGCTGCAGCAATGAATGGTGTTGATGCAATTGT
TTTTACCGCAGGTGTTGGTGAAAATAGCCCGATTACCCGTGAAGATGTT
TGTAGCTATCTGGAATTTCTGGGTGTTAAACTGGATAAACAGAAAAATG
AAGAGACCATTCGTGGTAAAGAAGGTATTATTAGCACCCCGGATAGCCG
TGTTAAAGTTCTGGTTGTTCCGACCAATGAAGAACTGATGATTGCACGT
GATACCAAAGAAATTGTTGAAAAAATCGGTCGTTAA

In specific occurrences of this second instance, the at least one acetate kinase enzyme comprises the amino acid sequence as set forth below in SEQ ID NO: 16.

(SEQ ID NO: 16)
MGSHHHHHHGSRVLNINSGSSSIKYQLIEMEGEKVLCKGIAERIGIEGS
RLVHRVGDEKHVIERELPDHEEALKLILNTLVDEKLGVIKDLKEIDAVG
HRVVHGGERFKESVLVDEEVLKAIEEVSPLAPLHNPANLMGIKAAMKLL
PGVPNVQVFDTAFHQTIPQKAYLYAIPYEYYEKYKIRRYGFHGISHRYV
SKRAAEILGKKLEELKIITCHIGNGASVAAVKYGKCVDTSMGFTPLEGL
VMGTRSGDLDPAIPFFIMEKEGISPQEMYDILNKKSGVYGLSKGFSSDM
RDNFEAALKGDEWCKLVLEIYDYRIAKYIGAYAAAMNGVDAIVFTAGVG
ENSPITREDVCKYLEFLGVKLDKQKNEETILGKEGIISTPDSRVKVLVV
PTNEELMIARDTKEIVEKIGR

In specific examples of such occurrences, the acetate kinase enzyme comprising the amino acid sequence as set forth above in SEQ ID NO: 16 may be encoded by the DNA sequence as set forth below in SEQ ID NO: 17.

(SEQ ID NO: 17)
ATGGGTAGCCATCATCATCATCATCACGGTAGCCGTGTTCTGAATATCA
ATAGCGGTAGCAGCAGCATTAAATATCAGCTGATTGAAATGGAAGGTGA
AAAAGTTCTGTGTAAAGGTATTGCAGAACGTATTGGTATTGAAGGTAGC
CGTCTGGTTCATCGTGTTGGTGATGAAAAACATGTTATTGAACGTGAAC
TGCCGGATCATGAAGAAGCACTGAAACTGATTCTGAATACCCTGGTTGA
TGAAAAACTGGGTGTTATTAAAGATCTGAAAGAAATTGACGCAGTTGGT
CATCGTGTTGTTCATGGTGGTGAACGTTTTAAAGAAAGCGTTCTGGTTG
ATGAAGAAGTTCTGAAAGCAATTGAAGAAGTTAGCCCGCTGGCACCGCT
GCATAATCCGGCAAATCTGATGGGTATTAAAGCAGCAATGAAACTGCTG
CCGGGTGTTCCGAATGTTCAAGTTTTTGATACCGCATTTCATCAGACCA
TTCCGCAGAAAGCATATCTGTATGCAATTCCGTATGAATATTACGAAAA
ATACAAAATTCGTCGCTACGGTTTTCATGGTATCAGCCATCGTTATGTT
AGCAAACGTGCAGCAGAAATTCTGGGTAAAAAACTGGAAGAACTGAAAA
TTATCACCTGTCATATTGGTAATGGTGCAAGCGTTGCAGCAGTTAAATA
TGGTAAATGTGTTGATACCAGCATGGGTTTTACCCCGCTGGAAGGTCTG
GTTATGGGTACCCGTAGCGGTGATCTGGATCCGGCAATTCCGTTTTTTA
TTATGGAAAAAGAGGGTATTAGCCCGCAGGAAATGTATGATATTCTGAA
TAAAAAAAGCGGCGTTTATGGTCTGAGCAAAGGTTTTAGCAGCGATATG
CGTGATAATTTTGAAGCAGCACTGAAAGGTGATGAATGGTGTAAACTGG
TTCTGGAAATTTATGACTACCGTATTGCAAAATACATCGGTGCATACGC
TGCAGCAATGAATGGTGTTGATGCAATTGTTTTTACCGCAGGTGTTGGT
GAAAATAGCCCGATCACCCGTGAAGATGTTTGTAAGTATCTGGAATTTC
TGGGTGTTAAACTGGATAAACAGAAAAATGAAGAGACTATTCTGGGTAA
AGAAGGTATTATTAGCACCCCGGATAGCCGTGTTAAAGTTCTGGTTGTT
CCGACCAATGAAGAACTGATGATTGCACGTGATACCAAAGAAATTGTTG
AAAAAATCGGTCGTTAA

In some occurrences of this second instance, the at least one acetate kinase enzyme is an acetate kinase enzyme based on the amino acid sequences of SEQ ID NO: 14 or 16 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 14 or 16. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences of this second instance, the at least one acetate kinase enzyme is an acetate kinase enzyme encoded by the DNA sequences of SEQ ID NO: 15 or 17 and can comprise an amino acid sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 15 or 17. These differences can be amino acid insertions, deletions, substitutions, or any combinations of such changes. In some occurrences, the sequence differences can comprise non-conservative, conservative, as well as a combination of non-conservative and conservative amino acid substitutions.

In a third instance of the second aspect of this second embodiment, the at least one pyruvate oxidase enzyme is one or more pyruvate oxidase enzymes selected from the group consisting of wild-type pyruvate oxidase enzymes and pyruvate oxidase enzymes that are produced from the directed evolution from a wild-type pyruvate oxidase enzyme. In specific instances, the at least one pyruvate oxidase enzyme is a wild-type pyruvate oxidase enzyme having the amino acid sequence as set forth below in SEQ ID NO: 18.

(SEQ ID NO: 18)
MSDNKINIGLAVMKILESWGADTIYGIPSGTLSSLMDAMGEEENNVKFL
QVKHEEVGAMAAVMQSKFGGNLGVTVGSGGPGASHLINGLYDAAMDNIP
VVAILGSRPQRELNMDAFQELNQNPMYDHIAVYNRRVAYAEQLPKLVDE
AARMAIAKRGVAVLEVPGDFAKVEIDNDQWYSSANSLRKYEPIAPAAQD
IDAAVELLNNSKRPVIYAGIGTMGHGPAVQELARKIKAPVITTGKNFET
FEWDFEALTGSTYRVGWKPANETILEADTVLFAGSNFPFSEVEGTFRNV
DNFIQIDIDPAMLGKRHHADVAILGDAGLAIDEILNKVDAVEESAWWTA
NLKNIANWREYINMLETKEEGDLQFYQVYNAINNHADEDAIYSIDVGNS
TQTSIRHLHMTPKNMWRTSPLFATMGIAIPGGLGAKNTYPDRQVWNIIG
DGAFSMTYPDVVTNVRYNMPVINVVFSNTEYAFIKNKYEDTNKNLFGVD
FTDVDYAKIAEAQGAKGFTVSRIEDMDRVMAEAVAANKAGHTVVIDCKI
TQDRPIPVETLKLDSKLYSEDEIKAYKERYEAANLVPFREYLEAEGLES
KYIK

In specific occurrences, the wild-type pyruvate oxidase enzyme comprising the amino acid sequence as set forth above in SEQ ID NO: 18 may be encoded by the DNA sequence as set forth below in SEQ ID NO: 19.

(SEQ ID NO: 19)
ATGAGTGATAATAAAATCAACATCGGTCTGGCGGTGATGAAAATTCTGG
AAAGTTGGGGCGCAGATACCATTTATGGCATTCCGAGCGGCACCCTGAG
TAGCTTAATGGATGCCATGGGTGAAGAAGAAAACAATGTTAAGTTCCTG
CAGGTGAAGCATGAAGAAGTGGGCGCAATGGCAGCCGTTATGCAGAGTA
AATTTGGCGGTAATCTGGGTGTGACCGTTGGTAGTGGCGGTCCTGGTGC
AAGTCATCTGATTAATGGCCTGTATGATGCCGCAATGGATAATATTCCG
GTTGTGGCAATTCTGGGTAGCCGTCCTCAGCGTGAACTGAATATGGATG
CATTTCAGGAACTGAACCAGAATCCGATGTATGATCATATCGCCGTTTA
CAATCGCCGTGTGGCCTATGCAGAACAGCTGCCTAAACTGGTGGATGAA
GCCGCCCGTATGGCCATTGCTAAACGCGGTGTGGCAGTTCTGGAAGTTC
CGGGTGATTTTGCAAAAGTGGAAATTGATAACGACCAGTGGTACAGTAG
CGCAAATAGCCTGCGCAAATATGAACCGATTGCCCCGGCAGCACAGGAT
ATTGATGCAGCTGTGGAACTGCTGAATAATAGTAAACGCCCGGTGATTT
ACGCAGGTATTGGTACCATGGGCCATGGCCCTGCAGTTCAAGAACTGGC
TCGTAAAATTAAGGCACCGGTGATTACCACCGGTAAAAATTTTGAAACC
TTCGAGTGGGACTTCGAAGCCCTGACCGGTTCAACCTATCGTGTGGGTT
GGAAACCGGCAAATGAAACCATTCTGGAAGCCGATACCGTTCTGTTTGC
AGGCAGCAATTTTCCGTTTAGCGAAGTGGAAGGCACCTTTCGTAATGTT
GATAATTTCATCCAGATCGACATCGACCCGGCAATGCTGGGTAAACGCC
ATCATGCAGATGTGGCAATTCTGGGTGATGCCGGCCTGGCAATTGATGA
AATTCTGAATAAGGTGGACGCCGTGGAAGAAAGCGCCTGGTGGACCGCA
AATCTGAAAAATATTGCAAACTGGCGCGAGTATATCAACATGCTGGAAA
CCAAAGAGGAGGGTGATCTGCAGTTTTATCAGGTTTATAACGCGATCAA
CAACCACGCCGATGAAGATGCAATTTATAGCATTGACGTGGGCAATAGC
ACCCAGACCAGCATTCGTCATCTGCACATGACCCCGAAAAATATGTGGC
GCACCAGCCCGCTGTTTGCCACCATGGGTATTGCAATTCCGGGTGGCCT
GGGTGCAAAAAATACCTATCCGGATCGTCAGGTGTGGAATATTATTGGC
GATGGCGCCTTTAGTATGACCTATCCGGATGTTGTTACCAATGTTCGCT
ATAATATGCCGGTTATCAACGTGGTTTTCAGTAATACCGAGTACGCATT
TATCAAGAACAAGTACGAGGACACCAACAAAAACCTGTTTGGTGTGGAT
TTCACCGATGTGGATTATGCCAAAATCGCCGAAGCACAGGGTGCAAAAG
GCTTTACCGTGAGTCGCATTGAAGATATGGATCGTGTTATGGCCGAAGC
CGTGGCCGCAAATAAAGCCGGTCATACCGTTGTGATTGATTGTAAAATC
ACCCAGGACCGTCCGATTCCGGTTGAAACCCTGAAACTGGATAGCAAAC
TGTATAGTGAGGACGAAATCAAGGCATATAAGGAACGTTACGAGGCAGC
AAATCTGGTGCCGTTTCGTGAATATCTGGAAGCCGAAGGTCTGGAAAGC
AAATATATTAAGTAA

In specific occurrences of this third instance, the at least one pyruvate oxidase enzyme is selected from a wild-type pyruvate oxidase enzyme having the amino acid sequence as set forth below in SEQ ID NO: 20.

(SEQ ID NO: 20)
MTQGKITASAAMLNVLKTWGVDTIYGIPSGTLSSLMDALAEDKDIRFLQ
VRHEETGALAAVMQAKFGGSIGVAVGSGGPGATHLINGVYDAAMDNTPF
LAILGSRPVNELNMDAFQELNQNPMYHGIAVYNKRVAYAEQLPKVIDEA
CRAAVSKKGPAVVEIPVNFGFQEIDENSYYGSGSYERSFIAPALNEVEI
DKAVEILNKAERPVIYAGFGGVKAGEVITELSRKIKAPIITTGKNFEAF
EWNYEGLTGSAYRVGWKPANEVVFEADTVLFLGSNFPFAEVYEAFKNTE
KFIQVDIDPYKLGKRHALDASILGDAGQAAKAILDKVDAVESTPWWRAN
VKNNQNWRDYMNKLEGKTEGELQLYQVYNAINKHADQDAIYSIDVGNST
QTSTRHLHMTPKNMWRTSPLFATMGIALPGGIAAKKDNPERQVWNIMGD
GAFNMCYPDVITNVQYNLPVINVVFSNAEYAFIKNKYEDTNKHLFGVDF
TNADYAKIAEAQGAVGFTVDRIEDIDAVVAEAVKLNKEGKTVVIDARIT
QHRPLPVEVLELDPKLHSEEAIKAFKEKYEAEELVPFRLFLEEEGLQSR
AIK

In specific examples of such occurrences, the pyruvate oxidase enzyme comprising the amino acid sequence as set forth above in SEQ ID NO: 20 may be encoded by the DNA sequence as set forth below in SEQ ID NO: 21.

(SEQ ID NO: 21)
ATGACCCAGGGTAAAATTACCGCAAGTGCCGCAATGCTGAATGTGCTGA
AAACCTGGGGCGTTGATACCATTTATGGTATTCCGAGTGGTACCCTGAG
CAGCCTGATGGATGCCTTAGCAGAAGATAAAGATATCCGCTTTCTGCAG
GTGCGCCATGAAGAAACCGGCGCATTAGCCGCAGTGATGCAGGCAAAAT
TTGGTGGCAGCATTGGTGTTGCAGTGGGTAGCGGTGGTCCGGGTGCAAC
CCACCTGATCAATGGTGTTTATGATGCCGCCATGGATAATACCCCGTTT
CTGGCCATTCTGGGTAGTCGCCCTGTGAATGAACTGAATATGGATGCAT
TCCAGGAGCTGAATCAGAATCCGATGTATCATGGTATCGCCGTTTATAA
TAAGCGCGTTGCATACGCTGAACAGCTGCCGAAAGTGATTGATGAAGCA
TGCCGTGCAGCCGTGAGCAAAAAAGGCCCTGCAGTGGTTGAAATTCCGG
TTAATTTTGGCTTCCAGGAAATCGATGAGAACAGTTATTACGGCAGCGG
TAGTTATGAACGTAGCTTTATTGCCCCGGCCCTGAATGAAGTGGAAATT
GATAAAGCAGTGGAGATCCTGAACAAGGCAGAACGCCCGGTGATTTATG
CAGGCTTTGGTGGCGTGAAAGCAGGTGAAGTGATTACCGAACTGAGCCG
CAAAATTAAAGCCCCGATTATTACCACCGGCAAAAATTTTGAGGCCTTT
GAATGGAACTACGAGGGACTGACCGGCAGTGCATATCGTGTGGGTTGGA
AACCGGCAAATGAAGTGGTGTTTGAAGCCGATACCGTGCTGTTTCTGGG
TAGCAATTTTCCGTTTGCCGAAGTTTATGAGGCATTTAAAAACACCGAG
AAGTTCATCCAGGTGGATATTGATCCGTATAAGCTGGGCAAACGTCATG
CACTGGATGCAAGTATTCTGGGTGATGCAGGTCAGGCCGCAAAAGCAAT
TCTGGATAAAGTTGATGCCGTGGAAAGCACCCCGTGGTGGCGTGCAAAT
GTGAAAAATAATCAGAACTGGCGCGACTATATGAACAAACTGGAAGGCA
AAACCGAGGGTGAACTGCAGCTGTATCAGGTTTATAATGCCATTAACAA
GCACGCAGACCAGGATGCAATTTATAGTATTGACGTGGGCAACAGCACC
CAGACCAGTACACGTCATCTGCACATGACCCCGAAAAATATGTGGCGTA
CCAGCCCGCTGTTTGCAACCATGGGTATTGCCCTGCCGGGCGGTATTGC
TGCAAAAAAAGATAATCCGGAGCGTCAGGTTTGGAATATTATGGGTGAT
GGTGCCTTTAACATGTGCTATCCGGATGTGATTACCAATGTTCAGTACA
ATCTGCCGGTTATTAACGTTGTTTTCAGCAATGCCGAGTACGCATTTAT
TAAGAACAAGTACGAGGACACCAACAAGCATCTGTTTGGTGTTGATTTC
ACCAACGCCGATTATGCCAAAATTGCCGAAGCACAGGGCGCAGTTGGTT
TTACCGTGGATCGCATTGAAGATATTGACGCAGTGGTTGCCGAAGCAGT
GAAACTGAATAAAGAAGGTAAGACCGTGGTGATTGACGCCCGCATTACC
CAGCATCGCCCTTTACCTGTGGAAGTGCTGGAACTGGATCCGAAACTGC
ATAGTGAAGAAGCAATTAAGGCCTTTAAGGAGAAGTACGAAGCCGAAGA
ACTGGTGCCGTTTCGTCTGTTTCTGGAAGAAGAAGGCCTGCAGAGTCGC
GCAATTAAATAA

In specific occurrences of this third instance, the at least one pyruvate oxidase enzyme is selected from a wild-type pyruvate oxidase enzyme having the amino acid sequence as set forth below in SEQ ID NO: 22.

(SEQ ID NO: 22)
MSDKKISAGLAALKVMEGWGIDTMYGIPSGTLSGLMNAMGHPENKVKFI
QVKHEEIGAMAAVMQYKFGGNIAVTVGSGGPGATHLINGLYDAYMDHIP
VLAILGSKPVRELNMDSFQELNQNPMYDHIAVYNRRVATAEQLPHLVDD
AIRTAISKRGVAVLEVPADFGFAELDAESIYSTPLYSSGTKYKRYKPVA
PEAEEIDEAVEILKASERPVIYAGFGTQGHGDLVEELSRKIKAPVITTG
KNFDNFNWDFEALTGSAFRVGWKPANEAVLEADTVLFIGTNFPFSEVEG
TFRNVKKFIQIDANPDMLGKRHETDVAILADAGESLKALLAKVEPVADT
PWWQANIKNVQNWRDYMNKLEQKTEGPLQAYQVYNAINKLADEDAIFST
DVGDVTQLSTRHLHMNPKQMWRTSPLFATMGIALPGGIGAKNIYPDRQV
WNIIGDGAFSMTYPDVVTSVRYDMPMINVIFTNTEYGFIKNKYEDTNEY
NFGVDFTDVDYAKVGEAQGAIGLTVNRIEDIDRVMQEAVDYYKQGRVVV
VDAKITKDRPIPVETLKLDTNLYSEDVVKAYKEKYEAEALVPFREFLEG
EGLKSIYIKEDNDNKFSF

In specific examples of such occurrences, the pyruvate oxidase enzyme comprising the amino acid sequence as set forth above in SEQ ID NO: 22 may be encoded by the DNA sequence as set forth below in SEQ ID NO: 23.

(SEQ ID NO: 23)
ATGAGTGATAAAAAAATCAGCGCGGGCCTGGCAGCACTGAAAGTTATGG
AAGGTTGGGGTATTGATACCATGTATGGTATTCCGAGCGGTACCCTGAG
CGGTTTAATGAATGCAATGGGCCATCCGGAAAATAAAGTTAAATTCATC
CAGGTGAAGCACGAGGAAATTGGCGCAATGGCAGCCGTTATGCAGTATA
AATTTGGTGGCAATATCGCGGTTACCGTTGGTAGCGGTGGCCCTGGTGC
AACCCACCTGATCAATGGTCTGTATGATGCCTATATGGACCATATTCCG
GTTCTGGCAATTCTGGGTAGTAAACCGGTTCGCGAACTGAATATGGATA
GTTTTCAGGAACTGAACCAGAACCCGATGTATGATCATATTGCCGTTTA
CAACCGCCGCGTTGCAACCGCAGAACAGTTACCTCATCTGGTGGATGAT
GCAATTCGCACCGCCATTAGTAAACGCGGTGTGGCTGTTCTGGAAGTTC
CGGCAGATTTTGGTTTTGCCGAACTGGATGCCGAAAGCATTTATAGCAC
CCCGCTGTATAGTAGTGGCACCAAATATAAACGCTACAAGCCGGTTGCA
CCGGAAGCCGAGGAAATTGATGAAGCCGTGGAAATTCTGAAGGCAAGCG
AACGTCCGGTTATTTATGCCGGTTTTGGTACCCAGGGCCATGGTGATCT
GGTGGAAGAACTGAGTCGCAAAATTAAAGCACCGGTGATTACCACCGGT
AAAAATTTTGATAACTTCAACTGGGACTTCGAGGCACTGACCGGCTCAG
CATTTCGCGTTGGTTGGAAACCGGCCAATGAAGCCGTGCTGGAAGCCGA
CACAGTTCTGTTTATTGGCACCAATTTCCCGTTTAGTGAGGTGGAAGGC
ACCTTTCGCAATGTTAAAAAATTCATCCAGATCGACGCAAACCCGGATA
TGCTGGGTAAACGTCATGAAACCGATGTTGCCATTCTGGCAGATGCCGG
TGAAAGCCTGAAAGCCCTGCTGGCAAAAGTTGAACCGGTGGCAGATACC
CCGTGGTGGCAGGCAAATATTAAAAATGTGCAGAACTGGCGCGACTATA
TGAATAAACTGGAACAGAAGACCGAGGGCCCGCTGCAGGCATACCAGGT
GTACAATGCAATTAATAAGCTGGCCGATGAGGATGCCATTTTTAGTACC
GATGTGGGTGATGTTACCCAGCTGAGCACCCGTCATCTGCACATGAATC
CGAAACAGATGTGGCGTACCAGTCCGCTGTTTGCAACCATGGGCATTGC
CCTGCCGGGTGGTATTGGAGCAAAAAATATTTACCCGGACCGTCAGGTT
TGGAATATTATTGGCGATGGCGCATTTAGCATGACCTATCCGGATGTTG
TTACCAGTGTTCGTTATGATATGCCGATGATTAACGTGATCTTCACCAA
TACCGAGTACGGTTTTATCAAGAACAAGTACGAGGACACCAACGAATAT
AACTTCGGCGTGGATTTTACCGATGTGGATTATGCCAAAGTGGGCGAAG
CCCAGGGCGCAATCGGTCTGACCGTGAACAGAATTGAAGATATTGACCG
CGTGATGCAGGAAGCCGTGGATTATTATAAACAGGGTCGCGTTGTTGTG
GTTGATGCAAAAATTACCAAGGACCGCCCGATTCCGGTTGAAACCCTGA
AACTGGATACCAATCTGTATAGTGAGGACGTTGTTAAGGCCTATAAGGA
AAAATACGAGGCAGAAGCACTGGTTCCGTTTCGCGAATTTCTGGAAGGT
GAAGGTCTGAAAAGTATCTATATCAAGGAGGACAACGACAACAAGTTCA
GTTTTTAA

In specific occurrences of this third instance, the at least one pyruvate oxidase enzyme is the wild-type pyruvate oxidase enzyme that comprises the amino acid sequence as set forth below in SEQ ID NO: 24.

(SEQ ID NO: 24)
MTVGKTKVSTASLKVLAGWGIDTIYGIPSGTLAPLMEALGEQEETDIKF
LQVKHEEVGAMAAVMQWKFGGKLGVCVGSGGPGASHLINGLYDAAMDNT
PVLAILGSPPQRELNMDAFQELNQNPMYDHIAVYNRRVAYAEQLPKLID
DAIRTAISKRGVAVLEVPGDFGYKEIANDAFYSSGHSYRDYVSSAINEV
DIDAAVEVLNKSKRAVIYAGIGTMGHGPAVQELSRKIKAPIITTAKNFE
TFDYDFEGLTGSTYRVGWKPANEAVKEADTVLFVGSNFPFAEVEGTFSN
VENFIQIDNNPTMLGKRHNADVAILGDAGEAVQMLLEKVAPVEESAWWN
ANLKNIQNWRDYMTKLETKENGPLQLYQVYNAINKYADEDAIYSIDVGN
TTQTSIRHLHMTPKNMWRTSPLFASMGIALPGGIGAKNVYPERQVFNLM
GDGAFSMNYQDIVTNVRYNMPVINVVFTNTEYGFIKNKYEDTNTNTFGT
EFTDVDYAMIGEAQGAVGFTVSRIEDMDQVMAAAVKANKEGKTVVIDAK
ITKDRPIPVETLKLDPALYSEEEIKAYKERYEAEELVPFSEFLKAEGLE
SKVAK

In some occurrences, the at least one pyruvate oxidase enzyme is a pyruvate oxidase enzyme based on the amino acid sequences of SEQ ID NO: 18, 20, 22, or 24 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 18, 20, 22, or 24. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one pyruvate oxidase enzyme is a pyruvate oxidase enzyme encoded by the DNA sequences of SEQ ID NO: 19, 21, or 23 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 19, 21, or 23. These differences can be amino acid insertions, deletions, substitutions, or any combinations of such changes. In some occurrences, the sequence differences can comprise non-conservative, conservative, as well as a combination of non-conservative and conservative amino acid substitutions.

In a fourth instance of the second aspect of this second embodiment, the at least one catalase enzyme is selected from the group consisting of wild-type catalase enzymes and catalase enzymes that are produced from the directed evolution from a commercially available, wild-type catalase enzyme, including the catalase enzyme commercially available as product number 11650645103 from Roche Diagnostics International Ltd. In specific occurrences of this fourth instance, the at least one catalase enzyme is the wild-type catalase enzyme that comprises the amino acid sequence as set forth below in SEQ ID NO: 52.

(SEQ ID NO: 52)
MSEKSAADQIVDRGMRPKLSGNTTRHNGAPVPSENISATA
GPQGPNVLNDIHLIEKLAHFNRENVPERIPHAKGHGAFGE
LHITEDVSEYTKADLFQPGKVTPLAVRFSTVAGEQGSPDT
WRDVHGFALRFYTEEGNYDIVGNNTPTFFLRDGMKFPDFI
HSQKRLNKNGLRDADMQWDFWTRAPESAHQVTYLMGDRGT
PKTSRHQDGFGSHTFQWINAEGKPVWVKYHFKTRQGWDCF
TDAEAAKVAGENADYQREDLYNAIENGDFPIWDVKVQIMP
FEDAENYRWNPFDLTKTWSQKDYPLIPVGYFILNRNPRNF
FAQIEQIALDPGNIVPGVGLSPDRMLQARIFAYADQQRYR
IGANYRDLPVNRPINEVNTYSREGSMQYIFDAEGEPSYSP
NRYDKGAGYLDNGTDSSSNHTSYGQADDIYVNPDPHGTDL
VRAAYVKHQDDDDFIQPGILYREVLDEGEKERLADNISNA
MQGISEATEPRVYDYWNNVDENLGARVKELYLQKKA

In specific examples of such occurrences, the catalase enzyme comprising the amino acid sequence as set forth above in SEQ ID NO: 52 may be encoded by the DNA sequence as set forth below in SEQ ID NO: 53.

(SEQ ID NO: 53)
ATGAGCGAAAAGAGCGCGGCGGACCAAATTGTTGATCGTG
GTATGCGTCCGAAACTGAGCGGCAACACCACCCGTCACAA
CGGTGCGCCGGTTCCGAGCGAAAACATCAGCGCGACCGCG
GGTCCGCAGGGTCCGAACGTGCTGAACGACATCCACCTGA
TTGAGAAGCTGGCGCACTTCAACCGTGAAAACGTTCCGGA
GCGTATTCCGCACGCGAAAGGTCACGGCGCGTTTGGTGAA
CTGCACATCACCGAAGACGTGAGCGAGTACACCAAGGCGG
ACCTGTTCCAACCGGGTAAAGTGACCCCGCTGGCGGTTCG
TTTTAGCACCGTTGCGGGCGAGCAAGGTAGCCCGGACACC
TGGCGTGATGTTCACGGTTTCGCGCTGCGTTTTTACACCG
AGGAAGGTAACTACGATATTGTGGGCAACAACACCCCGAC
CTTCTTTCTGCGTGACGGTATGAAGTTCCCGGATTTTATC
CACAGCCAGAAGCGTCTGAACAAAAACGGCCTGCGTGACG
CGGATATGCAGTGGGACTTTTGGACCCGTGCGCCGGAAAG
CGCGCACCAAGTTACCTATCTGATGGGTGACCGTGGTACC
CCGAAGACCAGCCGTCACCAGGATGGTTTCGGCAGCCACA
CCTTTCAATGGATCAACGCGGAGGGCAAACCGGTGTGGGT
TAAGTACCACTTCAAAACCCGTCAGGGTTGGGACTGCTTT
ACCGATGCGGAAGCGGCGAAGGTGGCGGGCGAGAACGCGG
ACTACCAACGTGAAGATCTGTATAACGCGATCGAGAACGG
TGACTTCCCGATTTGGGATGTGAAAGTTCAGATCATGCCG
TTCGAAGATGCGGAGAACTACCGTTGGAACCCGTTTGACC
TGACCAAGACCTGGAGCCAAAAAGATTATCCGCTGATCCC
GGTTGGTTACTTTATTCTGAACCGTAACCCGCGTAACTTC
TTTGCGCAGATCGAACAAATTGCGCTGGACCCGGGCAACA
TTGTGCCGGGTGTTGGTCTGAGCCCGGACCGTATGCTGCA
GGCGCGTATTTTCGCGTACGCGGATCAGCAACGTTATCGT
ATCGGTGCGAACTACCGTGACCTGCCGGTTAACCGTCCGA
TTAACGAAGTGAACACCTATAGCCGTGAGGGCAGCATGCA
ATACATCTTTGATGCGGAGGGTGAACCGAGCTACAGCCCG
AACCGTTATGACAAGGGTGCGGGCTATCTGGACAACGGCA
CCGACAGCAGCAGCAACCACACCAGCTATGGTCAGGCGGA
CGATATCTACGTTAACCCGGACCCGCACGGTACCGATCTG
GTTCGTGCGGCGTATGTGAAGCACCAGGACGATGACGATT
TCATCCAACCGGGCATTCTGTACCGTGAGGTGCTGGACGA
GGGTGAAAAAGAGCGTCTGGCGGATAACATTAGCAACGCG
ATGCAAGGTATCAGCGAAGCGACCGAGCCGCGTGTTTACG
ACTATTGGAACAACGTGGATGAAAACCTGGGCGCGCGTGT
GAAAGAGCTGTACCTGCAGAAGAAAGCGTAA

In specific examples of such occurrences, the catalase enzyme comprising the amino acid sequence as set forth above in SEQ ID NO: 52 may be encoded by the DNA sequence as set forth below in SEQ ID NO: 54.

(SEQ ID NO: 54)
ATGTCCGAAAAATCAGCGGCGGATCAGATTGTAGACCGCG
GAATGCGTCCGAAACTGTCCGGAAACACCACCCGCCACAA
CGGAGCGCCGGTGCCGTCCGAGAACATTAGCGCGACCGCG
GGCCCGCAGGGCCCGAACGTGCTCAATGATATCCATCTCA
TCGAAAAACTCGCGCACTTTAACCGCGAAAACGTGCCGGA
GCGTATTCCTCATGCGAAAGGCCACGGCGCATTCGGTGAA
CTGCATATTACCGAGGATGTAAGCGAATACACCAAAGCGG
ATCTGTTCCAGCCTGGTAAAGTTACCCCCCTGGCAGTGCG
CTTCAGCACAGTGGCAGGTGAACAGGGCAGCCCAGACACC
TGGCGCGATGTTCACGGCTTCGCACTGCGCTTCTACACCG
AAGAGGGCAACTACGATATTGTAGGTAACAACACCCCGAC
CTTCTTCCTGCGTGATGGCATGAAATTCCCCGATTTCATT
CATTCACAGAAACGTCTCAACAAAAACGGTCTGCGCGACG
CGGATATGCAGTGGGACTTCTGGACCCGCGCGCCTGAATC
CGCGCACCAGGTAACCTACCTGATGGGTGATCGCGGTACC
CCTAAAACCAGCCGCCATCAGGATGGCTTCGGCAGCCACA
CCTTCCAGTGGATTAACGCAGAAGGTAAACCGGTGTGGGT
GAAATACCATTTCAAAACCCGCCAGGGCTGGGACTGCTTC
ACCGACGCGGAAGCGGCGAAAGTGGCGGGCGAAAACGCAG
ATTACCAGCGCGAAGATCTCTACAACGCAATCGAAAACGG
CGATTTCCCGATTTGGGATGTTAAAGTGCAGATTATGCCT
TTCGAGGACGCGGAAAACTACCGCTGGAACCCGTTCGACC
TGACCAAAACCTGGTCCCAGAAAGACTACCCGCTGATTCC
GGTTGGTTACTTCATTCTGAACCGCAACCCGCGCAACTTC
TTCGCACAGATTGAGCAGATCGCGCTGGACCCGGGCAACA
TTGTGCCTGGCGTTGGCCTGAGCCCGGATCGCATGCTCCA
GGCGCGTATTTTCGCGTACGCGGATCAGCAGCGTTACCGC
ATTGGCGCAAACTACCGCGATCTGCCGGTAAACCGTCCGA
TTAACGAAGTTAACACCTACTCACGCGAAGGTAGCATGCA
GTACATTTTCGATGCAGAGGGCGAACCTAGCTACTCACCT
AACCGCTACGATAAAGGCGCGGGCTACCTGGATAACGGTA
CTGACAGCAGCAGCAACCACACCAGCTACGGCCAGGCAGA
CGATATTTACGTTAACCCGGATCCGCACGGCACCGATCTG
GTGCGTGCTGCATACGTTAAACACCAGGACGACGATGATT
TCATTCAGCCGGGCATTCTATACCGCGAGGTTCTGGACGA
AGGCGAGAAAGAACGATTGGCGGATAACATTAGCAACGCT
ATGCAGGGCATTTCCGAGGCAACCGAACCGCGCGTTTACG
ATTACTGGAACAACGTGGACGAGAACCTCGGCGCACGCGT
TAAAGAACTGTACCTCCAGAAAAAAGCTTAA

In some occurrences, the at least one catalase enzyme is at least about 85%, 86%, 87%, 88%, 89%, 90%, 91%, 92%, 93%, 94%, 95%, 96%, 97%, 98%, or 99% identical to the reference sequence of the catalase enzyme commercially available as product number 11650645103 from Roche Diagnostics International Ltd. In some occurrences, the at least one catalase enzyme is at least about 85%, 86%, 87%, 88%, 89%, 90%, 91%, 92%, 93%, 94%, 95%, 96%, 97%, 98%, or 99% identical to the reference sequence SEQ ID NO: 52. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one catalase enzyme is encoded by DNA sequences that are at least about 85%, 86%, 87%, 88%, 89%, 90%, 91%, 92%, 93%, 94%, 95%, 96%, 97%, 98%, or 99% identical to the reference sequence of the catalase enzyme commercially available as product number 11650645103 from Roche Diagnostics International Ltd. In some occurrences, the at least one catalase enzyme is encoded by DNA sequences that are at least about 85%, 86%, 87%, 88%, 89%, 90%, 91%, 92%, 93%, 94%, 95%, 96%, 97%, 98%, or 99% identical to SEQ ID NO: 53 or 54. These differences can be amino acid insertions, deletions, substitutions, or any combinations of such changes. In some occurrences, the sequence differences can comprise non-conservative, conservative, as well as a combination of non-conservative and conservative amino acid substitutions.

In a fifth instance of the second aspect of this second embodiment, the at least one uridine phosphorylase enzyme is one or more uridine phosphorylase enzymes selected from the group consisting of wild-type uridine phosphorylase enzymes and uridine phosphorylase enzymes that are produced from the directed evolution from a commercially available, wild-type uridine phosphorylase enzyme. In specific instances, the at least one uridine phosphorylase enzyme is selected from a wild-type uridine phosphorylase enzyme having the amino acid sequence as set forth below in SEQ ID NO: 25.

(SEQ ID NO: 25)
MSKSDVFHLGLTKNDLQGATLAIVPGDPDRVEKIAALMDK
PVKLASHREFTTWRAELDGKPVIVCSTGIGGPSTSIAVEE
LAQLGIRTFLRIGTTGAIQPHINVGDVLVTTASVRLDGAS
LHFAPLEFPAVADFECTTALVEAAKSIGATTHVGVTASSD
TFYPGQERYDTYSGRVVRHFKGSMEEWQAMGVMNYEMESA
TLLTMCASQGLRAGMVAGVIVNRTQQEIPNAETMKQTESH
AVKIVVEAARRLL

In specific occurrences, the wild-type uridine phosphorylase enzyme comprising the amino acid sequence as set forth above in SEQ ID NO: 25 may be encoded by the DNA sequence as set forth below in SEQ ID NO: 26.

(SEQ ID NO: 26)
ATGTCCAAGTCTGATGTTTTTCATCTCGGCCTCACTAAAA
ACGATTTACAAGGGGCTACGCTTGCCATCGTCCCTGGCGA
CCCGGATCGTGTGGAAAAGATCGCCGCGCTGATGGATAAG
CCGGTTAAGCTGGCATCTCACCGCGAATTCACTACCTGGC
GTGCAGAGCTGGATGGTAAACCTGTTATCGTCTGCTCTAC
CGGTATCGGCGGCCCGTCTACCTCTATTGCTGTTGAAGAG
CTGGCACAGCTGGGCATTCGCACCTTCCTGCGTATCGGTA
CAACGGGCGCTATTCAGCCGCATATTAATGTGGGTGATGT
CCTGGTTACCACGGCGTCTGTCCGTCTGGATGGCGCGAGC
CTGCACTTCGCACCGCTGGAATTCCCGGCTGTCGCTGATT
TCGAATGTACGACTGCGCTGGTTGAAGCTGCGAAATCCAT
TGGCGCGACAACTCACGTTGGCGTGACAGCTTCTTCTGAT
ACCTTCTACCCAGGTCAGGAACGTTACGATACTTACTCTG
GTCGCGTAGTTCGTCACTTTAAAGGTTCTATGGAAGAGTG
GCAGGCGATGGGCGTAATGAACTATGAAATGGAATCTGCA
ACCCTGCTGACCATGTGCGCAAGTCAGGGCCTGCGTGCCG
GTATGGTAGCGGGTGTTATCGTTAACCGCACCCAGCAAGA
GATCCCGAATGCTGAGACGATGAAACAAACCGAAAGCCAT
GCGGTGAAAATCGTGGTGGAAGCGGCGCGTCGTCTGCTGT
AA

In specific occurrences of this fifth instance, the at least one uridine phosphorylase enzyme is a wild-type uridine phosphorylase enzyme that comprises the amino acid sequence as set forth below in SEQ ID NO: 27.

(SEQ ID NO: 27)
MHHHHHHGGSGMSKSDVFHLGLTKNDLQGATLAIVPGDPD
RVEKIAALMDKPVKLASHREFTTWRAELDGKPVIVCSTGI
GGPSTSIAVEELAQLGIRTFLRIGTTGAIQPHINVGDVLV
TTASVRLDGASLHFAPLEFPAVADFECTTALVEAAKSIGA
TTHVGVTASSDTFYPGQERYDTYSGRVVRHFKGSMEEWQA
MGVMNYEMESATLLTMCASQGLRAGMVAGVIVNRTQQEIP
NAETMKQTESHAVKIVVEAARRLL

In specific occurrences of this fifth instance, the at least one uridine phosphorylase enzyme is a uridine phosphorylase enzyme having the amino acid sequence as set forth below in SEQ ID NO: 28.

(SEQ ID NO: 28)
MHHHHHHGGSGMSKSDVFHLGLTKNDLQGATLAIVPGDPD
RVEKIAALMDKPVKLASHREFTTWRAELDGKPVIVCSTGI
GGPSTSIAVEILAQLGIRTFLRIGTTGAIQPHINVGDVLV
TTASVRLDGASLHFAPLEFPAVADFECTTALVEAAKSIGA
TTHVGVTASSDTFYPGQERYDTYSGRVVRHFKGSMEEWQA
MGVMNYEMESATLLTMCASQGLRAGMVAGVIVNRTQQEIP
NAETMKQTESHAVKIVVEAARRLL

In specific examples of such occurrences, the uridine phosphorylase enzyme comprising the amino acid sequence as set forth above in SEQ ID NO: 28 may be encoded by the DNA sequence as set forth below in SEQ ID NO: 29.

(SEQ ID NO: 29)
ATGCACCATCATCATCATCATGGCGGTAGCGGCATGTCCA
AGTCTGATGTTTTTCATCTCGGCCTCACTAAAAACGATTT
ACAAGGGGCTACGCTTGCCATCGTCCCTGGCGACCCGGAT
CGTGTGGAAAAGATCGCCGCGCTGATGGATAAGCCGGTTA
AGCTGGCATCTCACCGCGAATTCACTACCTGGCGTGCAGA
GCTGGATGGTAAACCTGTTATCGTCTGCTCTACCGGTATC
GGCGGCCCGTCTACCTCTATTGCTGTTGAAATTCTGGCAC
AGCTGGGCATTCGCACCTTCCTGCGTATCGGTACAACGGG
CGCTATTCAGCCGCATATTAATGTGGGTGATGTCCTGGTT
ACCACGGCGTCTGTCCGTCTGGATGGCGCGAGCCTGCACT
TCGCACCGCTGGAATTCCCGGCTGTCGCTGATTTCGAATG
TACGACTGCGCTGGTTGAAGCTGCGAAATCCATTGGCGCG
ACAACTCACGTTGGCGTGACAGCTTCTTCTGATACCTTCT
ACCCAGGTCAGGAACGTTACGATACTTACTCTGGTCGCGT
AGTTCGTCACTTTAAAGGTTCTATGGAAGAGTGGCAGGCG
ATGGGCGTAATGAACTATGAAATGGAATCTGCAACCCTGC
TGACCATGTGCGCAAGTCAGGGCCTGCGTGCCGGTATGGT
AGCGGGTGTTATCGTTAACCGCACCCAGCAAGAGATCCCG
AATGCTGAGACGATGAAACAAACCGAAAGCCATGCGGTGA
AAATCGTGGTGGAAGCGGCGCGTCGTCTGCTGTAA

In specific occurrences of this fifth instance, the at least one uridine phosphorylase enzyme is a uridine phosphorylase enzyme having the amino acid sequence as set forth below in SEQ ID NO: 30.

(SEQ ID NO: 30)
MHHHHHHGGSGMSESDSFHLGLTKNDLQGATLAILPGDPD
RVEKIAALMDKPVKLASWREFTTWRAELDGKPVIVCSTGI
GGPSTSIAVEILAQLGIRTFLRIGTTGAIQPHINVGDVLV
TTASVRLDGASLHFAPLEFPAVADFECTTALVEAAKSIGA
TTHVGVTASSDTFYPGQERYDTYSGRVVRHFKGSMEEWQR
MGVMNYEMESATLLTMCASQGLRAGMVAGVIVNRTQQELP
NAESMKQTESHMVKIVVEAARRLL

In specific occurrences of this fifth instance, the at least one uridine phosphorylase enzyme is a uridine phosphorylase enzyme having the amino acid sequence as set forth below in SEQ ID NO: 31.

(SEQ ID NO: 31)
MHHHHHHGGSGMSESDSFHMGLTKNDLQGATLAILPGDPA
RVEKIAALMDNPVKLASWREFTTWRAELDGKPVIVCSTGI
GGPSTSIAVEILAQLGIRTFLRIGTTGAIQGHINVGDVLV
TTASVRLDGASGHFAPMEFPAVADFECTTALVEAAKSIGA
TTHVGVTASSDTFYPGQERYDTYSGRVVRRFQGSMEEWQE
MGVMNYEMESATLLTMCASQGLRAGMVAGVIVNRTQQELP
NAESMKQVESHMVKIVVEAARRLL

In specific occurrences of this fifth instance, the at least one uridine phosphorylase enzyme is a uridine phosphorylase enzyme having the amino acid sequence as set forth below in SEQ ID NO: 45

(SEQ ID NO: 45)
MHHHHHHGGSGMSESDTFHMGLTKNDLQGATLAIVPGDPA
RVEKIAALMDNPVKLGSWREFITWRAELDGKPVIVCSTGI
GGPSTSIAVEILAQLGIRTFLRIGTTGAIQGHINVGDVLV
TTASVRLDGASGHFAPYEFPAVADFECTTALVEAAKSIGA
TTHVGVTASSDTFYPGQERYDTYSGRVVRRFQGSMEEWRE
MGVMNYEMESATLLTMCASQGLRAGMVAGVIVNRTQQELP
NAESMKQVESHMVKIVVEAARRLL

In specific occurrences of this fifth instance, the at least one uridine phosphorylase enzyme is a uridine phosphorylase enzyme having the amino acid sequence as set forth below in SEQ ID NO: 46

(SEQ ID NO: 46)
MHHHHHHGGSGMSESDTFHMGLTKNDLQGATLAILPGDPA
RVEKIAALMDNPVKLGSWREFITWRAELDGKPVIVCSTGI
GGPSTSIAVEILAQLGIRTFLRIGTTGAIQGHINVGDVLV
TTASVRLDGASGHFAPYEFPAVADFECTTALVEAAKSIGA
TTHVGVTASSDTFYPGQERYDTYSGRVVRRFQGSMEEWRV
MGVMNYEMESATLLTMCASQGLRAGMVAGVIVNRTQQELP
NAEDMPQVESHMVKIVVEAARRLL

In specific occurrences of this fifth instance, the at least one uridine phosphorylase enzyme is a uridine phosphorylase enzyme having the amino acid sequence as set forth below in SEQ ID NO: 47

(SEQ ID NO: 47)
MHHHHHHGGSGMSESDTFHMGLTKNDLQGATLAILPGDPA
RVEKIAALMDNPVKLGQWREFITWRAELDGKPVIVCSTGI
GGPSTSIAVEILAQLGIRTFLRIGTTGAIQGHINVGDVLV
TTASVRLDGASGHFAPYEFPAVADFECTTALVEAAKSCGA
TTHVGVTASSDTFYPGQERYDTYSGRVVRRFRGSMEEWRV
MGVMNYEMESATLLTMCASQGLRAGMVAGVIVNRTQQELP
NAESMPQVESFMVKIVVEAARRLL

In specific occurrences of this fifth instance, the at least one uridine phosphorylase enzyme is a uridine phosphorylase enzyme having the amino acid sequence as set forth below in SEQ ID NO: 48

(SEQ ID NO: 48)
MHHHHHHGGSGMSESDSFHMGLTKNDLQGATLAIVPGDPA
RVEKIAALMDNPVKLGQWREFITWRAELDGKPVIVCSTGI
GGPSTSIAVEILAQLGIRTFLRIGTTGAIQGHINVGDVLV
TTASVRLDGASGHFAPMEFPAVADFECTTALVEAAKSCGA
TTHVGVTASSDTFYPGQERYDTYSGRVVRRFQGSMEEWRE
MGVMNYEMESATLLTMCASQGLRAGMVAGVIVNRTQQELP
NAESMKQVESHMVKIVVEAARRLL

In specific occurrences of this fifth instance, the at least one uridine phosphorylase enzyme is a uridine phosphorylase enzyme having the amino acid sequence as set forth below in SEQ ID NO: 49

(SEQ ID NO: 49)
MHHHHHHGGSGMSESDSFHMGLTKNDLQGATLAIVPGDPA
RVEKIAALMDNPVKLGSWREFITWRAELDGKPVIVCSTGI
GGPSTSIAVEILAQLGIRTFLRIGTTGAIQGHIGVGDVLV
TTASVRLDGASGHFAPYEFPAVADFECTTALVEAAKSCGA
TTHVGVTASSDTFYPGQERYDTYSGRVVRRFRGSMEEWRE
MGVMNYEMESATLLTMCAVQGLRAGMVAGVIVNRTQQELP
NAEDMKQVESHMVKIVVEAARRLL

In specific occurrences of this fifth instance, the at least one uridine phosphorylase enzyme is a uridine phosphorylase enzyme having the amino acid sequence as set forth below in SEQ ID NO: 50

(SEQ ID NO: 50)
MHHHHHHGGSGMSESDSFHMGLTKNDLQGATLAILPGDPARVEKIAALM
DNPVKLGSWREFITWRAELDGKPVIVCSTGIGGPSTSIAVEILAQLGIR
TFLRIGTTGAIQGHIGVGDVLVTTASVRLDGASGHFAPYEFPAVADFEC
TTALVEAAKSIGATTHVGVTASSDTFYPGQERYDTYSGRVVRRFQGSME
EWREMGVMNYEMESATLLTMCASQGLRAGMVAGVIVNRTQQELPNAEDM
PQVESHMVKIVVEAARRLL

In specific occurrences of this fifth instance, the at least one uridine phosphorylase enzyme is a uridine phosphorylase enzyme having the amino acid sequence as set forth below in SEQ ID NO: 51

(SEQ ID NO: 51)
MHHHHHHGGSGMSESDSFHMGLTKNDLQGATLAIVPGDPARVEKIAALM
DNPVKLGSWREFITWRAELDGKPVIVCSTGIGGPSTSIAVEILAQLGIR
TFLRIGTTGAIQGHINVGDVLVTTASVRLDGASGHFAPMEFPAVADFEC
TTALVEAAKSIGATTHVGVTASSDTFYPGQERYDTYSGRVVRRFRGSME
EWREMGVMNYEMESATLLTMCASQGLRAGMVAGVIVNRTQQELPEAEDM
PQVESHMVKIVVEAARRLL

In some occurrences, the at least one uridine phosphorylase enzyme is a uridine phosphorylase enzyme based on the amino acid sequences of SEQ ID NO: 25, 27, 28, 30, 31, 45, 46, 47, 48, 49, 50, or 51 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 25, 27, 28, 30, 31, 45, 46, 47, 48, 49, 50, or 51. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one uridine phosphorylase enzyme is a uridine phosphorylase enzyme encoded by the DNA sequences of SEQ ID NO: 26 or 29 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 26 or 29. These differences can be amino acid insertions, deletions, substitutions, or any combinations of such changes. In some occurrences, the sequence differences can comprise non-conservative, conservative, as well as a combination of non-conservative and conservative amino acid substitutions.

In a third aspect of this second embodiment, the reacting is conducted in the presence of at least one solvent. In instances of this aspect, the at least one solvent is selected from aqueous solvents, organic solvents and mixtures thereof. In particular instances of this aspect, the at least one solvent is selected from the group consisting of water. In other particular instances of this aspect, the at least one solvent is selected from the group consisting of DME, anisole, tert-butanol, tert-amyl alcohol, acetone, 1,2-propylene carbonate, 1,3-dioxolane, acetonitrile, ethyl acetate, methyl tert-butyl ether, cyclopentyl methyl ether, chlorobenzene, isopropanol, isopropyl acetate, 2-butanone, and mixtures thereof. In still further instances of this aspect, the at least one solvent is water and at least one organic solvent selected from the group consisting of DME, anisole, tert-butanol, tert-amyl alcohol, acetone, 1,2-propylene carbonate, 1,3-dioxolane, acetonitrile, ethyl acetate, methyl tert-butyl ether, cyclopentyl methyl ether, chlorobenzene, isopropanol, isopropyl acetate, 2-butanone, and mixtures thereof.

In aspects of the second embodiment, the reacting comprises first reacting 5-isobutyrylribose with a phosphate source in the presence of at least one MTR kinase and at least one acetate kinase to produce a 5-isobutyrylribose hydrogen phosphate, and then reacting the 5-isobutyrylribose hydrogen phosphate with uracil in the presence of at least one uridine phosphorylase to form 5′-isobutyryluridine and hydrogen phosphate. In specific aspects, the hydrogen phosphate is reacted in the presence of at least one pyruvate oxidase and at least one catalase to regenerate the phosphate source.

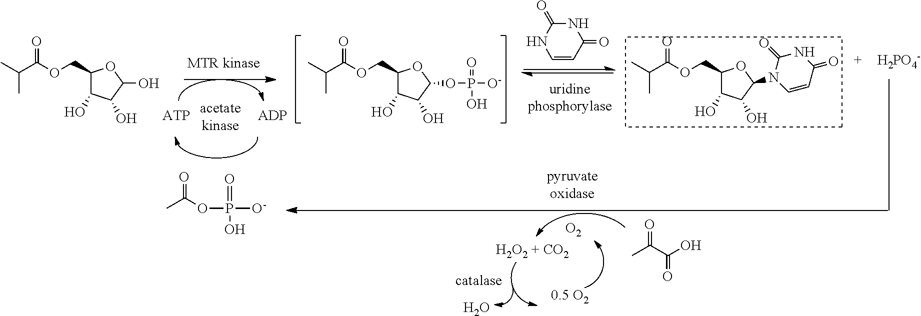

In a third embodiment of the processes of the disclosure, the process of the second embodiment further comprises reacting ribose with at least one isobutyryl donor in the presence of at least one lipase enzyme to form 5-isobutyrylribose:

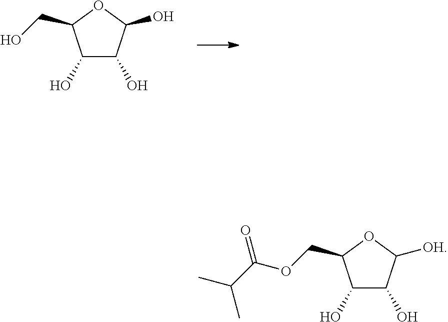

In a first aspect of this third embodiment, the at least one isobutyryl donor is selected from the group consisting of propan-2-one O-isobutyryl oxime, isobutyric anhydride, and mixtures thereof. In instances of this first aspect, the at least one isobutyryl donor is isobutyric anhydride. In specific instances of this aspect, the at least one isobutyryl donor is provided in an amount in a range of from about 1.0 to about 3.0 equivalents with respect to the amount of ribose, such as an amount of about 1.5 equivalents.

In a second aspect of this third embodiment, the at least one lipase enzyme is selected from the group consisting of wild-type lipase enzymes and lipase enzymes that are produced from the directed evolution from a commercially available, wild-type lipase enzyme. In instance of this aspect, the at least one lipase enzyme is selected from the group consisting of IMMTLL (commercially available as IMMTLL-T2-150, from ChiralVision), IMMRES (commercially available as IMMRES-T2-150, from ChiralVision), IMMLIPX (commercially available as IMMLIPX-T2-150, from ChiralVision), IMMP6-T2-250 (commercially available as IMMP6-T2-250, from ChiralVision), Novozym® 51032 (commercially available as catalog number 06-3135, from Strem Chemicals, Inc.), and Novozym® 435 (commercially available as catalog number 3925009-810, from Novozymes, or catalog number 06-3123, from Strem Chemicals, Inc.). In specific instances, the at least one lipase enzyme is Novozym® 435.

In some occurrences, the at least one lipase enzyme is a lipase enzyme that is at least about 85%, 86%, 87%, 88%, 89%, 90%, 91%, 92%, 93%, 94%, 95%, 96%, 97%, 98%, or 99% identical to the reference sequence of the commercially available, wild-type lipase enzyme. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one lipase enzyme is a lipase enzyme encoded by DNA sequences that are at least about 85%, 86%, 87%, 88%, 89%, 90%, 91%, 92%, 93%, 94%, 95%, 96%, 97%, 98%, or 99% identical to the reference sequence of the commercially available, wild-type lipase enzyme. These differences can be amino acid insertions, deletions, substitutions, or any combinations of such changes. In some occurrences, the sequence differences can comprise non-conservative, conservative, as well as a combination of non-conservative and conservative amino acid substitutions.

In a third aspect of this third embodiment, the reacting is conducted in the presence of at least one solvent. In instances of this aspect, the at least one solvent is selected from organic solvents and mixtures thereof. In particular instances of this aspect, the solvent is selected from the group consisting of DME, anisole, tert-butanol, tert-amyl alcohol, acetone, 1,2-propylene carbonate, 1,3-dioxolane, acetonitrile, ethyl acetate, methyl tert-butyl ether, cyclopentyl methyl ether, chlorobenzene, isopropanol, isopropyl acetate, 2-butanone, and mixtures thereof. In more particular instances, the at least one solvent is selected from the group consisting of tert-butanol, tert-amyl alcohol, acetone, and mixtures thereof. In still more particular instances, the at least one solvent is acetone.

A fourth embodiment of the processes of the disclosure comprises (a) reacting ribose with at least one isobutyryl donor in the presence of at least one lipase enzyme to form 5′-isobutyrylribose; (b) reacting 5′-isobutyrylribose with uracil in the presence of at least one enzyme, which is selected from the group consisting of S-methyl-5-thioribose kinase enzymes, acetate kinase enzymes, pyruvate oxidase enzymes, catalase enzymes; and uridine phosphorylase enzymes, and mixtures thereof, to form 5′-isobutyryluridine; and (c) reacting 5′-isobutyryluridine with at least one hydroxylamine source in the presence of at least one activating agent to produce Compound B:

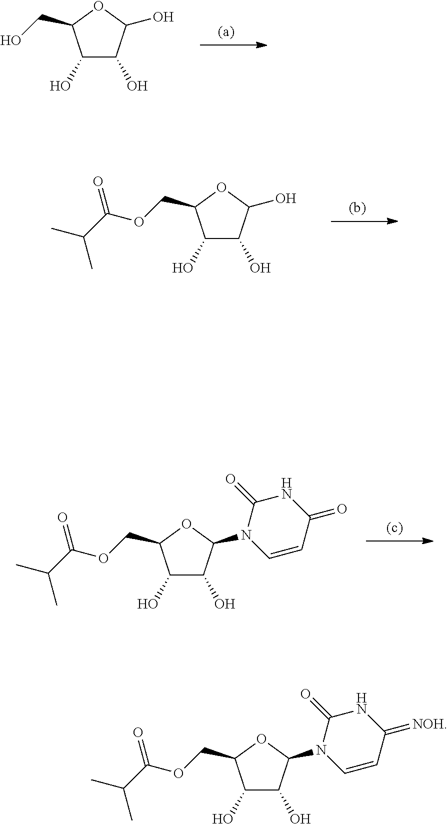

In a first aspect of the fourth embodiment, the at least one isobutyryl donor is selected from the group consisting of propan-2-one O-isobutyryl oxime, isobutyric anhydride, and mixtures thereof. In instances of this first aspect, the at least one isobutyryl donor is isobutyric anhydride. In specific instances of this aspect, the at least one isobutyryl donor is provided in an amount in a range of from about 1.0 to about 3.0 equivalents with respect to the amount of ribose, such as an amount of about 1.5 equivalents.

In a second aspect of this fourth embodiment, the at least one lipase enzyme is selected from the group consisting of wild-type lipase enzymes and lipase enzymes that are produced from the directed evolution from a commercially available, wild-type lipase enzyme. In instance of this aspect, the at least one lipase enzyme is selected from the group consisting of IMMTLL (commercially available as IMMTLL-T2-150, from ChiralVision), IMMRES (commercially available as IMMRES-T2-150, from ChiralVision), IMMLIPX (commercially available as IMMLIPX-T2-150, from ChiralVision), IMMP6-T2-250 (commercially available as IMMP6-T2-250, from ChiralVision), Novozym® 51032 (commercially available as catalog number 06-3135, from Strem Chemicals, Inc.), and Novozym® 435 (commercially available as catalog number 3925009-810, from Novozymes, or catalog number 06-3123, from Strem Chemicals, Inc.). In specific instances, the at least one lipase enzyme is Novozym® 435.

In some occurrences, the at least one lipase enzyme is a lipase enzyme that is at least about 85%, 86%, 87%, 88%, 89%, 90%, 91%, 92%, 93%, 94%, 95%, 96%, 97%, 98%, or 99% identical to the reference sequence of the commercially available, wild-type lipase enzyme. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one lipase enzyme is a lipase enzyme encoded by DNA sequences that are at least about 85%, 86%, 87%, 88%, 89%, 90%, 91%, 92%, 93%, 94%, 95%, 96%, 97%, 98%, or 99% identical to the reference sequence of the commercially available, wild-type lipase enzyme. These differences can be amino acid insertions, deletions, substitutions, or any combinations of such changes. In some occurrences, the sequence differences can comprise non-conservative, conservative, as well as a combination of non-conservative and conservative amino acid substitutions.

In a third aspect of this third embodiment, the (a) reacting is conducted in the presence of at least one solvent. In instances of this aspect, the at least one solvent is selected from organic solvents and mixtures thereof. In particular instances of this aspect, the solvent is selected from the group consisting of DME, anisole, tert-butanol, tert-amyl alcohol, acetone, 1,2-propylene carbonate, 1,3-dioxolane, acetonitrile, ethyl acetate, methyl tert-butyl ether, cyclopentyl methyl ether, chlorobenzene, isopropanol, isopropyl acetate, 2-butanone, and mixtures thereof. In more particular instances, the at least one solvent is selected from the group consisting of tert-butanol, tert-amyl alcohol, acetone, and mixtures thereof. In still more particular instances, the at least one solvent is acetone.

In a fourth aspect of this fourth embodiment, uracil is provided in an amount in a range of from about 0.5 to about 1.2 equivalents with respect to the amount of 5′-isobutyrylribose, such as an amount of about 0.8 equivalents.

In a fifth aspect of this fourth embodiment, the at least one enzyme is selected from the group consisting of at least one S-methyl-5-thioribose kinase enzyme, at least one acetate kinase enzyme, at least one pyruvate oxidase enzyme, at least one catalase enzyme; and at least one uridine phosphorylase enzyme, and mixtures thereof. That is, in instances of this fifth aspect, the at least one enzyme is selected from the group consisting of S-methyl-5-thioribose kinase enzymes, acetate kinase enzymes, pyruvate oxidase enzymes, catalase enzymes, and uridine phosphorylase enzymes, and mixtures thereof.

In a first instance of the fifth aspect of this fourth embodiment, the at least one S-methyl-5-thioribose kinase enzyme is one or more S-methyl-5-thioribose kinase selected from the group consisting of wild-type S-methyl-5-thioribose kinase enzymes and S-methyl-5-thioribose kinase enzymes that are produced from the directed evolution from a commercially available, wild-type S-methyl-5-thioribose kinase enzymes. In specific instances, the at least one S-methyl-5-thioribose kinase enzyme is selected from the group consisting of S-methyl-5-thioribose kinase enzymes that are produced from the directed evolution from a commercially available, wild-type S-methyl-5-thioribose kinase, which has the amino acid sequence as set forth below in SEQ ID NO: 1; in specific occurrences, the wild-type S-methyl-5-thioribose kinase comprising the amino acid sequence set forth above in SEQ ID NO: 1 may be encoded by the DNA sequence as set forth above in SEQ ID NO: 2. In specific occurrences of this first instance, the at least one enzyme is a S-methyl-5-thioribose kinase enzyme comprising the amino acid sequence as set forth above in SEQ ID NO: 3; in specific examples of such occurrences, the S-methyl-5-thioribose kinase comprising the amino acid sequence as set forth above in SEQ ID NO: 3 may be encoded by the DNA sequence as set forth above in SEQ ID NO: 4. In specific occurrences of this first instance, the at least one enzyme is a S-methyl-5-thioribose kinase enzyme comprising the amino acid sequence as set forth above in SEQ ID NO: 5; in specific examples of such occurrences, the S-methyl-5-thioribose kinase comprising the amino acid sequence as set forth above in SEQ ID NO: 5 may be encoded by the DNA sequence as set forth above in SEQ ID NO: 6. In specific occurrences of this instance, the at least one enzyme is a S-methyl-5-thioribose kinase enzyme comprising the amino acid sequence as set forth above in SEQ ID NO: 7; in specific examples of such occurrences, the S-methyl-5-thioribose kinase comprising the amino acid sequence as set forth above in SEQ ID NO: 7 may be encoded by the DNA sequence as set forth above in SEQ ID NO: 8. In specific occurrences of this instance, the at least one enzyme is a S-methyl-5-thioribose kinase enzyme comprising the amino acid sequence as set forth above in SEQ ID NO: 9; in specific examples of such occurrences, the S-methyl-5-thioribose kinase comprising the amino acid sequence as set forth above in SEQ ID NO: 9 may be encoded by the DNA sequence as set forth above in SEQ ID NO: 10. In specific occurrences of this instance, the at least one enzyme is a S-methyl-5-thioribose kinase enzyme comprising the amino acid sequence as set forth above in SEQ ID NO: 11; in specific examples of such occurrences, the S-methyl-5-thioribose kinase comprising the amino acid sequence as set forth above in SEQ ID NO: 11 may be encoded by the DNA sequence as set forth above in SEQ ID NO: 12. In specific occurrences of this instance, the at least one enzyme is a S-methyl-5-thioribose kinase enzyme comprising the amino acid sequence as set forth above in SEQ ID NO: 13.

In some occurrences, the at least one S-methyl-5-thioribose kinase enzyme is a S-methyl-5-thioribose kinase enzyme based on the amino acid sequences of SEQ ID NO: 1, 3, 5, 7, 9, 11, 12, or 13 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 1, 3, 5, 7, 9, 11, 12, or 13. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one S-methyl-5-thioribose kinase enzyme is a S-methyl-5-thioribose kinase enzyme encoded by the DNA sequences of SEQ ID NO: 2, 4, 6, 8, or 10 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 2, 4, 6, 8, or 10. These differences can be amino acid insertions, deletions, substitutions, or any combinations of such changes. In some occurrences, the sequence differences can comprise non-conservative, conservative, as well as a combination of non-conservative and conservative amino acid substitutions.

In a second instance of the fifth aspect of this fourth embodiment, the at least one acetate kinase enzyme is one or more acetate kinase selected from the group consisting of wild-type acetate kinase enzymes and acetate kinase enzymes that are produced from the directed evolution from a commercially available, wild-type acetate kinase enzymes. In specific instances, the at least one acetate kinase enzyme is selected from the group consisting of acetate enzymes that are produced from the directed evolution from a commercially available, wild-type acetate kinase enzyme, which has the amino acid sequence as set forth above in SEQ ID NO: 14. In specific occurrences, the wild-type acetate kinase enzyme comprising the amino acid sequence as set forth above in SEQ ID NO: 14 may be encoded by the DNA sequence as set forth above in SEQ ID NO: 15. In specific occurrences of this second instance, the at least one acetate kinase enzyme is an acetate kinase enzyme comprising the amino acid sequence as set forth above in SEQ ID NO: 16. In specific examples of such occurrences, the acetate kinase enzyme comprising the amino acid sequence as set forth above in SEQ ID NO: 16 may be encoded by the DNA sequence as set forth above in SEQ ID NO: 17.

In some occurrences of this second instance, the at least one acetate kinase enzyme is an acetate kinase enzyme based on the amino acid sequences of SEQ ID NO: 14 or 16 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 14 or 16. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences of this second instance, the at least one acetate kinase enzyme is an acetate kinase enzyme encoded by the DNA sequences of SEQ ID NO: 15 or 17 and can comprise an amino acid sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 15 or 17. These differences can be amino acid insertions, deletions, substitutions, or any combinations of such changes. In some occurrences, the sequence differences can comprise non-conservative, conservative, as well as a combination of non-conservative and conservative amino acid substitutions.

In a third instance of the fifth aspect of the fourth embodiment, the at least one pyruvate oxidase enzyme is selected from the group consisting of wild-type pyruvate oxidase enzymes and pyruvate oxidase enzymes that are produced from the directed evolution from a commercially available, wild-type pyruvate oxidase enzyme. In specific instances, the at least one pyruvate oxidase enzyme is selected from the group consisting of pyruvate oxidase enzymes that are produced from the directed evolution from a commercially available, wild-type pyruvate oxidase, which have the amino acid sequence as set forth above in SEQ ID NO: 18. In specific occurrences, the wild-type pyruvate oxidase enzyme having the amino acid sequence as set forth above in SEQ ID NO: 18 may be encoded by the DNA sequence as set forth above in SEQ ID NO: 19. In specific occurrences of this third instance, the at least one pyruvate oxidase enzyme is a pyruvate oxidase enzyme comprising the amino acid sequence as set forth above in SEQ ID NO: 20. In specific examples of such occurrences, the pyruvate oxidase enzyme comprising the amino acid sequence as set forth above in SEQ ID NO: 20 may be encoded by the DNA sequence as set forth above in SEQ ID NO: 21. In specific occurrences of this third instance, the at least one pyruvate oxidase enzyme is a pyruvate oxidase enzyme comprising the amino acid sequence as set forth above in SEQ ID NO: 22. In specific examples of such occurrences, the pyruvate oxidase enzyme comprising the amino acid sequence as set forth above in SEQ ID NO: 22 may be encoded by the DNA sequence as set forth above in SEQ ID NO: 23. In specific occurrences of this third instance, the at least one pyruvate oxidase enzyme is a pyruvate oxidase enzyme comprising the amino acid sequence as set forth above in SEQ ID NO: 24.

In some occurrences, the at least one pyruvate oxidase enzyme is a pyruvate oxidase enzyme based on the amino acid sequences of SEQ ID NO: 18, 20, 22, or 24 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 18, 20, 22, or 24. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one pyruvate oxidase enzyme is a pyruvate oxidase enzyme encoded by the DNA sequences of SEQ ID NO: 19, 21, or 23 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 19, 21, or 23. These differences can be amino acid insertions, deletions, substitutions, or any combinations of such changes. In some occurrences, the sequence differences can comprise non-conservative, conservative, as well as a combination of non-conservative and conservative amino acid substitutions.

In a fourth instance of the fifth aspect of the fourth embodiment, the at least one catalase enzyme is selected from the group consisting of wild-type catalase enzymes and catalase enzymes that are produced from the directed evolution from a commercially available, wild-type catalase, including the catalase enzyme commercially available as product number 11650645103 from Roche Diagnostics International Ltd. In specific occurrences of this fourth instance, the at least one catalase enzyme is the wild-type catalase enzyme that comprises the amino acid sequence as set forth above in SEQ ID NO: 52. In specific examples of such occurrences, the catalase enzyme comprising the amino acid sequence as set forth above in SEQ ID NO: 52 may be encoded by the DNA sequence as set forth above in SEQ ID NO: 53. In specific examples of such occurrences, the catalase enzyme comprising the amino acid sequence as set forth above in SEQ ID NO: 52 may be encoded by the DNA sequence as set forth above in SEQ ID NO: 54.

In some occurrences, the at least one catalase enzyme is at least about 85%, 86%, 87%, 88%, 89%, 90%, 91%, 92%, 93%, 94%, 95%, 96%, 97%, 98%, or 99% identical to the reference sequence of the catalase enzyme commercially available as product number 11650645103 from Roche Diagnostics International Ltd. In some occurrences, the at least one catalase enzyme is at least about 85%, 86%, 87%, 88%, 89%, 90%, 91%, 92%, 93%, 94%, 95%, 96%, 97%, 98%, or 99% identical to the reference sequence SEQ ID NO: 52. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one catalase enzyme is encoded by DNA sequences that are at least about 85%, 86%, 87%, 88%, 89%, 90%, 91%, 92%, 93%, 94%, 95%, 96%, 97%, 98%, or 99% identical to the reference sequence of the catalase enzyme commercially available as product number 11650645103 from Roche Diagnostics International Ltd. In some occurrences, the at least one catalase enzyme is encoded by DNA sequences that are at least about 85%, 86%, 87%, 88%, 89%, 90%, 91%, 92%, 93%, 94%, 95%, 96%, 97%, 98%, or 99% identical to SEQ ID NO: 53 or 54. These differences can be amino acid insertions, deletions, substitutions, or any combinations of such changes. In some occurrences, the sequence differences can comprise non-conservative, conservative, as well as a combination of non-conservative and conservative amino acid substitutions.

In a fifth instance of the fifth aspect of this fourth embodiment, the at least one uridine phosphorylase enzyme is selected from the group consisting of wild-type uridine phosphorylase enzymes and uridine phosphorylase enzymes that are produced from the directed evolution from a commercially available, wild-type uridine phosphorylase enzyme. In specific instances, the at least one uridine phosphorylase enzyme is a wild-type uridine phosphorylase enzyme having the amino acid sequence as set forth above in SEQ ID NO: 25. In specific occurrences, the wild-type uridine phosphorylase enzyme comprising the amino acid sequence as set forth above in SEQ ID NO: 25 may be encoded by the DNA sequence as set forth above in SEQ ID NO: 26. In specific occurrences of this fifth instance, the at least one uridine phosphorylase enzyme is a uridine phosphorylase enzyme having the amino acid sequence as set forth above in SEQ ID NO: 27. In specific occurrences of this fifth instance, the at least one uridine phosphorylase enzyme is a uridine phosphorylase enzyme having the amino acid sequence as set forth above in SEQ ID NO: 28. In specific examples of such occurrences, the uridine phosphorylase enzyme comprising the amino acid sequence as set forth above in SEQ ID NO: 28 may be encoded by the DNA sequence as set forth above in SEQ ID NO: 29. In specific instances, the at least one uridine phosphorylase enzyme is a uridine phosphorylase having the amino acid sequence as set forth above in SEQ ID NO: 30. In specific instances, the at least one uridine phosphorylase enzyme is a uridine phosphorylase having the amino acid sequence as set forth above in SEQ ID NO: 31. In specific instances, the at least one uridine phosphorylase enzyme is a uridine phosphorylase having the amino acid sequence as set forth above in SEQ ID NO: 45. In specific instances, the at least one uridine phosphorylase enzyme is a uridine phosphorylase having the amino acid sequence as set forth above in SEQ ID NO: 46. In specific instances, the at least one uridine phosphorylase enzyme is a uridine phosphorylase having the amino acid sequence as set forth above in SEQ ID NO: 47. In specific instances, the at least one uridine phosphorylase enzyme is a uridine phosphorylase having the amino acid sequence as set forth above in SEQ ID NO: 48. In specific instances, the at least one uridine phosphorylase enzyme is a uridine phosphorylase having the amino acid sequence as set forth above in SEQ ID NO: 49. In specific instances, the at least one uridine phosphorylase enzyme is a uridine phosphorylase having the amino acid sequence as set forth above in SEQ ID NO: 50. In specific instances, the at least one uridine phosphorylase enzyme is a uridine phosphorylase having the amino acid sequence as set forth above in SEQ ID NO: 51.

In some occurrences, the at least one uridine phosphorylase enzyme is a uridine phosphorylase enzyme based on the amino acid sequences of SEQ ID NO: 25, 27, 28, 30, 31, 45, 46, 47, 48, 49, 50, or 51 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 25, 27, 28, 30, 31, 45, 46, 47, 48, 49, 50, or 51. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one uridine phosphorylase enzyme is a uridine phosphorylase enzyme encoded by the DNA sequences of SEQ ID NO: 26 or 29 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 26 or 29. These differences can be amino acid insertions, deletions, substitutions, or any combinations of such changes. In some occurrences, the sequence differences can comprise non-conservative, conservative, as well as a combination of non-conservative and conservative amino acid substitutions.

In a sixth aspect of this fourth embodiment, the (b) reacting is conducted in the presence of at least one solvent. In instances of this aspect, the at least one solvent is selected from aqueous solvents, organic solvents and mixtures thereof. In particular instances of this aspect, the at least one solvent is selected from the group consisting of water. In other particular instances of this aspect, the at least one solvent is selected from the group consisting of DME, anisole, tert-butanol, tert-amyl alcohol, acetone, 1,2-propylene carbonate, 1,3-dioxolane, acetonitrile, ethyl acetate, methyl tert-butyl ether, cyclopentyl methyl ether, chlorobenzene, isopropanol, isopropyl acetate, 2-butanone, and mixtures thereof. In still further instances of this aspect, the at least one solvent is water and at least one organic solvent selected from the group consisting of DME, anisole, tert-butanol, tert-amyl alcohol, acetone, 1,2-propylene carbonate, 1,3-dioxolane, acetonitrile, ethyl acetate, methyl tert-butyl ether, cyclopentyl methyl ether, chlorobenzene, isopropanol, isopropyl acetate, 2-butanone, and mixtures thereof.

In a seventh aspect of this fourth embodiment, the at least one hydroxylamine source is selected from the group consisting of hydroxylamine sulfate, hydroxylamine HCl, NH₂OH, and mixtures thereof. In instances of this seventh aspect, the at least one hydroxylamine source is hydroxylamine sulfate. In specific instances of this aspect, the at least one hydroxylamine source is provided in an amount to provide hydroxylamine in a range of from about 1.0 to about 2.5 equivalents of hydroxylamine with respect to the amount of 5′-isobutyryluridine, such as an amount in a range of from about 2.2 to about 2.5 equivalents, or an amount of about 2.5 equivalents.

In an eighth aspect of this fourth embodiment, the at least one activating agent is hexamethyldisilazane. In specific instances of this aspect, the at least one activating agent is provided in an amount in a range of from about 6.0 to about 8.0 equivalents with respect to the amount of 5′-isobutyryluridine, such as an amount of about 8 equivalents.

In a ninth aspect of this fourth embodiment, the (c) reacting is conducted in the presence of at least one acidic additive. In instances of this ninth aspect, the at least one acidic additive is selected from the group consisting of ammonium bisulfate, ammonium dihydrogen phosphate, sulfuric acid, sodium bisulfate, potassium bisulfate, imidazole bisulfate, triethylamine bisulfate, N-methylmorpholine bisulfate, N-methylimidazole bisulfate, sulfur trioxide pyridine complex, trifluoromethanesulfonic acid, methanesulfonic acid, and mixtures thereof. In specific instances, the at least one acidic additive is ammonium bisulfate. In specific instances, the at least one acidic additive is present in an amount in a range of from about 1.5 to about 3.0 equivalents with respect to the amount of 5′-isobutyryluridine.

In a tenth aspect of this fourth embodiment, the (c) reacting is optionally conducted in the presence of at least one catalyst. In certain instances of this aspect, the at least one catalyst is selected from Lewis basic catalysts. In instances of this tenth aspect, the at least one catalyst is selected from the group consisting of N-methylimidazole, N-methylmorpholine, 1,2,4-triazole, 5-(ethylthio)-1H-tetrazole, imidazole, and mixtures thereof. In specific instances, the at least one catalyst is imidazole. In specific instances, the at least one catalyst is present in an amount in a range of from about 0.2 to about 0.5 equivalents with respect to the amount of 5′-isobutyryluridine.

In an eleventh aspect of this fourth embodiment, the (c) reacting is conducted in the presence of at least one solvent. In instances of this aspect, the at least one solvent is selected from organic solvents and mixtures thereof. In particular instances of this aspect, the at least one solvent is selected from the group consisting of heptane, toluene, 2-methyltetrahydrofuran, tetrahydrofuran, DME, sulfolane, and mixtures thereof.

A fifth embodiment of the processes of the disclosure, the process of the first embodiment comprises reacting uridine with at least one isobutyryl donor in the presence of at least one lipase enzyme to form 5′-isobutyryluridine:

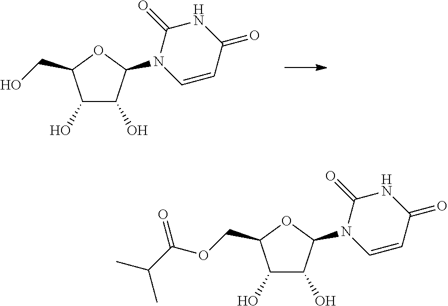 [SMILES: O=c1ccn([C@@H]2O[C@H](CO)[C@@H](O)[C@H]2O)c(=O)[nH]1 ; CC(C)C(=O)OC[C@H]1O[C@@H](n2ccc(=O)[nH]c2=O)[C@H](O)[C@@H]1O]

In a first aspect of this fifth embodiment, the at least one isobutyryl donor is selected from the group consisting of propan-2-one O-isobutyryl oxime, isobutyric anhydride, and mixtures thereof. In instances of this first aspect, the at least one isobutyryl donor is isobutyric anhydride. In specific instances of this aspect, the at least one isobutyryl donor is provided in an amount in a range of from about 1.0 to about 3.0 equivalents with respect to the amount of ribose, such as an amount of about 1.5 equivalents.

In a second aspect of this fifth embodiment, the at least one lipase enzyme is selected from the group consisting of wild-type lipase enzymes and lipase enzymes that are produced from the directed evolution from a commercially available, wild-type lipase enzyme. In instance of this aspect, the at least one lipase enzyme is selected from the group consisting of IMMTLL (commercially available as IMMTLL-T2-150, from ChiralVision), IMMRES (commercially available as IMMRES-T2-150, from ChiralVision), IMMLIPX (commercially available as IMMLIPX-T2-150, from ChiralVision), IMMP6-T2-250 (commercially available as IMMP6-T2-250, from ChiralVision), Novozym® 51032 (commercially available as catalog number 06-3135, from Strem Chemicals, Inc.), and Novozym® 435 (commercially available as catalog number 3925009-810, from Novozymes, or catalog number 06-3123, from Strem Chemicals, Inc.). In specific instances, the at least one lipase enzyme is Novozym® 435.

In some occurrences, the at least one lipase enzyme is a lipase enzyme that is at least about 85%, 86%, 87%, 88%, 89%, 90%, 91%, 92%, 93%, 94%, 95%, 96%, 97%, 98%, or 99% identical to the reference sequence of the commercially available, wild-type lipase enzyme. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one lipase enzyme is a lipase enzyme encoded by DNA sequences that are at least about 85%, 86%, 87%, 88%, 89%, 90%, 91%, 92%, 93%, 94%, 95%, 96%, 97%, 98%, or 99% identical to the reference sequence of the commercially available, wild-type lipase enzyme. These differences can be amino acid insertions, deletions, substitutions, or any combinations of such changes. In some occurrences, the sequence differences can comprise non-conservative, conservative, as well as a combination of non-conservative and conservative amino acid substitutions.

In a third aspect of this fifth embodiment, the (a) reacting is conducted in the presence of at least one solvent. In instances of this aspect, the at least one solvent is selected from organic solvents and mixtures thereof. In particular instances of this aspect, the solvent is selected from the group consisting of DME, anisole, tert-butanol, tert-amyl alcohol, acetone, 1,2-propylene carbonate, 1,3-dioxolane, acetonitrile, ethyl acetate, methyl tert-butyl ether, cyclopentyl methyl ether, chlorobenzene, isopropanol, isopropyl acetate, 2-butanone, and mixtures thereof. In more particular instances, the at least one solvent is selected from the group consisting of tert-butanol, tert-amyl alcohol, acetone, and mixtures thereof. In still more particular instances, the at least one solvent is acetone.

A sixth embodiment of the processes of the disclosure, the process of the fifth embodiment further comprises reacting ribose with uracil in the presence of at least one enzyme to form uridine:

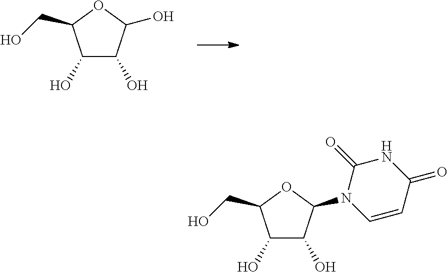 [SMILES: OC[C@H]1OC(O)[C@H](O)[C@@H]1O ; O=c1ccn([C@@H]2O[C@H](CO)[C@@H](O)[C@H]2O)c(=O)[nH]1]

In a first aspect of this sixth embodiment, uracil is provided in an amount in a range of from about 0.4 to about 1.2 equivalents with respect to the amount of ribose, such as an amount of about 0.8 equivalents.

In a second aspect of this sixth embodiment, the at least one enzyme is selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme (alternatively referred to as “ribokinase enzymes”), at least one phosphopentomutase enzyme (alternatively referred to as “phosphopentomutase enzymes”), and at least one sucrose phosphorylase enzyme (alternatively referred to as “sucrose phosphorylase enzymes”), and mixtures thereof.

In instances of this second aspect, the at least one enzyme is a mixture of at least two enzymes selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme, at least one phosphopentomutase enzyme, and at least one sucrose phosphorylase enzyme. In some instances, the at least one enzyme is a mixture of at least three enzymes selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme, at least one phosphopentomutase enzyme, and at least one sucrose phosphorylase enzyme. In further instances, the at least one enzyme is a mixture of at least four enzymes selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme, at least one phosphopentomutase enzyme, and at least one sucrose phosphorylase enzyme. In still further instances, the at least one enzyme is a mixture of at least five enzymes selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme, at least one phosphopentomutase enzyme, and at least one sucrose phosphorylase enzyme. In particular instances, the at least one enzyme is a mixture of at least one S-methyl-5-thioribose kinase enzyme, at least one acetate kinase enzyme, at least one pyruvate oxidase enzyme, at least one catalase enzyme, and at least one uridine phosphorylase enzyme. In other particular instances, the at least one enzyme is a mixture of at least one S-methyl-5-thioribose kinase enzyme, at least one acetate kinase enzyme, at least one uridine phosphorylase enzyme, and at least one sucrose phosphorylase enzyme. In additional particular instances, the at least one enzyme is a mixture of at least one acetate kinase enzyme, at least one pyruvate oxidase enzyme, at least one catalase enzyme, at least one uridine phosphorylase enzyme, at least one ribokinase enzyme, and at least one phosphopentomutase enzyme. In further particular instances, the at least one enzyme is a mixture of at least one S-methyl-5-thioribose kinase enzyme, at least one acetate kinase enzyme, at least one uridine phosphorylase enzyme, at least one ribokinase enzyme, at least one phosphopentomutase enzyme, and at least one sucrose phosphorylase enzyme. In still further particular instances, the at least one enzyme is a mixture of at least one acetate kinase enzyme, at least one uridine phosphorylase enzyme, and at least two enzymes selected from the group consisting of at least one S-methyl-5-thioribose kinase enzyme, at least one pyruvate oxidase enzyme, at least one catalase enzyme, at least one ribokinase enzyme, at least one phosphopentomutase enzyme, and at least one sucrose phosphorylase enzyme.

In a first instance of the second aspect of this sixth embodiment, the at least one S-methyl-5-thioribose kinase enzyme is one or more S-methyl-5-thioribose kinase enzymes selected from the group consisting of wild-type S-methyl-5-thioribose kinase enzymes and S-methyl-5-thioribose kinase enzymes that are produced from the directed evolution from a commercially available, wild-type S-methyl-5-thioribose kinase enzyme. In specific instances, the at least one S-methyl-5-thioribose kinase enzyme is selected from the group consisting of S-methyl-5-thioribose kinase enzymes that are produced from the directed evolution from a commercially available, wild-type S-methyl-5-thioribose kinase enzyme, which has the amino acid sequence as set forth above in SEQ ID NO: 1; in specific occurrences, the wild-type S-methyl-5-thioribose kinase enzyme comprising the amino acid sequence set forth above in SEQ ID NO: 1 may be encoded by the DNA sequence as set forth above in SEQ ID NO: 2. In specific occurrences of this first instance, the at least one S-methyl-5-thioribose kinase enzyme is a S-methyl-5-thioribose kinase enzyme comprising the amino acid sequence as set forth above in SEQ ID NO: 3. In specific examples of such occurrences, the S-methyl-5-thioribose kinase enzyme comprising the amino acid sequence as set forth above in SEQ ID NO: 3 may be encoded by the DNA sequence as set forth above in SEQ ID NO: 4. In specific occurrences of this first instance, the at least one S-methyl-5-thioribose kinase enzyme is a S-methyl-5-thioribose kinase enzyme comprising the amino acid sequence as set forth above in SEQ ID NO: 5. In specific examples of such occurrences, the S-methyl-5-thioribose kinase enzyme comprising the amino acid sequence as set forth above in SEQ ID NO: 5 may be encoded by the DNA sequence as set forth above in SEQ ID NO: 6. In specific occurrences of this instance, the at least one S-methyl-5-thioribose kinase enzyme is a S-methyl-5-thioribose kinase enzyme comprising the amino acid sequence as set forth above in SEQ ID NO: 7. In specific examples of such occurrences, the S-methyl-5-thioribose kinase enzyme comprising the amino acid sequence as set forth above in SEQ ID NO: 7 may be encoded by the DNA sequence as set forth above in SEQ ID NO: 8. In specific occurrences of this instance, the at least one S-methyl-5-thioribose kinase enzyme is a S-methyl-5-thioribose kinase enzyme comprising the amino acid sequence as set forth above in SEQ ID NO: 9. In specific examples of such occurrences, the S-methyl-5-thioribose kinase enzyme comprising the amino acid sequence as set forth above in SEQ ID NO: 9 may be encoded by the DNA sequence as set forth above in SEQ ID NO: 10. In specific occurrences of this instance, the at least one S-methyl-5-thioribose kinase enzyme is a S-methyl-5-thioribose kinase enzyme comprising the amino acid sequence as set forth above in SEQ ID NO: 11. In specific examples of such occurrences, the S-methyl-5-thioribose kinase enzyme comprising the amino acid sequence as set forth above in SEQ ID NO: 11 may be encoded by the DNA sequence as set forth above in SEQ ID NO: 12. In specific occurrences of this instance, the at least one S-methyl-5-thioribose kinase enzyme is a S-methyl-5-thioribose kinase enzyme comprising the amino acid sequence as set forth above in SEQ ID NO: 13.

In some occurrences, the at least one enzyme is a S-methyl-5-thioribose kinase enzyme based on the amino acid sequences of SEQ ID NO: 1, 3, 5, 7, 9, 11, 12, or 13 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 1, 3, 5, 7, 9, 11, 12, or 13. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one enzyme is a S-methyl-5-thioribose kinase enzyme encoded by the DNA sequences of SEQ ID NO: 2, 4, 6, 8, or 10 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 2, 4, 6, 8, or 10. These differences can be amino acid insertions, deletions, substitutions, or any combinations of such changes. In some occurrences, the sequence differences can comprise non-conservative, conservative, as well as a combination of non-conservative and conservative amino acid substitutions.

In a second instance of the second aspect of this sixth embodiment, the at least one acetate kinase enzyme is one or more acetate kinase enzymes selected from the group consisting of wild-type acetate kinase enzymes and acetate kinase enzymes that are produced from the directed evolution from a commercially available, wild-type acetate kinase enzyme. In specific instances, the at least one acetate kinase enzyme is selected from the group consisting of acetate kinase enzymes that are produced from the directed evolution from a commercially available, wild-type acetate kinase enzyme, which has the amino acid sequence as set forth above in SEQ ID NO: 14. In specific occurrences, the wild-type acetate kinase enzyme comprising the amino acid sequence as set forth above in SEQ ID NO: 14 may be encoded by the DNA sequence as set forth above in SEQ ID NO: 15. In specific occurrences of this second instance, the at least one acetate kinase enzyme is an acetate kinase enzyme comprising the amino acid sequence as set forth above in SEQ ID NO: 16. In specific examples of such occurrences, the acetate kinase enzyme comprising the amino acid sequence as set forth above in SEQ ID NO: 16 may be encoded by the DNA sequence as set forth above in SEQ ID NO: 17.

In some occurrences of this second instance, the at least one acetate kinase enzyme is an acetate kinase enzyme based on the amino acid sequences of SEQ ID NO: 14 or 16 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 14 or 16. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences of this second instance, the at least one acetate kinase enzyme is an acetate kinase enzyme encoded by the DNA sequences of SEQ ID NO: 15 or 17 and can comprise an amino acid sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 15 or 17. These differences can be amino acid insertions, deletions, substitutions, or any combinations of such changes. In some occurrences, the sequence differences can comprise non-conservative, conservative, as well as a combination of non-conservative and conservative amino acid substitutions.

In a third instance of the second aspect of this sixth embodiment, the at least one pyruvate oxidase enzyme is one or more pyruvate oxidase enzymes selected from the group consisting of wild-type pyruvate oxidase enzymes and pyruvate oxidase enzymes that are produced from the directed evolution from a commercially available, wild-type pyruvate oxidase enzyme. In specific instances, the at least one pyruvate oxidase enzyme is selected from the group consisting of pyruvate oxidase enzymes that are produced from the directed evolution from a commercially available, wild-type pyruvate oxidase enzyme, which has the amino acid sequence as set forth above in SEQ ID NO: 18. In specific occurrences, the wild-type pyruvate oxidase enzyme having the amino acid sequence as set forth above in SEQ ID NO: 18 may be encoded by the DNA sequence as set forth above in SEQ ID NO: 19. In specific occurrences of this third instance, the at least one pyruvate oxidase enzyme is a pyruvate oxidase enzyme comprising the amino acid sequence as set forth above in SEQ ID NO: 20. In specific examples of such occurrences, the pyruvate oxidase enzyme comprising the amino acid sequence as set forth above in SEQ ID NO: 20 may be encoded by the DNA sequence as set forth above in SEQ ID NO: 21. In specific occurrences of this third instance, the at least one pyruvate oxidase enzyme is a pyruvate oxidase enzyme comprising the amino acid sequence as set forth above in SEQ ID NO: 22. In specific examples of such occurrences, the pyruvate oxidase enzyme comprising the amino acid sequence as set forth above in SEQ ID NO: 22 may be encoded by the DNA sequence as set forth above in SEQ ID NO: 23. In specific occurrences of this third instance, the at least one pyruvate oxidase enzyme is a pyruvate oxidase enzyme comprising the amino acid sequence as set forth above in SEQ ID NO: 24.

In some occurrences, the at least one pyruvate oxidase enzyme is a pyruvate oxidase enzyme based on the amino acid sequences of SEQ ID NO: 18, 20, 22, or 24 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 18, 20, 22, or 24. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one pyruvate oxidase enzyme is a pyruvate oxidase enzyme encoded by the DNA sequences of SEQ ID NO: 19, 21, or 23 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 19, 21, or 23. These differences can be amino acid insertions, deletions, substitutions, or any combinations of such changes. In some occurrences, the sequence differences can comprise non-conservative, conservative, as well as a combination of non-conservative and conservative amino acid substitutions.

In a fourth instance of the second aspect of this sixth embodiment, the at least one catalase enzyme is one or more catalase enzymes selected from the group consisting of wild-type catalase enzymes and catalase enzymes that are produced from the directed evolution from a commercially available, wild-type catalase enzyme. In specific instances, the at least one catalase enzyme is selected from the group consisting of catalase enzymes that are produced from the directed evolution from a commercially available, wild-type catalase enzyme, including the catalase enzyme commercially available as product number 11650645103 from Roche Diagnostics International Ltd. In specific occurrences of this fourth instance, the at least one catalase enzyme is the wild-type catalase enzyme that comprises the amino acid sequence as set forth above in SEQ ID NO: 52. In specific examples of such occurrences, the catalase enzyme comprising the amino acid sequence as set forth above in SEQ ID NO: 52 may be encoded by the DNA sequence as set forth above in SEQ ID NO: 53. In specific examples of such occurrences, the catalase enzyme comprising the amino acid sequence as set forth above in SEQ ID NO: 52 may be encoded by the DNA sequence as set forth above in SEQ ID NO: 54.

In some occurrences, the at least one catalase enzyme is at least about 85%, 86%, 87%, 88%, 89%, 90%, 91%, 92%, 93%, 94%, 95%, 96%, 97%, 98%, or 99% identical to the reference sequence of the catalase enzyme commercially available as product number 11650645103 from Roche Diagnostics International Ltd. In some occurrences, the at least one catalase enzyme is at least about 85%, 86%, 87%, 88%, 89%, 90%, 91%, 92%, 93%, 94%, 95%, 96%, 97%, 98%, or 99% identical to the reference sequence SEQ ID NO: 52. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one catalase enzyme is encoded by DNA sequences that are at least about 85%, 86%, 87%, 88%, 89%, 90%, 91%, 92%, 93%, 94%, 95%, 96%, 97%, 98%, or 99% identical to the reference sequence of the catalase enzyme commercially available as product number 11650645103 from Roche Diagnostics International Ltd. In some occurrences, the at least one catalase enzyme is encoded by DNA sequences that are at least about 85%, 86%, 87%, 88%, 89%, 90%, 91%, 92%, 93%, 94%, 95%, 96%, 97%, 98%, or 99% identical to SEQ ID NO: 53 or 54. These differences can be amino acid insertions, deletions, substitutions, or any combinations of such changes. In some occurrences, the sequence differences can comprise non-conservative, conservative, as well as a combination of non-conservative and conservative amino acid substitutions.

In a fifth instance of the second aspect of this sixth embodiment, the at least one uridine phosphorylase enzyme is one or more uridine phosphorylase enzymes selected from the group consisting of wild-type uridine phosphorylase enzymes and uridine phosphorylase enzymes that are produced from the directed evolution from a commercially available, wild-type uridine phosphorylase enzyme. In specific instances, the at least one uridine phosphorylase enzyme is selected from the group consisting of uridine phosphorylase enzymes that are produced from the directed evolution from a commercially available, wild-type uridine phosphorylase enzyme, which has the amino acid sequence as set forth above in SEQ ID NO: 25. In specific occurrences, the wild-type uridine phosphorylase enzyme comprising the amino acid sequence as set forth above in SEQ ID NO: 25 may be encoded by the DNA sequence as set forth above in SEQ ID NO: 26. In specific occurrences of this fifth instance, the at least one uridine phosphorylase enzyme is selected from a uridine phosphorylase enzyme having the amino acid sequence as set forth above in SEQ ID NO: 27. In specific occurrences of this fifth instance, the at least one uridine phosphorylase enzyme is selected from a uridine phosphorylase enzyme having the amino acid sequence as set forth above in SEQ ID NO: 28. In specific examples of such occurrences, the uridine phosphorylase enzyme comprising the amino acid sequence as set forth above in SEQ ID NO: 28 may be encoded by the DNA sequence as set forth above in SEQ ID NO: 29. In specific instances, the at least one uridine phosphorylase enzyme is selected from a uridine phosphorylase having the amino acid sequence as set forth above in SEQ ID NO: 30. In specific instances, the at least one uridine phosphorylase enzyme is selected from a uridine phosphorylase having the amino acid sequence as set forth above in SEQ ID NO: 31. In specific instances, the at least one uridine phosphorylase enzyme is a uridine phosphorylase having the amino acid sequence as set forth above in SEQ ID NO: 45. In specific instances, the at least one uridine phosphorylase enzyme is a uridine phosphorylase having the amino acid sequence as set forth above in SEQ ID NO: 46. In specific instances, the at least one uridine phosphorylase enzyme is a uridine phosphorylase having the amino acid sequence as set forth above in SEQ ID NO: 47. In specific instances, the at least one uridine phosphorylase enzyme is a uridine phosphorylase having the amino acid sequence as set forth above in SEQ ID NO: 48. In specific instances, the at least one uridine phosphorylase enzyme is a uridine phosphorylase having the amino acid sequence as set forth above in SEQ ID NO: 49. In specific instances, the at least one uridine phosphorylase enzyme is a uridine phosphorylase having the amino acid sequence as set forth above in SEQ ID NO: 50. In specific instances, the at least one uridine phosphorylase enzyme is a uridine phosphorylase having the amino acid sequence as set forth above in SEQ ID NO: 51.

In some occurrences, the at least one uridine phosphorylase enzyme is a uridine phosphorylase enzyme based on the amino acid sequences of SEQ ID NO: 25, 27, 28, 30, 31, 45, 46, 47, 48, 49, 50, or 51 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 25, 27, 28, 30, 31, 45, 46, 47, 48, 49, 50, or 51. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one uridine phosphorylase enzyme is a uridine phosphorylase enzyme encoded by the DNA sequences of SEQ ID NO: 26 or 29 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 26 or 29. These differences can be amino acid insertions, deletions, substitutions, or any combinations of such changes. In some occurrences, the sequence differences can comprise non-conservative, conservative, as well as a combination of non-conservative and conservative amino acid substitutions.

In a sixth instance of the second aspect, the at least one ribokinase enzyme is one or more ribokinase enzymes selected from the group consisting of wild-type ribokinase enzymes and ribokinase enzymes that are produced from the directed evolution from a commercially available, wild-type ribokinase enzyme. In specific occurrences, the at least one ribokinase enzyme is a wild-type ribokinase enzyme that comprises the amino acid sequence as set forth below in SEQ ID NO: 32.

(SEQ ID NO: 32)
MQNAGSLVVLGSINADHILNLQSFPTPGETVTGNHYQVAFGGKGANQAV
AAGRSGANIAFIACTGDDSIGESVRQQLATDNIDITPVSVIKGESTGVA
LIFVNGEGENVIGIHAGANAALSPALVEAQRERIANASALLMQLESPLE
SVMAAAKIAHQNKTIVALNPAPARELPDELLALVDIITPNETEAEKLTG
IRVENDEDAAKAAQVLHEKGIRTVLITLGSRGVWASVNGEGQRVPGFRV
QAVDTIAAGDTFNGALITALLEEKPLPEAIRFAHAAAAIAVTRKGAQPS
VPWREEIDAFLDRQR

In specific occurrences, the wild-type ribokinase enzyme comprising the amino acid sequence set forth above as SEQ ID NO: 32 may be encoded by the DNA sequence as set forth below in SEQ ID NO: 33.

(SEQ ID NO: 33)
ATGCAAAACGCAGGCAGCCTCGTTGTTCTTGGCAGCATTAATGCTGACC
ACATTCTTAATCTTCAATCTTTTCCTACTCCAGGCGAAACCGTAACCGG
TAACCACTATCAGGTTGCATTTGGCGGCAAAGGCGCGAATCAGGCTGTG
GCTGCTGGGCGTAGCGGTGCGAATATCGCGTTTATTGCCTGTACGGGTG
ATGACAGCATTGGTGAGAGCGTTCGCCAGCAGCTCGCCACTGATAACAT
TGATATTACTCCGGTCAGCGTGATCAAAGGCGAATCAACAGGTGTGGCG
CTGATTTTTGTTAATGGCGAAGGTGAGAATGTCATCGGTATTCATGCCG
GCGCTAATGCTGCCCTTTCCCCGGCGCTGGTGGAAGCGCAACGTGAGCG
TATTGCCAACGCGTCAGCATTATTAATGCAGCTGGAATCACCACTCGAA
AGTGTGATGGCAGCGGCGAAAATCGCCCATCAAAATAAGACTATCGTTG
CGCTTAACCCGGCTCCGGCTCGCGAACTTCCTGACGAACTGCTGGCGCT
GGTGGACATTATTACGCCAAACGAAACGGAAGCAGAAAAGCTCACCGGT
ATTCGTGTTGAAAATGATGAAGATGCAGCGAAGGCGGCGCAGGTACTGC
ATGAAAAAGGTATCCGTACTGTACTGATTACTTTAGGAAGTCGTGGTGT
ATGGGCTAGCGTGAATGGTGAAGGTCAGCGCGTTCCTGGATTCCGGGTG
CAGGCTGTCGATACCATTGCTGCCGGAGATACCTTTAACGGTGCGTTAA
TCACGGCATTGCTGGAAGAAAAACCATTGCCAGAGGCGATTCGTTTTGC
CCATGCTGCCGCTGCGATTGCCGTAACACGTAAAGGCGCACAACCTTCC
GTACCGTGGCGTGAAGAGATCGACGCATTTTTAGACAGGCAGAGGTAA

In some occurrences, the at least one ribokinase enzyme is a ribokinase enzyme based on the amino acid sequences of SEQ ID NO: 32 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 32. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one ribokinase enzyme is a ribokinase enzyme encoded by the DNA sequences of SEQ ID NO: 33 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 33. These differences can be amino acid insertions, deletions, substitutions, or any combinations of such changes. In some occurrences, the sequence differences can comprise non-conservative, conservative, as well as a combination of non-conservative and conservative amino acid substitutions.

In a seventh instance of the second aspect, the at least one phosphopentomutase enzyme is one or more phosphopentomutase enzymes selected from the group consisting of wild-type phosphopentomutase enzymes and phosphopentomutase enzymes that are produced from the directed evolution from a commercially available, wild-type phosphopentomutase enzyme. In specific occurrences, the at least one phosphopentomutase enzyme is a wild-type phosphopentomutase enzyme that comprises the amino acid sequence as set forth below in SEQ ID NO: 34.

(SEQ ID NO: 34)
MKRAFIMVLDSFGIGATEDAERFGDVGADTLGHIAEACAKGEADNGRKG
PLNLPNLTRLGLAKAHEGSTGFIPAGMDGNAEVIGAYAWAHEMSSGKDT
PSGHWEIAGVPVLFEWGYFSDHENSFPQELLDKLVERANLPGYLGNCHS
SGTVILDQLGEEHMKTGKPIFYTSADSVFQIACHEETFGLDKLYELCEI
AREELTNGGYNIGRVIARPFIGDKAGNFQRTGNRHDLAVEPPAPTVLQK
LVDEKHGQVVSVGKIADIYANCGITKKVKATGLDALFDATIKEMKEAGD
NTIVFTNFVDFDSSWGHRRDVAGYAAGLELFDRRLPELMSLLRDDDILI
LTADHGCDPTWTGTDHTREHIPVLVYGPKVKPGSLGHRETFADIGQTLA
KYFGTSDMEYGKAMF

In specific occurrences, the wild-type phosphopentomutase enzyme comprising the amino acid sequence as set forth above in SEQ ID NO: 34 may be encoded by the DNA sequence as set forth below in SEQ ID NO: 35.

(SEQ ID NO: 35)
ATGAAACGTGCATTTATTATGGTGCTGGACTCATTCGGCATCGGCGCTA
CAGAAGATGCAGAACGCTTTGGTGACGTCGGGGCTGACACCCTGGGTCA
TATCGCAGAAGCTTGTGCCAAAGGCGAAGCTGATAACGGTCGTAAAGGC
CCGCTCAATCTGCCAAATCTGACCCGTCTGGGGCTGGCGAAAGCACACG
AAGGTTCTACCGGTTTCATTCCGGCGGGAATGGACGGCAACGCTGAAGT
TATCGGCGCGTACGCATGGGCGCACGAAATGTCATCCGGTAAAGATACC
CCGTCTGGTCACTGGGAAATTGCCGGTGTCCCGGTTCTGTTTGAGTGGG
GATATTTCTCCGATCACGAAAACAGCTTCCCGCAAGAGCTGCTGGATAA
ACTGGTCGAACGCGCTAATCTGCCGGGTTACCTCGGTAACTGCCACTCT
TCCGGTACGGTCATTCTGGATCAACTGGGCGAAGAGCACATGAAAACCG
GCAAGCCGATTTTCTATACCTCCGCTGACTCCGTGTTCCAGATTGCCTG
CCATGAAGAAACTTTCGGTCTGGATAAACTCTACGAACTGTGCGAAATC
GCCCGTGAAGAGCTGACCAACGGCGGCTACAATATCGGTCGTGTTATCG
CTCGTCCGTTTATCGGCGACAAAGCCGGTAACTTCCAGCGTACCGGTAA
CCGTCACGACCTGGCTGTTGAGCCGCCAGCACCGACCGTGCTGCAGAAA
CTGGTTGATGAAAAACACGGCCAGGTGGTTTCTGTCGGTAAAATTGCGG
ACATCTACGCCAACTGCGGTATCACCAAAAAAGTGAAAGCGACTGGCCT
GGACGCGCTGTTTGACGCCACCATCAAAGAGATGAAAGAAGCGGGTGAT
AACACCATCGTCTTCACCAACTTCGTTGACTTCGACTCTTCCTGGGGCC
ACCGTCGCGACGTCGCCGGTTATGCCGCGGGTCTGGAACTGTTCGACCG
CCGTCTGCCGGAGCTGATGTCTCTGCTGCGCGATGACGACATCCTGATC
CTCACCGCTGACCACGGTTGCGATCCGACCTGGACCGGTACTGACCACA
CGCGTGAACACATTCCGGTACTGGTATATGGCCCGAAAGTAAAACCGGG
CTCACTGGGTCATCGTGAAACCTTCGCGGATATCGGCCAGACTCTGGCA
AAATATTTTGGTACTTCTGATATGGAATATGGCAAAGCCATGTTCTAA

In specific occurrences of this seventh instance, the at least one phosphopentomutase enzyme is a phosphopentomutase enzyme comprising the amino acid sequence as set forth below in SEQ ID NO: 36.

(SEQ ID NO: 36)
MKRAFIMVLDSFGIGATEDAERFGDVGADTLGHIAEACAKGEADNGRKG
PLNLPNLTRLGLAKAHEGSTGFIPAGMDGNAEVIGAYAWAHEMSSGKDT
PSGHWEIAGVPVLFEWGYFSDHENSFPQELLDKLVERANLPGYLGNCHS
SGTVILDQLGEEHMKTGKPIFYTSADSVFQIACHEETFGLDKLYELCEI
AREELTNGGYNIGRVIARPFIGDKAGNFQRTGNRRDLAVEPPAPTVLQK
LVDEKHGQVVSVGKIADIYANCGITKKVKATGLDALFDATIKEMKEAGD
NTIVFTNFVDFDSSWGHRRDVAGYAAGLELFDRRLPELMSLLRDDDILI
LTADHGCDPTWTGTDHTREHIPVLVYGPKVKPGSLGHRETFADIGQTLA
KYFGTSDMEYGKAMFHHHHHH

In specific examples of such occurrences, the phosphopentomutase enzyme comprising the amino acid sequence as set forth above in SEQ ID NO: 36 may be encoded by the DNA sequence as set forth below in SEQ ID NO: 37.

(SEQ ID NO: 37)
ATGAAACGTGCATTTATTATGGTGCTGGACTCATTCGGCATCGGCGCTA
CAGAAGATGCAGAACGCTTTGGTGACGTCGGGGCTGACACCCTGGGTCA
TATCGCAGAAGCTTGTGCCAAAGGCGAAGCTGATAACGGTCGTAAAGGC
CCGCTCAATCTGCCAAATCTGACCCGTCTGGGGCTGGCGAAAGCACACG
AAGGTTCTACCGGTTTCATTCCGGCGGGAATGGACGGCAACGCTGAAGT
TATCGGCGCGTACGCATGGGCGCACGAAATGTCATCCGGTAAAGATACC
CCGTCTGGTCACTGGGAAATTGCCGGTGTCCCGGTTCTGTTTGAGTGGG
GATATTTCTCCGATCACGAAAACAGCTTCCCGCAAGAGCTGCTGGATAA
ACTGGTCGAACGCGCTAATCTGCCGGGTTACCTCGGTAACTGCCACTCT
TCCGGTACGGTCATTCTGGATCAACTGGGCGAAGAGCACATGAAAACCG
GCAAGCCGATTTTCTATACCTCCGCTGACTCCGTGTTCCAGATTGCCTG
CCATGAAGAAACTTTCGGTCTGGATAAACTCTACGAACTGTGCGAAATC
GCCCGTGAAGAGCTGACCAACGGCGGCTACAATATCGGTCGTGTTATCG
CTCGTCCGTTTATCGGCGACAAAGCCGGTAACTTCCAGCGTACCGGTAA
CCGTCGTGACCTGGCTGTTGAGCCGCCAGCACCGACCGTGCTGCAGAAA
CTGGTTGATGAAAAACACGGCCAGGTGGTTTCTGTCGGTAAAATTGCGG
ACATCTACGCCAACTGCGGTATCACCAAAAAAGTGAAAGCGACTGGCCT
GGACGCGCTGTTTGACGCCACCATCAAAGAGATGAAAGAAGCGGGTGAT
AACACCATCGTCTTCACCAACTTCGTTGACTTCGACTCTTCCTGGGGCC
ACCGTCGCGACGTCGCCGGTTATGCCGCGGGTCTGGAACTGTTCGACCG
CCGTCTGCCGGAGCTGATGTCTCTGCTGCGCGATGACGACATCCTGATC
CTCACCGCTGACCACGGTTGCGATCCGACCTGGACCGGTACTGACCACA
CGCGTGAACACATTCCGGTACTGGTATATGGCCCGAAAGTAAAACCGGG
CTCACTGGGTCATCGTGAAACCTTCGCGGATATCGGCCAGACTCTGGCA
AAATATTTTGGTACTTCTGATATGGAATATGGCAAAGCCATGTTCCATC
ATCATCACCATCATTAA

In specific occurrences of this seventh instance, the at least one phosphopentomutase enzyme is a phosphopentomutase enzyme comprising the amino acid sequence as set forth below in SEQ ID NO: 38.

(SEQ ID NO: 38)
MKRAFIMVLDSFGIGATEDAEKFGDVGADTLGHIAEACAKGEADNGRKG
PLNVPNLTRLGLAKAHEGSTGFIPAGMDGNAEVIGAYAWAHEMSSGKDT
PSGHWEIAGVPVLFEWGYFSDLENSFPQELLDKLVERANLPGYLGNCHS
SGTVILDQLGEEHMKTGKPIFYTSADSVFQIACHEETFGLDKLYELCEI
AREELTNGGYNIGRVIARPFIGDKAGNFQRTGNRRDLAVEPPAPTVLQK
LVDEKHGQVVGVGKIADIYANCGITKKVKATGLDALFDTTIKEMKEAGD
NTIVFTNFVDFDSSWGHRRDVAGYAAGLELFDRRLPELMSLLRDDDILI
LTADHGCDPTWTGTDHTREHIPVLVYGPKVKPGSLGHRETFADIGQTLA
KYFGTSDMEYGKAMFHHHHHH

In specific examples of such occurrences, the phosphopentomutase enzyme comprising the amino acid sequence as set forth above in SEQ ID NO: 38 may be encoded by the DNA sequence as set forth below in SEQ ID NO: 39.

(SEQ ID NO: 39)
ATGAAACGTGCATTTATTATGGTGCTGGACTCATTCGGCATCGGCGCTA
CAGAAGATGCAGAAAAGTTTGGTGACGTCGGGGCTGACACCCTGGGTCA
TATCGCAGAAGCTTGTGCCAAAGGCGAAGCTGATAACGGTCGTAAAGGC
CCGCTCAATGTGCCAAATCTGACCCGTCTGGGGCTGGCGAAAGCGCACG
AAGGCTCTACCGGTTTCATTCCGGCGGGAATGGACGGCAACGCTGAAGT
TATCGGCGCGTACGCATGGGCGCACGAAATGTCATCCGGTAAAGATACC
CCGTCTGGTCACTGGGAAATTGCCGGTGTCCCGGTTCTGTTCGAGTGGG
GATATTTCTCCGATCTGGAAAACAGCTTCCCGCAAGAGCTGCTGGATAA
ACTGGTCGAACGCGCTAATCTGCCGGGTTACCTCGGTAACTGCCACTCT
TCCGGTACGGTCATTCTGGATCAACTGGGCGAAGAGCACATGAAAACCG
GCAAGCCGATTTTCTATACCTCCGCTGACTCCGTGTTCCAGATTGCCTG
CCATGAAGAAACTTTCGGTCTGGATAAACTCTACGAACTGTGCGAAATC
GCCCGTGAAGAGCTGACCAACGGCGGCTACAATATCGGTCGTGTTATCG
CTCGTCCGTTTATCGGCGACAAAGCCGGTAACTTCCAGCGTACCGGTAA
CCGTCGTGACCTGGCTGTTGAGCCGCCAGCACCGACCGTGCTGCAGAAA
CTGGTTGATGAAAAACACGGCCAGGTGGTAGGCGTCGGTAAAATTGCGG
ACATCTACGCCAACTGCGGTATCACCAAAAAAGTGAAAGCGACTGGCCT
GGACGCGCTGTTTGACACTACCATCAAAGAGATGAAAGAAGCGGGTGAT
AACACCATCGTCTTCACCAACTTCGTTGACTTCGACTCTTCCTGGGGCC
ACCGTCGCGACGTCGCCGGTTATGCCGCGGGTCTGGAACTGTTCGACCG
CCGTCTGCCGGAGCTGATGTCTCTGCTGCGCGATGACGACATCCTGATC
CTCACCGCTGACCACGGTTGCGATCCGACCTGGACCGGTACTGACCACA
CGCGTGAACACATTCCGGTACTGGTATATGGCCCGAAAGTAAAACCGGG
CTCACTGGGTCATCGTGAAACCTTCGCGGATATCGGCCAGACTCTGGCA
AAATATTTTGGTACTTCTGATATGGAATATGGCAAAGCCATGTTCCATC
ATCATCACCATCATTAA

In some occurrences, the at least one phosphopentomutase enzyme is a phosphopentomutase enzyme based on the amino acid sequences of SEQ ID NO: 34, 36, or 38 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 34, 36, or 38. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one phosphopentomutase enzyme is a phosphopentomutase enzyme encoded by the DNA sequences of SEQ ID NO: 35, 37, or 39 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 35, 37, or 39. These differences can be amino acid insertions, deletions, substitutions, or any combinations of such changes. In some occurrences, the sequence differences can comprise non-conservative, conservative, as well as a combination of non-conservative and conservative amino acid substitutions.

In an eighth instance of the second aspect, the at least one sucrose phosphorylase enzyme is one or more sucrose phosphorylase enzymes selected from the group consisting of wild-type sucrose phosphorylase enzymes and sucrose phosphorylase enzymes that are produced from the directed evolution from a commercially available, wild-type sucrose phosphorylase enzyme. In specific occurrences, the at least one sucrose phosphorylase enzyme is a wild-type sucrose phosphorylase enzyme that comprises the amino acid sequence as set forth below in SEQ ID NO: 40.

(SEQ ID NO: 40)
MKNKVQLITYADRLGDGTLKSMTETLRKHFEGVYEGVHILPFFTPFDGA
DAGFDPVDHTKVDPRLGSWDDVAELSTTHDIMVDTIVNHMSWESEQFQD
VMAKGEDSEYYPMFLTMSSIFPDGVTEEDLTAIYRPRPGLPFTHYNWGG
KTRLVWTTFTPQQVDIDTDSEMGWNYLLSILDQLSQSHVSQIRLDAVGY
GAKEKNSSCFMTPKTFKLIERIKAEGEKRGLETLIEVHSYYKKQVEIAS
KVDRVYDFAIPGLLLHALEFGKTDALAQWIDVRPNNAVNVLDTHDGIGV
IDIGSDQMDRSLAGLVPDEEVDALVESIHRNSKGESQEATGAAASNLDL
YQVNCTYYAALGSDDQKYIAARAVQFFMPGVPQVYYVGALAGSNDMDLL
KRTNVGRDINRHYYSAAEVASEVERPVVQALNALGRFRNTLSAFDGEFS
YSNADGVLTMTWADDATRATLTFAPKANSNGASVARLEWTDAAGEHATD
DLIANPPVVA

In specific occurrences of this eighth instance, the at least one sucrose phosphorylase enzyme is a sucrose phosphorylase enzyme comprising the amino acid sequence as set forth below in SEQ ID NO: 41.

(SEQ ID NO: 41)
MKNKVQLITYADRLGDGTLKSMTETLRKHFEGVYEGVHILPFFTPFDGA
DAGFDPVDHTKVDPRLGSWDDVAELSTTHDIMVDTIVNHMSWESEQFQD
VMAKGEDSEYYPMFLTMSSIFPDGVTEEDLTAIYRPRPGLPFTHYNWGG
KTRLVWTTFTRQQVDIDTDSEMGWNYLLSILDQLSQSHVSQIRLDAVGY
GAKEKNSSCFLTPKTFKLVERIKAEGEKRGLETLIEVHSYYKKQVEIAS
KVDRVYDFAIPGLLLHALEFGKTDALAQWIDVRPNNAVNVLDTHDGIGV
IDIGSDQMDRSLAGLVPDEEVDALVESIHRNSKGESQEATGAAASNLDL
YQVNCTYYAALGSDDQKYIAARAVQFFMPGVPQVYYVGALAGSNDMDLL
KRTNSGRGINRHYYSAAEVASEVERPVVQALNALGRFRNTLSAFDGEFS
YSNADGVLTMTWADDATRATLTFAPKANSNGASVARLEWTDAAGEHATD
DLIANPPVVA

In specific occurrences, the sucrose phosphorylase enzyme comprising the amino acid sequence as set forth above in SEQ ID NO: 41 may be encoded by the DNA sequence as set forth below in SEQ ID NO: 42.

(SEQ ID NO: 42)
ATGAAGAACAAAGTTCAACTGATTACCTATGCGGATCGTCTGGGTGACG
GCACCCTGAAGAGCATGACCGAGACCCTGCGTAAACACTTCGAGGGTGT
TTATGAAGGCGTGCACATCCTGCCGTTCTTTACCCCGTTCGATGGTGCG
GACGCGGGCTTTGATCCGGTTGACCACACCAAAGTGGACCCGCGTCTGG
GTAGCTGGGACGATGTGGCGGAACTGAGCACCACCCACGATATCATGGT
TGACACCATTGTGAACCACATGAGCTGGGAGAGCGAACAGTTCCAAGAT
GTTATGGCGAAGGGCGAGGACAGCGAATACTATCCGATGTTCCTGACCA
TGAGCAGCATTTTTCCGGATGGTGTGACCGAGGAAGACCTGACCGCGAT
CTATCGTCCGCGTCCGGGTCTGCCGTTCACCCACTATAACTGGGGTGGC
AAAACCCGTCTGGTTTGGACCACCTTTACCCGTCAGCAAGTGGACATTG
ATACCGACAGCGAGATGGGTTGGAACTACCTGCTGAGCATCCTGGATCA
GCTGAGCCAAAGCCACGTTAGCCAAATTCGTCTGGACGCGGTGGGTTAT
GGCGCGAAGGAGAAAAACAGCAGCTGCTTCCTGACCCCGAAGACCTTTA
AACTGGTCGAACGTATTAAGGCGGAGGGTGAAAAACGTGGCCTGGAGAC
CCTGATCGAAGTTCACAGCTACTATAAGAAACAGGTGGAGATTGCGAGC
AAGGTGGATCGTGTTTACGACTTTGCGATCCCGGGTCTGCTGCTGCATG
CGCTGGAATTTGGCAAAACCGATGCGCTGGCGCAATGGATTGACGTTCG
TCCGAACAACGCGGTGAACGTTCTGGATACCCACGACGGTATCGGCGTT
ATCGATATTGGTAGCGATCAGATGGACCGTAGCCTGGCGGGTCTGGTGC
CGGATGAGGAAGTTGACGCGCTGGTTGAGAGCATCCACCGTAACAGCAA
GGGTGAAAGCCAGGAAGCGACCGGCGCGGCGGCGAGCAACCTGGACCTG
TACCAAGTTAACTGCACCTACTATGCGGCGCTGGGTAGCGACGATCAGA
AATATATTGCGGCGCGTGCGGTGCAGTTCTTTATGCCGGGCGTGCCGCA
AGTTTACTATGTGGGTGCGCTGGCGGGCAGCAACGATATGGACCTGCTG
AAGCGTACCAACAGTGGTCGTGGCATCAACCGTCACTACTATAGCGCGG
CGGAAGTGGCGAGCGAGGTGGAACGTCCGGTGGTTCAGGCGCTGAACGC
GCTGGGCCGTTTCCGTAACACCCTGAGCGCGTTCGATGGTGAATTTAGC
TACAGCAACGCGGACGGCGTTCTGACCATGACCTGGGCGGATGATGCGA
CCCGTGCGACCCTGACCTTTGCGCCGAAGGCGAACAGCAACGGTGCGAG
CGTGGCGCGTCTGGAGTGGACCGATGCGGCGGGTGAACATGCGACCGAC
GATCTGATCGCGAACCCGCCGGTGGTTGCGTAA

In specific occurrences of this eighth instance, the at least one sucrose phosphorylase enzyme is a sucrose phosphorylase enzyme comprising the amino acid sequence as set forth below in SEQ ID NO: 43.

(SEQ ID NO: 43)
MKNKVQLITYADRLGDGTLKSMTETLRKHFEGVYEGVHILPFFTPFDGA
DAGFDPVDHTKVDPRLGSWDDVAELSTTHDIMVDTIVNHMSWESEQFQD
VMAKGEDSEYYPMFLTMSSIFPDGVTEEDLTAIYRPRPGLPFTHYNWGG
KTRLVWTTFTRQQVDIDTDSEMGWNYLLSILDQLSQSHVSQIRLDAVGY
GAKEKNSSCFLTPKTFKLVERIKAEGEKRGLETLIEVHSYYKKQVEIAS
KVDRVYDFAIPGLLLHALEFGKTDALAQWIDVRPNNAVNVLDTHDGIGV
IDIGSDQMDRSLAGLVPDEEVDALVESIHRNSKGESQEATGAAASNLDL
YQVNCTYYAALGSDDQKYIAARAVQFFMPGVPQVYYVGALAGSNDMDLL
KRTNSGRGINRHYYSAAEVASEVERPVVQALNALGRFRNTLSAFDGEFS
YSNADGVLTMTWADDATRATLTFAPKANSNGASVARLEWTDAAGEHATD
DLIANPPVVAGQTGHHHHHH

In specific examples of such occurrences, the sucrose phosphorylase enzyme comprising the amino acid sequence as set forth above in SEQ ID NO: 43 may be encoded by the DNA sequence as set forth below in SEQ ID NO: 44.

(SEQ ID NO: 44)
ATGAAGAACAAAGTTCAACTGATTACCTATGCGGATCGTCTGGGTGACG
GCACCCTGAAGAGCATGACCGAGACCCTGCGTAAACACTTCGAGGGTGT
TTATGAAGGCGTGCACATCCTGCCGTTCTTTACCCCGTTCGATGGTGCG
GACGCGGGCTTTGATCCGGTTGACCACACCAAAGTGGACCCGCGTCTGG
GTAGCTGGGACGATGTGGCGGAACTGAGCACCACCCACGATATCATGGT
TGACACCATTGTGAACCACATGAGCTGGGAGAGCGAACAGTTCCAAGAT
GTTATGGCGAAGGGCGAGGACAGCGAATACTATCCGATGTTCCTGACCA
TGAGCAGCATTTTTCCGGATGGTGTGACCGAGGAAGACCTGACCGCGAT
CTATCGTCCGCGTCCGGGTCTGCCGTTCACCCACTATAACTGGGGTGGC
AAAACCCGTCTGGTTTGGACCACCTTTACCCGTCAGCAAGTGGACATTG
ATACCGACAGCGAGATGGGTTGGAACTACCTGCTGAGCATCCTGGATCA
GCTGAGCCAAAGCCACGTTAGCCAAATTCGTCTGGACGCGGTGGGTTAT
GGCGCGAAGGAGAAAAACAGCAGCTGCTTCCTGACCCCGAAGACCTTTA
AACTGGTCGAACGTATTAAGGCGGAGGGTGAAAAACGTGGCCTGGAGAC
CCTGATCGAAGTTCACAGCTACTATAAGAAACAGGTGGAGATTGCGAGC
AAGGTGGATCGTGTTTACGACTTTGCGATCCCGGGTCTGCTGCTGCATG
CGCTGGAATTTGGCAAAACCGATGCGCTGGCGCAATGGATTGACGTTCG
TCCGAACAACGCGGTGAACGTTCTGGATACCCACGACGGTATCGGCGTT
ATCGATATTGGTAGCGATCAGATGGACCGTAGCCTGGCGGGTCTGGTGC
CGGATGAGGAAGTTGACGCGCTGGTTGAGAGCATCCACCGTAACAGCAA
GGGTGAAAGCCAGGAAGCGACCGGCGCGGCGGCGAGCAACCTGGACCTG
TACCAAGTTAACTGCACCTACTATGCGGCGCTGGGTAGCGACGATCAGA
AATATATTGCGGCGCGTGCGGTGCAGTTCTTTATGCCGGGCGTGCCGCA
AGTTTACTATGTGGGTGCGCTGGCGGGCAGCAACGATATGGACCTGCTG
AAGCGTACCAACAGTGGTCGTGGCATCAACCGTCACTACTATAGCGCGG
CGGAAGTGGCGAGCGAGGTGGAACGTCCGGTGGTTCAGGCGCTGAACGC
GCTGGGCCGTTTCCGTAACACCCTGAGCGCGTTCGATGGTGAATTTAGC
TACAGCAACGCGGACGGCGTTCTGACCATGACCTGGGCGGATGATGCGA
CCCGTGCGACCCTGACCTTTGCGCCGAAGGCGAACAGCAACGGTGCGAG
CGTGGCGCGTCTGGAGTGGACCGATGCGGCGGGTGAACATGCGACCGAC
GATCTGATCGCGAACCCGCCGGTGGTTGCGGGCCAAACTGGCCACCATC
ACCATCACCATTAGTAA

In some occurrences, the at least one sucrose phosphorylase enzyme is a sucrose phosphorylase enzyme based on the amino acid sequences of SEQ ID NO: 40, 41, or 43 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 40, 41, or 43. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one sucrose phosphorylase enzyme is a sucrose phosphorylase enzyme encoded by the DNA sequences of SEQ ID NO: 42 or 44 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 42 or 44. These differences can be amino acid insertions, deletions, substitutions, or any combinations of such changes. In some occurrences, the sequence differences can comprise non-conservative, conservative, as well as a combination of non-conservative and conservative amino acid substitutions.

In a third aspect of this sixth embodiment, the reacting is conducted in the presence of at least one solvent. In instances of this aspect, the at least one solvent is selected from aqueous solvents, organic solvents and mixtures thereof. In particular instances of this aspect, the at least one solvent is selected from the group consisting of water. In other particular instances of this aspect, the at least one solvent is selected from the group consisting of DME, anisole, tert-butanol, tert-amyl alcohol, acetone, 1,2-propylene carbonate, 1,3-dioxolane, acetonitrile, ethyl acetate, methyl tert-butyl ether, cyclopentyl methyl ether, chlorobenzene, isopropanol, isopropyl acetate, 2-butanone, and mixtures thereof. In still further instances of this aspect, the at least one solvent is water and at least one organic solvent selected from the group consisting of DME, anisole, tert-butanol, tert-amyl alcohol, acetone, 1,2-propylene carbonate, 1,3-dioxolane, acetonitrile, ethyl acetate, methyl tert-butyl ether, cyclopentyl methyl ether, chlorobenzene, isopropanol, isopropyl acetate, 2-butanone, and mixtures thereof.

A seventh embodiment of the processes of the disclosure comprises (a) reacting ribose with uracil in the presence of at least one enzyme to form uridine; (b) reacting uridine with at least one isobutyryl donor in the presence of at least one lipase enzyme to form 5′-isobutyryluridine; and (c) reacting 5′-isobutyryluridine with at least one hydroxylamine source in the presence of at least one activating agent to produce Compound B:

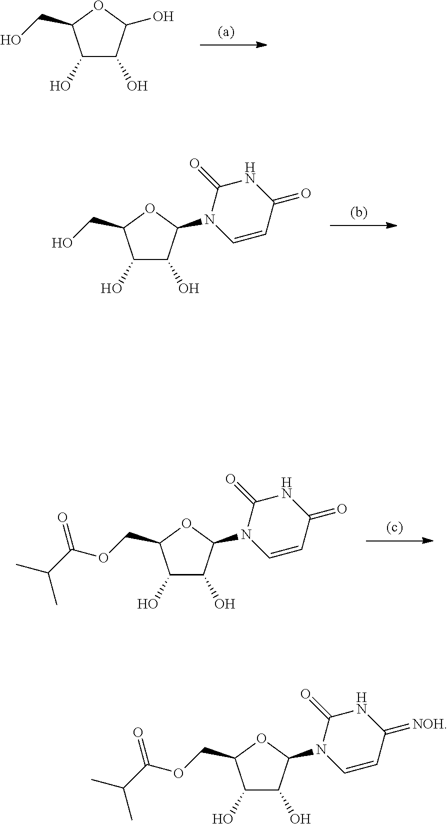

In a first aspect of this seventh embodiment, uracil is provided in an amount in a range of from about 0.5 to about 1.2 equivalents with respect to the amount of ribose, such as an amount of about 0.8 equivalents.

In a second aspect of this seventh embodiment, the at least one enzyme is selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme, at least one phosphopentomutase enzyme, and at least one sucrose phosphorylase enzyme, and mixtures thereof.

In instances of this second aspect, the at least one enzyme is a mixture of at least two enzymes selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme, at least one phosphopentomutase enzyme, and at least one sucrose phosphorylase enzyme. In some instances, the at least one enzyme is a mixture of at least three enzymes selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme, at least one phosphopentomutase enzyme, and at least one sucrose phosphorylase enzyme. In further instances, the at least one enzyme is a mixture of at least four enzymes selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme, at least one phosphopentomutase enzyme, and at least one sucrose phosphorylase enzyme. In still further instances, the at least one enzyme is a mixture of at least five enzymes selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme, at least one phosphopentomutase enzyme, and at least one sucrose phosphorylase enzyme. In particular instances, the at least one enzyme is a mixture of at least one S-methyl-5-thioribose kinase enzyme, at least one acetate kinase enzyme, at least one pyruvate oxidase enzyme, at least one catalase enzyme, and at least one uridine phosphorylase enzyme. In other particular instances, the at least one enzyme is a mixture of at least one S-methyl-5-thioribose kinase enzyme, at least one acetate kinase enzyme, at least one uridine phosphorylase enzyme, and at least one sucrose phosphorylase enzyme. In additional particular instances, the at least one enzyme is a mixture of at least one acetate kinase enzyme, at least one pyruvate oxidase enzyme, at least one catalase enzyme, at least one uridine phosphorylase enzyme, at least one ribokinase enzyme, and at least one phosphopentomutase enzyme. In further particular instances, the at least one enzyme is a mixture of at least one S-methyl-5-thioribose kinase enzyme, at least one acetate kinase enzyme, at least one uridine phosphorylase enzyme, at least one ribokinase enzyme, at least one phosphopentomutase enzyme, and at least one sucrose phosphorylase enzyme. In still further particular instances, the at least one enzyme is a mixture of at least one acetate kinase enzyme, at least one uridine phosphorylase enzyme, and at least two enzymes selected from the group consisting of at least one S-methyl-5-thioribose kinase enzyme, at least one pyruvate oxidase enzyme, at least one catalase enzyme, at least one ribokinase enzyme, at least one phosphopentomutase enzyme, and at least one sucrose phosphorylase enzyme.

In a first instance of the second aspect of this seventh embodiment, the at least one S-methyl-5-thioribose kinase enzyme is one or more S-methyl-5-thioribose kinase enzymes selected from the group consisting of wild-type S-methyl-5-thioribose kinase enzymes and S-methyl-5-thioribose kinase enzymes that are produced from the directed evolution from a commercially available, wild-type S-methyl-5-thioribose kinase enzyme. In specific instances, the at least one S-methyl-5-thioribose kinase enzyme is selected from the group consisting of S-methyl-5-thioribose kinase enzymes that are produced from the directed evolution from a commercially available, wild-type S-methyl-5-thioribose kinase enzyme, which has the amino acid sequence as set forth above in SEQ ID NO: 1; in specific occurrences, the wild-type S-methyl-5-thioribose kinase enzyme comprising the amino acid sequence set forth above in SEQ ID NO: 1 may be encoded by the DNA sequence as set forth above in SEQ ID NO: 2. In specific occurrences of this first instance, the at least one S-methyl-5-thioribose kinase enzyme is a S-methyl-5-thioribose kinase enzyme comprising the amino acid sequence as set forth above in SEQ ID NO: 3. In specific examples of such occurrences, the S-methyl-5-thioribose kinase enzyme comprising the amino acid sequence as set forth above in SEQ ID NO: 3 may be encoded by the DNA sequence as set forth above in SEQ ID NO: 4. In specific occurrences of this first instance, the at least one S-methyl-5-thioribose kinase enzyme is a S-methyl-5-thioribose kinase enzyme comprising the amino acid sequence as set forth above in SEQ ID NO: 5. In specific examples of such occurrences, the S-methyl-5-thioribose kinase enzyme comprising the amino acid sequence as set forth above in SEQ ID NO: 5 may be encoded by the DNA sequence as set forth above in SEQ ID NO: 6. In specific occurrences of this instance, the at least one S-methyl-5-thioribose kinase enzyme is a S-methyl-5-thioribose kinase enzyme comprising the amino acid sequence as set forth above in SEQ ID NO: 7. In specific examples of such occurrences, the S-methyl-5-thioribose kinase enzyme comprising the amino acid sequence as set forth above in SEQ ID NO: 7 may be encoded by the DNA sequence as set forth above in SEQ ID NO: 8. In specific occurrences of this instance, the at least one S-methyl-5-thioribose kinase enzyme is a S-methyl-5-thioribose kinase enzyme comprising the amino acid sequence as set forth above in SEQ ID NO: 9. In specific examples of such occurrences, the S-methyl-5-thioribose kinase enzyme comprising the amino acid sequence as set forth above in SEQ ID NO: 9 may be encoded by the DNA sequence as set forth above in SEQ ID NO: 10. In specific occurrences of this instance, the at least one S-methyl-5-thioribose kinase enzyme is a S-methyl-5-thioribose kinase enzyme comprising the amino acid sequence as set forth above in SEQ ID NO: 11. In specific examples of such occurrences, the S-methyl-5-thioribose kinase enzyme comprising the amino acid sequence as set forth above in SEQ ID NO: 11 may be encoded by the DNA sequence as set forth above in SEQ ID NO: 12. In specific occurrences of this instance, the at least one S-methyl-5-thioribose kinase enzyme is a S-methyl-5-thioribose kinase enzyme comprising the amino acid sequence as set forth above in SEQ ID NO: 13.

In some occurrences, the at least one enzyme is a S-methyl-5-thioribose kinase enzyme based on the amino acid sequences of SEQ ID NO: 1, 3, 5, 7, 9, 11, 12, or 13 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 1, 3, 5, 7, 9, 11, 12, or 13. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one enzyme is a S-methyl-5-thioribose kinase enzyme encoded by the DNA sequences of SEQ ID NO: 2, 4, 6, 8, or 10 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 2, 4, 6, 8, or 10. These differences can be amino acid insertions, deletions, substitutions, or any combinations of such changes. In some occurrences, the sequence differences can comprise non-conservative, conservative, as well as a combination of non-conservative and conservative amino acid substitutions.

In a second instance of the second aspect of this seventh embodiment, the at least one acetate kinase enzyme is one or more acetate kinase enzymes selected from the group consisting of wild-type acetate kinase enzymes and acetate kinase enzymes that are produced from the directed evolution from a commercially available, wild-type acetate kinase enzyme. In specific instances, the at least one acetate kinase enzyme is selected from the group consisting of acetate kinase enzymes that are produced from the directed evolution from a commercially available, wild-type acetate kinase enzyme, which has the amino acid sequence as set forth above in SEQ ID NO: 14. In specific occurrences, the wild-type acetate kinase enzyme comprising the amino acid sequence as set forth above in SEQ ID NO: 14 may be encoded by the DNA sequence as set forth above in SEQ ID NO: 15. In specific occurrences of this second instance, the at least one acetate kinase enzyme is an acetate kinase enzyme comprising the amino acid sequence as set forth above in SEQ ID NO: 16. In specific examples of such occurrences, the acetate kinase enzyme comprising the amino acid sequence as set forth above in SEQ ID NO: 16 may be encoded by the DNA sequence as set forth above in SEQ ID NO: 17.

In some occurrences of this second instance, the at least one acetate kinase enzyme is an acetate kinase enzyme based on the amino acid sequences of SEQ ID NO: 14 or 16 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 14 or 16. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences of this second instance, the at least one acetate kinase enzyme is an acetate kinase enzyme encoded by the DNA sequences of SEQ ID NO: 15 or 17 and can comprise an amino acid sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 15 or 17. These differences can be amino acid insertions, deletions, substitutions, or any combinations of such changes. In some occurrences, the sequence differences can comprise non-conservative, conservative, as well as a combination of non-conservative and conservative amino acid substitutions.

In a third instance of the second aspect of this seventh embodiment, the at least one pyruvate oxidase enzyme is one or more pyruvate oxidase enzymes selected from the group consisting of wild-type pyruvate oxidase enzymes and pyruvate oxidase enzymes that are produced from the directed evolution from a commercially available, wild-type pyruvate oxidase enzyme. In specific instances, the at least one pyruvate oxidase enzyme is selected from the group consisting of pyruvate oxidase enzymes that are produced from the directed evolution from a commercially available, wild-type pyruvate oxidase enzyme, which has the amino acid sequence as set forth above in SEQ ID NO: 18. In specific occurrences, the wild-type pyruvate oxidase enzyme having the amino acid sequence as set forth above in SEQ ID NO: 18 may be encoded by the DNA sequence as set forth above in SEQ ID NO: 19. In specific occurrences of this third instance, the at least one pyruvate oxidase enzyme is a pyruvate oxidase enzyme comprising the amino acid sequence as set forth above in SEQ ID NO: 20. In specific examples of such occurrences, the pyruvate oxidase enzyme comprising the amino acid sequence as set forth above in SEQ ID NO: 20 may be encoded by the DNA sequence as set forth above in SEQ ID NO: 21. In specific occurrences of this third instance, the at least one pyruvate oxidase enzyme is a pyruvate oxidase enzyme comprising the amino acid sequence as set forth above in SEQ ID NO: 22. In specific examples of such occurrences, the pyruvate oxidase enzyme comprising the amino acid sequence as set forth above in SEQ ID NO: 22 may be encoded by the DNA sequence as set forth above in SEQ ID NO: 23. In specific occurrences of this third instance, the at least one pyruvate oxidase enzyme is a pyruvate oxidase enzyme comprising the amino acid sequence as set forth above in SEQ ID NO: 24.

In some occurrences, the at least one pyruvate oxidase enzyme is a pyruvate oxidase enzyme based on the amino acid sequences of SEQ ID NO: 18, 20, 22, or 24 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 18, 20, 22, or 24. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one pyruvate oxidase enzyme is a pyruvate oxidase enzyme encoded by the DNA sequences of SEQ ID NO: 19, 21, or 23 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 19, 21, or 23. These differences can be amino acid insertions, deletions, substitutions, or any combinations of such changes. In some occurrences, the sequence differences can comprise non-conservative, conservative, as well as a combination of non-conservative and conservative amino acid substitutions.

In a fourth instance of the second aspect of this seventh embodiment, the at least one catalase enzyme is one or more catalase enzymes selected from the group consisting of wild-type catalase enzymes and catalase enzymes that are produced from the directed evolution from a commercially available, wild-type catalase enzyme. In specific instances, the at least one catalase enzyme is selected from the group consisting of catalase enzymes that are produced from the directed evolution from a commercially available, wild-type catalase enzyme, including the catalase enzyme commercially available as product number 11650645103 from Roche Diagnostics International Ltd. In specific occurrences of this fourth instance, the at least one catalase enzyme is the wild-type catalase enzyme that comprises the amino acid sequence as set forth above in SEQ ID NO: 52. In specific examples of such occurrences, the catalase enzyme comprising the amino acid sequence as set forth above in SEQ ID NO: 52 may be encoded by the DNA sequence as set forth above in SEQ ID NO: 53. In specific examples of such occurrences, the catalase enzyme comprising the amino acid sequence as set forth above in SEQ ID NO: 52 may be encoded by the DNA sequence as set forth above in SEQ ID NO: 54.

In some occurrences, the at least one catalase enzyme is at least about 85%, 86%, 87%, 88%, 89%, 90%, 91%, 92%, 93%, 94%, 95%, 96%, 97%, 98%, or 99% identical to the reference sequence of the catalase enzyme commercially available as product number 11650645103 from Roche Diagnostics International Ltd. In some occurrences, the at least one catalase enzyme is at least about 85%, 86%, 87%, 88%, 89%, 90%, 91%, 92%, 93%, 94%, 95%, 96%, 97%, 98%, or 99% identical to the reference sequence SEQ ID NO: 52. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one catalase enzyme is encoded by DNA sequences that are at least about 85%, 86%, 87%, 88%, 89%, 90%, 91%, 92%, 93%, 94%, 95%, 96%, 97%, 98%, or 99% identical to the reference sequence of the catalase enzyme commercially available as product number 11650645103 from Roche Diagnostics International Ltd. In some occurrences, the at least one catalase enzyme is encoded by DNA sequences that are at least about 85%, 86%, 87%, 88%, 89%, 90%, 91%, 92%, 93%, 94%, 95%, 96%, 97%, 98%, or 99% identical to SEQ ID NO: 53 or 54. These differences can be amino acid insertions, deletions, substitutions, or any combinations of such changes. In some occurrences, the sequence differences can comprise non-conservative, conservative, as well as a combination of non-conservative and conservative amino acid substitutions.

In a fifth instance of the second aspect of this seventh embodiment, the at least one uridine phosphorylase enzyme is one or more uridine phosphorylase enzymes selected from the group consisting of wild-type uridine phosphorylase enzymes and uridine phosphorylase enzymes that are produced from the directed evolution from a commercially available, wild-type uridine phosphorylase enzyme. In specific instances, the at least one uridine phosphorylase enzyme is selected from the group consisting of uridine phosphorylase enzymes that are produced from the directed evolution from a commercially available, wild-type uridine phosphorylase enzyme, which has the amino acid sequence as set forth above in SEQ ID NO: 25. In specific occurrences, the wild-type uridine phosphorylase enzyme comprising the amino acid sequence as set forth above in SEQ ID NO: 25 may be encoded by the DNA sequence as set forth above in SEQ ID NO: 26. In specific occurrences of this fifth instance, the at least one uridine phosphorylase enzyme is selected from a uridine phosphorylase enzyme having the amino acid sequence as set forth above in SEQ ID NO: 27. In specific occurrences of this fifth instance, the at least one uridine phosphorylase enzyme is selected from a uridine phosphorylase enzyme having the amino acid sequence as set forth above in SEQ ID NO: 28. In specific examples of such occurrences, the uridine phosphorylase enzyme comprising the amino acid sequence as set forth above in SEQ ID NO: 28 may be encoded by the DNA sequence as set forth above in SEQ ID NO: 29. In specific instances, the at least one uridine phosphorylase enzyme is selected from a uridine phosphorylase having the amino acid sequence as set forth above in SEQ ID NO: 30. In specific instances, the at least one uridine phosphorylase enzyme is selected from a uridine phosphorylase having the amino acid sequence as set forth above in SEQ ID NO: 31. In specific instances, the at least one uridine phosphorylase enzyme is a uridine phosphorylase having the amino acid sequence as set forth above in SEQ ID NO: 45. In specific instances, the at least one uridine phosphorylase enzyme is a uridine phosphorylase having the amino acid sequence as set forth above in SEQ ID NO: 46. In specific instances, the at least one uridine phosphorylase enzyme is a uridine phosphorylase having the amino acid sequence as set forth above in SEQ ID NO: 47. In specific instances, the at least one uridine phosphorylase enzyme is a uridine phosphorylase having the amino acid sequence as set forth above in SEQ ID NO: 48. In specific instances, the at least one uridine phosphorylase enzyme is a uridine phosphorylase having the amino acid sequence as set forth above in SEQ ID NO: 49. In specific instances, the at least one uridine phosphorylase enzyme is a uridine phosphorylase having the amino acid sequence as set forth above in SEQ ID NO: 50. In specific instances, the at least one uridine phosphorylase enzyme is a uridine phosphorylase having the amino acid sequence as set forth above in SEQ ID NO: 51.

In some occurrences, the at least one uridine phosphorylase enzyme is a uridine phosphorylase enzyme based on the amino acid sequences of SEQ ID NO: 25, 27, 28, 30, 31, 45, 46, 47, 48, 49, 50, or 51 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 25, 27, 28, 30, 31, 45, 46, 47, 48, 49, 50, or 51. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one uridine phosphorylase enzyme is a uridine phosphorylase enzyme encoded by the DNA sequences of SEQ ID NO: 26 or 29 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 26 or 29. These differences can be amino acid insertions, deletions, substitutions, or any combinations of such changes. In some occurrences, the sequence differences can comprise non-conservative, conservative, as well as a combination of non-conservative and conservative amino acid substitutions.

In a sixth instance of the second aspect, the at least one ribokinase enzyme is one or more ribokinase enzymes selected from the group consisting of wild-type ribokinase enzymes and ribokinase enzymes that are produced from the directed evolution from a commercially available, wild-type ribokinase enzyme. In specific occurrences, the at least one ribokinase enzyme is a wild-type ribokinase enzyme that comprises the amino acid sequence as set forth above in SEQ ID NO: 32. In specific occurrences, the wild-type ribokinase enzyme comprising the amino acid sequence set forth above as SEQ ID NO: 32 may be encoded by the DNA sequence as set forth above in SEQ ID NO: 33.

In some occurrences, the at least one ribokinase enzyme is a ribokinase enzyme based on the amino acid sequences of SEQ ID NO: 32 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 32. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one ribokinase enzyme is a ribokinase enzyme encoded by the DNA sequences of SEQ ID NO: 33 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 33. These differences can be amino acid insertions, deletions, substitutions, or any combinations of such changes. In some occurrences, the sequence differences can comprise non-conservative, conservative, as well as a combination of non-conservative and conservative amino acid substitutions.

In a seventh instance of the second aspect, the at least one phosphopentomutase enzyme is one or more phosphopentomutase enzymes selected from the group consisting of wild-type phosphopentomutase enzymes and phosphopentomutase enzymes that are produced from the directed evolution from a commercially available, wild-type phosphopentomutase enzyme. In specific occurrences, the at least one phosphopentomutase enzyme is a wild-type phosphopentomutase enzyme that comprises the amino acid sequence as set forth above in SEQ ID NO: 34. In specific occurrences, the wild-type phosphopentomutase enzyme comprising the amino acid sequence as set forth above in SEQ ID NO: 34 may be encoded by the DNA sequence as set forth above in SEQ ID NO: 35. In specific occurrences of this seventh instance, the at least one phosphopentomutase enzyme is a phosphopentomutase enzyme comprising the amino acid sequence as set forth above in SEQ ID NO: 36. In specific examples of such occurrences, the phosphopentomutase enzyme comprising the amino acid sequence as set forth above in SEQ ID NO: 36 may be encoded by the DNA sequence as set forth above in SEQ ID NO: 37. In specific occurrences of this seventh instance, the at least one phosphopentomutase enzyme is a phosphopentomutase enzyme comprising the amino acid sequence as set forth above in SEQ ID NO: 38. In specific examples of such occurrences, the phosphopentomutase enzyme comprising the amino acid sequence as set forth above in SEQ ID NO: 38 may be encoded by the DNA sequence as set forth above in SEQ ID NO: 39.

In some occurrences, the at least one phosphopentomutase enzyme is a phosphopentomutase enzyme based on the amino acid sequences of SEQ ID NO: 34, 36, or 38 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 34, 36, or 38. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one phosphopentomutase enzyme is a phosphopentomutase enzyme encoded by the DNA sequences of SEQ ID NO: 35, 37, or 39 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 35, 37, or 39. These differences can be amino acid insertions, deletions, substitutions, or any combinations of such changes. In some occurrences, the sequence differences can comprise non-conservative, conservative, as well as a combination of non-conservative and conservative amino acid substitutions.

In an eighth instance of the second aspect, the at least one sucrose phosphorylase enzyme is one or more sucrose phosphorylase enzymes selected from the group consisting of wild-type sucrose phosphorylase enzymes and sucrose phosphorylase enzymes that are produced from the directed evolution from a commercially available, wild-type sucrose phosphorylase enzyme. In specific occurrences, the at least one sucrose phosphorylase enzyme is a wild-type sucrose phosphorylase enzyme that comprises the amino acid sequence as set forth above in SEQ ID NO: 40. In specific occurrences of this eighth instance, the at least one sucrose phosphorylase enzyme is a sucrose phosphorylase enzyme comprising the amino acid sequence as set forth above in SEQ ID NO: 41. In specific occurrences, the sucrose phosphorylase enzyme comprising the amino acid sequence as set forth above in SEQ ID NO: 41 may be encoded by the DNA sequence as set forth above in SEQ ID NO: 42. In specific occurrences of this eighth instance, the at least one sucrose phosphorylase enzyme is a sucrose phosphorylase enzyme comprising the amino acid sequence as set forth above in SEQ ID NO: 43. In specific examples of such occurrences, the sucrose phosphorylase enzyme comprising the amino acid sequence as set forth above in SEQ ID NO: 43 may be encoded by the DNA sequence as set forth above in SEQ ID NO: 44.

In some occurrences, the at least one sucrose phosphorylase enzyme is a sucrose phosphorylase enzyme based on the amino acid sequences of SEQ ID NO: 40, 41, or 43 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 40, 41, or 43. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one sucrose phosphorylase enzyme is a sucrose phosphorylase enzyme encoded by the DNA sequences of SEQ ID NO: 42 or 44 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 42 or 44. These differences can be amino acid insertions, deletions, substitutions, or any combinations of such changes. In some occurrences, the sequence differences can comprise non-conservative, conservative, as well as a combination of non-conservative and conservative amino acid substitutions.

In a third aspect of this seventh embodiment, the (a) reacting is conducted in the presence of at least one solvent. In instances of this aspect, the at least one solvent is selected from aqueous solvents, organic solvents and mixtures thereof. In particular instances of this aspect, the at least one solvent is selected from the group consisting of water. In other particular instances of this aspect, the at least one solvent is selected from the group consisting of DME, anisole, tert-butanol, tert-amyl alcohol, acetone, 1,2-propylene carbonate, 1,3-dioxolane, acetonitrile, ethyl acetate, methyl tert-butyl ether, cyclopentyl methyl ether, chlorobenzene, isopropanol, isopropyl acetate, 2-butanone, and mixtures thereof. In still further instances of this aspect, the at least one solvent is water and at least one organic solvent selected from the group consisting of DME, anisole, tert-butanol, tert-amyl alcohol, acetone, 1,2-propylene carbonate, 1,3-dioxolane, acetonitrile, ethyl acetate, methyl tert-butyl ether, cyclopentyl methyl ether, chlorobenzene, isopropanol, isopropyl acetate, 2-butanone, and mixtures thereof.

In a fourth aspect of this seventh embodiment, the at least one isobutyryl donor is selected from the group consisting of propan-2-one O-isobutyryl oxime, isobutyric anhydride, and mixtures thereof. In instances of this fourth aspect, the at least one isobutyryl donor is isobutyric anhydride. In specific instances of this aspect, the at least one isobutyryl donor is provided in an amount in a range of from about 1.0 to about 3.0 equivalents with respect to the amount of ribose, such as an amount of about 1.5 equivalents.

In a fifth aspect of this seventh embodiment, the at least one lipase enzyme is selected from the group consisting of wild-type lipase enzymes and lipase enzymes that are produced from the directed evolution from a commercially available, wild-type lipase enzyme. In instance of this aspect, the at least one lipase enzyme is selected from the group consisting of IMMTLL (commercially available as IMMTLL-T2-150, from ChiralVision), IMMRES (commercially available as IMMRES-T2-150, from ChiralVision), IMMLIPX (commercially available as IMMLIPX-T2-150, from ChiralVision), IMMP6-T2-250 (commercially available as IMMP6-T2-250, from ChiralVision), Novozym® 51032 (commercially available as catalog number 06-3135, from Strem Chemicals, Inc.), and Novozym® 435 (commercially available as catalog number 3925009-810, from Novozymes, or catalog number 06-3123, from Strem Chemicals, Inc.). In specific instances, the at least one lipase enzyme is Novozym® 435.

In some occurrences, the at least one lipase enzyme is a lipase enzyme that is at least about 85%, 86%, 87%, 88%, 89%, 90%, 91%, 92%, 93%, 94%, 95%, 96%, 97%, 98%, or 99% identical to the reference sequence of the commercially available, wild-type lipase enzyme. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one lipase enzyme is a lipase enzyme encoded by DNA sequences that are at least about 85%, 86%, 87%, 88%, 89%, 90%, 91%, 92%, 93%, 94%, 95%, 96%, 97%, 98%, or 99% identical to the reference sequence of the commercially available, wild-type lipase enzyme. These differences can be amino acid insertions, deletions, substitutions, or any combinations of such changes. In some occurrences, the sequence differences can comprise non-conservative, conservative, as well as a combination of non-conservative and conservative amino acid substitutions.

In a sixth aspect of this seventh embodiment, the (b) reacting is conducted in the presence of at least one solvent. In instances of this aspect, the at least one solvent is selected from organic solvents and mixtures thereof. In particular instances of this aspect, the solvent is selected from the group consisting of DME, anisole, tert-butanol, tert-amyl alcohol, acetone, 1,2-propylene carbonate, 1,3-dioxolane, acetonitrile, ethyl acetate, methyl tert-butyl ether, cyclopentyl methyl ether, chlorobenzene, isopropanol, isopropyl acetate, 2-butanone, and mixtures thereof. In more particular instances, the at least one solvent is selected from the group consisting of tert-butanol, tert-amyl alcohol, acetone, and mixtures thereof. In still more particular instances, the at least one solvent is acetone.

In a seventh aspect of this seventh embodiment, the at least one hydroxylamine source is selected from the group consisting of hydroxylamine sulfate, hydroxylamine HCl, NH₂OH, and mixtures thereof. In instances of this seventh aspect, the at least one hydroxylamine source is hydroxylamine sulfate. In specific instances of this aspect, the at least one hydroxylamine source is provided in an amount to provide hydroxylamine in a range of from about 1.0 to about 2.5 equivalents of hydroxylamine with respect to the amount of 5′-isobutyryluridine, such as an amount in a range of from about 2.2 to about 2.5 equivalents, or an amount of about 2.5 equivalents.

In an eighth aspect of this seventh embodiment, the at least one activating agent is hexamethyldisilazane. In specific instances of this aspect, the at least one activating agent is provided in an amount in a range of from about 6.0 to about 8.0 equivalents with respect to the amount of 5′-isobutyryluridine, such as an amount of about 8 equivalents.

In a ninth aspect of this seventh embodiment, the (c) reacting is conducted in the presence of at least one acidic additive. In instances of this ninth aspect, the at least one acidic additive is selected from the group consisting of ammonium bisulfate, ammonium dihydrogen phosphate, sulfuric acid, sodium bisulfate, potassium bisulfate, imidazole bisulfate, triethylamine bisulfate, N-methylmorpholine bisulfate, N-methylimidazole bisulfate, sulfur trioxide pyridine complex, trifluoromethanesulfonic acid, methanesulfonic acid, and mixtures thereof. In specific instances, the at least one acidic additive is ammonium bisulfate. In specific instances, the at least one acidic additive is present in an amount in a range of from about 1.5 to about 3.0 equivalents with respect to the amount of 5′-isobutyryluridine.

In a tenth aspect of this seventh embodiment, the (c) reacting is optionally conducted in the presence of at least one catalyst. In certain instances of this aspect, the at least one catalyst is selected from Lewis basic catalysts. In instances of this tenth aspect, the at least one catalyst is selected from the group consisting of N-methylimidazole, N-methylmorpholine, 1,2,4-triazole, 5-(ethylthio)-1H-tetrazole, imidazole, and mixtures thereof. In specific instances, the at least one catalyst is imidazole. In specific instances, the at least one catalyst is present in an amount in a range of from about 0.2 to about 0.5 equivalents with respect to the amount of 5′-isobutyryluridine.

In an eleventh aspect of this seventh embodiment, the reacting is conducted in the presence of at least one solvent. In instances of this aspect, the at least one solvent is selected from organic solvents and mixtures thereof. In particular instances of this aspect, the at least one solvent is selected from the group consisting of heptane, toluene, 2-methyltetrahydrofuran, tetrahydrofuran, DME, sulfolane, and mixtures thereof.

In an eighth embodiment, the disclosure provides compounds selected from the group consisting of:

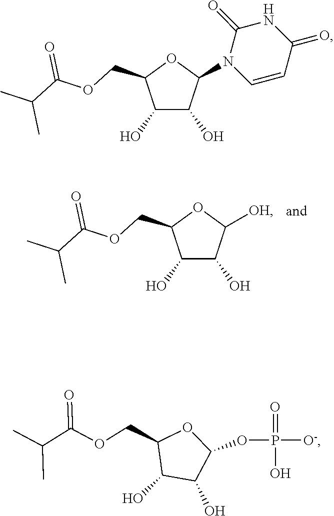

and salts thereof.

### EXAMPLES

### Example 1: Synthesis of 5-isobutyrylribose

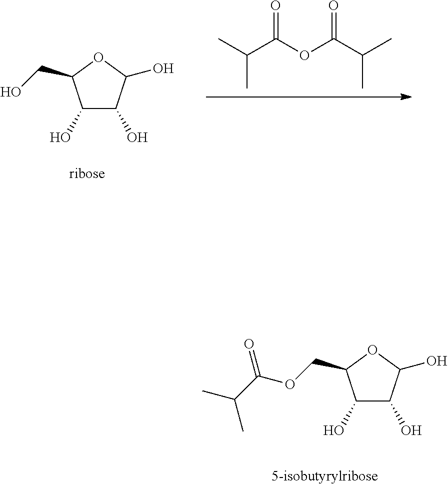

To a 2 L vessel is charged ribose (50 g), Novozym® 435 (5 g, commercially available from Novozymes (catalog #3925009-810)), acetone (1 L), and isobutyric anhydride (2.0 equiv). The contents were heated and aged at 50° C. for 12 h. The mixture was cooled, filtered, and the solid was rinsed with acetone. The filtrate was concentrated under vacuum to approximately 150 mL total volume. MTBE (200 mL) was added, and the organic solution was extracted four times each with 100 mL of water. The aqueous mixture was partially concentrated under vacuum to a final weight of 332 g.

### Example 2: Synthesis of 5′-isobutyryluridine

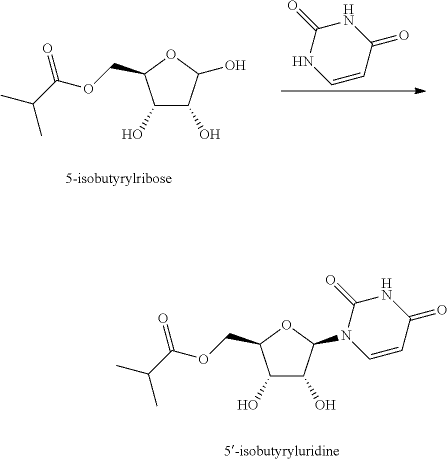 [SMILES: CC(C)C(=O)OC[C@H]1OC(O)[C@H](O)[C@@H]1O ; O=c1cc[nH]c(=O)[nH]1 ; CC(C)C(=O)OC[C@H]1O[C@@H](n2ccc(=O)[nH]c2=O)[C@H](O)[C@@H]1O]

In a reaction vessel, approximately 20 mL of water was charged, followed by addition of pyruvic acid (2.455 g), KH₂PO₄ (0.777 g), and MgCl₂-6H₂O (102 mg). pH of the resulting solution was adjusted with 50 wt % aqueous KOH (approximately 3.5 mL) to 7.1. To the mixture was charged 5′-isobutyryl ribose (4.912 g), and total volume was adjusted to approximately 45 mL. Further, thiamine pyrophosphate (51.4 mg), adenosine triphosphate disodium salt hydrate (67.5 mg), and flavin adenine dinucleotide disodium salt (9.3 mg), and antifoam (35 ul) were charged to the mixture. pH of the resulting solution was adjusted with 50 wt % aqueous KOH to 7.0. In another bottle were charged MTR-kinase of SEQ ID NO: 9 (375 mg), the acetate kinase of SEQ ID NO: 16 (12.5 mg), pyruvate oxidase of SEQ ID NO: 18 (12.5 mg), catalase (12.5 mL, commercially available as product number 11650645103 from Roche Diagnostics International Ltd.), followed by addition of 5 mL of 100 mM triethanolamine aqueous buffer with 10 mM MgCl₂ (pH 7.0) to prepare an aqueous solution of enzyme mixture. In a 100 mL reactor was added uracil (1.78 g), and uridine phosphorylase of SEQ ID NO: 28 (356 mg), followed by 45 mL of solution prepared above. The mixture was stirred by using an overhead stirrer at 25° C., followed by addition of the enzyme solution (5 mL). The mixture was agitated at 25° C. while air was sparged through a tubing to supply oxygen in the solution for 64 h. The solution was checked by HPLC to show the formation of 5′-isobutyryl uridine.

The mixture was transferred to a round bottom flask with CELITE® diatomaceous earth (3 g), MeTHF (45 mL), and ammonium sulfate (15 g), and heated at 75° C. for 30 min. The mixture was cooled to ambient temperature, filtered, and the aqueous phase was discarded. The organic phase was washed twice with water (2 mL each). The solvent was exchanged to EtOAc and heptane was added to crystallize the product, then aged at 0° to 5° C. The crystalline product was collected and washed with a 1:1.5 mixture of EtOAc:heptane to afford ((2R,3S,4R,5R)-5-(2,4-dioxo-3,4-dihydropyrimidin-1(2H)-yl)-3,4-dihydroxytetrahydrofuran-2-yl)methyl isobutyrate (4.1 g).

### Example 3: Synthesis of Uridine 4-oxime 5′-(2-methylpropanoate)

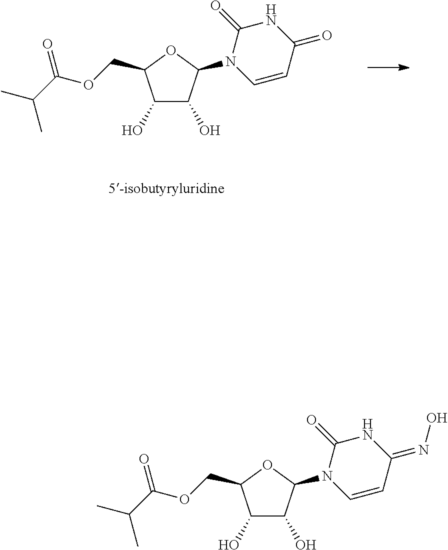 [SMILES: CC(C)C(=O)OC[C@H]1O[C@@H](n2ccc(=O)[nH]c2=O)[C@H](O)[C@@H]1O ; CC(C)C(=O)OC[C@H]1O[C@@H](n2cc/c(=N/O)[nH]c2=O)[C@H](O)[C@@H]1O]

A 100 mL vessel with overhead stirrer was set up under nitrogen. The vessel was charged with HMDS (68.0 ml, 320 mmol), and the reaction temperature was raised to 76° C. Imidazole (1.362 g, 20.00 mmol) was added, and the mixture was stirred at 78° C. for 30 min during which time all the imidazole had dissolved. Ammonium hydrogen sulfate (11.51 g, 100 mmol) was added, the temperature was adjusted to an external temperature of 75° C., and the mixture was stirred for 30 min. Hydroxylamine sulfate (8.21 g, 50.0 mmol) was added. ((2R,3S,4R,5R)-5-(2,4-dioxo-3,4-dihydropyrimidin-1(2H)-yl)-3,4-dihydroxytetrahydrofuran-2-yl)methyl isobutyrate (12.57 g, 40 mmol) was added, and the reaction mixture was aged with stirring at 75° to 85° C. for 6 h, then cooled to ambient temperature.

Water (30.00 ml) was added, and the mixture was transferred to a separatory funnel using 3 mL of water and 2 portions of heptane (5.00 ml) to complete the transfer. The aqueous phase was discarded. The organic phase was washed twice with water (10.00 ml), and the aqueous phases were discarded. Formic acid (6.00 ml, 156 mmol) was added in a single portion, and the mixture was stirred at 50° C. for 1 h. Water (25.00 ml) was added with stirring, and the mixture was transferred to a separatory funnel using water (10.00 ml) and heptane (5.00 ml). The aqueous phase was separated, and the organic phase was extracted with water (10.00 ml). The organic phase was discarded. The combined aqueous extracts were basified with ammonium hydroxide (1.01 mL, 72.0 mmol). EtOAc (50.0 ml) and ammonium sulfate (40.0 g, 302 mmol) were added, and the mixture was heated to 50° C. to give two homogeneous phases. The phases were separated at 50° C., and the aqueous phase was extracted with EtOAc (50.0 ml). The EtOAc extracts were combined and concentrated to approximately 40 mL volume. EtOAc (50.0 ml) was added, and the mixture was concentrated to approximately 40 mL volume. Again EtOAc (50.0 ml) was added, and the mixture was concentrated to approximately 40 mL volume. The resulting slurry was maintained at reflux (75° to 80° C.) for 15 min and gradually cooled to 60° C. over 30 min. MTBE (40.0 ml) was added, then the mixture was cooled to 0° C. over 2 h. The slurry was filtered, and the filter cake was washed with MTBE (40.0 ml) and dried under nitrogen stream to provide ((2R,3S,4R,5R)-3,4-dihydroxy-5-((Z)-4-(hydroxyimino)-2-oxo-3,4-dihydropyrimidin-1(2H)-yl)tetrahydrofuran-2-yl)methyl isobutyrate (11.0 g, 33.3 mmol).

### Example 4: Synthesis of 5′-isobutyryluridine from Uridine

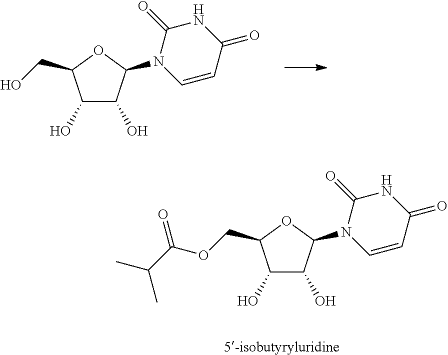 [SMILES: O=c1ccn([C@@H]2O[C@H](CO)[C@@H](O)[C@H]2O)c(=O)[nH]1 ; CC(C)C(=O)OC[C@H]1O[C@@H](n2ccc(=O)[nH]c2=O)[C@H](O)[C@@H]1O]

To a suspension of uridine (100 mg, 0.409 mmol) in DME (2000 μl) was added Novozym® 435 (5 mg, 5 wt %, commercially available from Novozymes (catalog #3925009-810)), followed by potassium phosphate monobasic (55.7 mg, 0.409 mmol). Isobutyric anhydride (64.8 mg, 0.409 mmol) was added, and the mixture was heated to 40° C. on a shaker. By 24 h, the reaction had progressed to afford 85% of 5′-isobutyryluridine.

It will be appreciated that various of the above-discussed and other features and functions, or alternatives thereof, may be desirably combined into many other different systems or applications. Also that various alternatives, modifications, variations, or improvements therein may be subsequently made by those skilled in the art that are also intended to be encompassed by the following claims.

1. A process for preparing Compound B 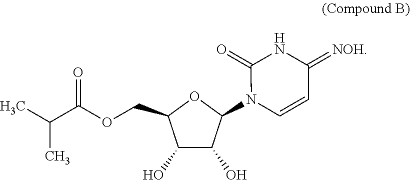
or a pharmaceutically acceptable salt, hydrate, or solvate thereof, said process comprising reacting 5′-isobutyryluridine with at least one hydroxylamine source in the presence of at least one activating agent to produce Compound B.

2. The process according to claim 1, wherein the at least one hydroxylamine source is selected from the group consisting of hydroxylamine sulfate, NH₂OH, and mixtures thereof.

3. The process according to claim 1, wherein the at least one activating agent is hexamethyldisilazane.

4. The process according to claim 1, wherein step (c) is conducted in the presence of at least one acidic additive.

5. The process according to claim 1, wherein step (c) is conducted in the presence of at least one catalyst.

6. The process according to claim 5, wherein the at least one catalyst is selected from the group consisting of N-methylimidazole, N-methylmorpholine, 1,2,4-triazole, 5-(ethylthio)-1H-tetrazole, imidazole, and mixtures thereof.

7. The process according to claim 6, wherein the at least one catalyst is imidazole.

8. The process according to claim 1, further comprising reacting 5-isobutyrylribose with uracil in the presence of at least one enzyme to form 5′-isobutyryluridine.

9. The process according to claim 8, wherein the at least one enzyme is selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, and mixtures thereof.

10. The process according to claim 8, further comprising reacting ribose with at least one isobutyryl donor in the presence of at least one lipase enzyme to form 5-isobutyrylribose.

11. The process according to claim 10, wherein the at least one isobutyryl donor is selected from the group consisting of propan-2-one O-isobutyryl oxime, isobutyric anhydride, and mixtures thereof.

12. The process according to claim 1, further comprising reacting uridine with at least one isobutyryl donor in the presence of at least one lipase enzyme to form 5′-isobutyryluridine.

13. The process according to claim 12, wherein the at least one isobutyryl donor is selected from the group consisting of propan-2-one O-isobutyryl oxime, isobutyric anhydride, and mixtures thereof.

14. The process according to claim 12, further comprises reacting ribose with uracil in the presence of at least one enzyme to form uridine.

15. The process according to claim 14, wherein the at least one enzyme is selected from the group consisting of S-methyl-5-thioribose kinase enzymes, acetate kinase enzymes, pyruvate oxidase enzymes, catalase enzymes, uridine phosphorylase enzymes, ribokinase enzymes, phosphopentomutase enzymes, and sucrose phosphorylase enzymes.

16. A process for preparing Compound B 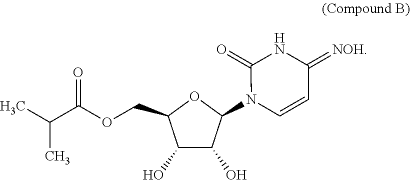
or a pharmaceutically acceptable salt, hydrate, or solvate thereof, said process comprising:
(a) reacting ribose with at least one isobutyryl donor in the presence of at least one lipase enzyme to form 5′-isobutyrylribose;
(b) reacting 5′isobutyrylribose with uracil in the presence of at least one enzyme, which is selected from the group consisting of S-methyl-5-thioribose kinase enzymes, acetate kinase enzymes, pyruvate oxidase enzymes, catalase enzymes; and uridine phosphorylase enzymes, to form 5′-isobutyryluridine; and
(c) reacting 5′-isobutyryluridine with at least one hydroxylamine source in the presence of at least one activating agent to produce Compound B;
wherein
the at least one isobutyryl donor is selected from the group consisting of propan-2-one O-isobutyryl oxime, isobutyric anhydride, and mixtures thereof,
the at least one enzyme is selected from the group consisting of S-methyl-5-thioribose kinase enzymes, acetate kinase enzymes, pyruvate oxidase enzymes, catalase enzymes; and uridine phosphorylase enzymes;
the at least one hydroxylamine source is selected from the group consisting of hydroxylamine sulfate, NH₂OH, and mixtures thereof; and
the at least one activating agent is hexamethyldisilazane.

17. The process according to claim 16, wherein step (c) is conducted in the presence of at least one acidic additive.

18. The process according to claim 16, wherein step (c) is conducted in the presence of at least one catalyst.

19. The process according to claim 18, wherein the at least one catalyst is selected from the group consisting of N-methylimidazole, N-methylmorpholine, 1,2,4-triazole, 5-(ethylthio)-1H-tetrazole, imidazole, and mixtures thereof.

20. The process according to claim 19, wherein the at least one catalyst is imidazole.

21. A process for preparing Compound B 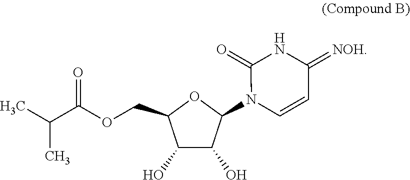
or a pharmaceutically acceptable salt, hydrate, or solvate thereof, said process comprising
(a) reacting ribose with uracil in the presence of at least one enzyme to form uridine;
(b) reacting uridine with at least one isobutyryl donor in the presence of at least one lipase enzyme to form 5′-isobutyryluridine; and
(c) reacting 5′-isobutyryluridine with at least one hydroxylamine source in the presence of at least one activating agent to produce Compound B;
wherein
the at least one enzyme is selected from the group consisting of S-methyl-5-thioribose kinase enzymes, acetate kinase enzymes, pyruvate oxidase enzymes, catalase enzymes, uridine phosphorylase enzymes, ribokinase enzymes, phosphopentomutase enzymes, and sucrose phosphorylase enzymes;
the at least one isobutyryl donor is selected from the group consisting of propan-2-one O-isobutyryl oxime, isobutyric anhydride, and mixtures thereof;
the at least one hydroxylamine source is selected from the group consisting of hydroxylamine sulfate, NH₂OH, and mixtures thereof; and
the at least one activating agent is hexamethyldisilazane.

22. The process according to claim 21, wherein step (c) is conducted in the presence of at least one acidic additive.

23. The process according to claim 21, wherein step (c) is conducted in the presence of at least one catalyst.

24. The process according to claim 21, wherein the at least one catalyst is selected from the group consisting of N-methylimidazole, N-methylmorpholine, 1,2,4-triazole, 5-(ethylthio)-1H-tetrazole, imidazole, and mixtures thereof.

25. The process according to claim 24, wherein the at least one catalyst is imidazole.

26. A compound selected from the group consisting of 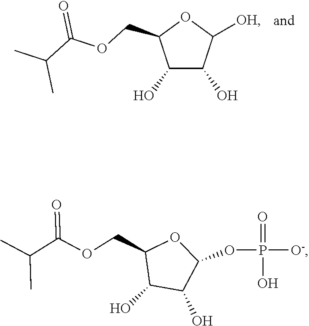
and salts thereof.


SEQUENCE LISTING (54 sequences)

SEQ ID NO: 1 - protein, 399 aa, Klebsiella sp.
MSQYHTFTAHDAVAYAQQFAGIDNPSELVSAQEVGDGNLNLVFKVFDRQGVSRAIVKQALPYVRCVGESWPLTLDRARLEAQTLVAHYQHSPQHTVKIHHFDPELAVMVMEDLSDHRIWRGELIANVYYPQAARQLGDYLAQVLFHTSDFYLHPHEKKAQVAQFINPAMCEITEDLFFNDPYQIHERNNYPAELEADVAALRDDAQLKLAVAALKHRFFAHAEALLHGDIHSGSIFVAEGSLKAIDAEFGYFGPIGFDIGTAIGNLLLNYCGLPGQLGIRDAAAAREQRLNDIHQLWTTFAERFQALAAEKTRDAALAYPGYASAFLKKVWADAVGFCGSELIRRSVGLSHVADIDTIQDDAMRHECLRHAITLGRALIVLAERIDSVDELLARVRQYS

SEQ ID NO: 2 - DNA, 1200 bp, Artificial Sequence
  Description of Artificial Sequence Synthetic polynucleotide
  Corresponding DNA seq 1
atgagccagtatcataccttcaccgcgcatgatgcggtggcgtatgcgcagcaatttgcgggcattgataacccgagcgagctggttagcgcgcaagaagttggtgacggcaacctgaacctggtgttcaaggtttttgatcgtcagggtgtgagccgtgcgatcgttaaacaagcgctgccgtacgtgcgttgcgttggtgaaagctggccgctgaccctggaccgtgcgcgtctggaagcgcagaccctggtggcgcactatcagcacagcccgcaacacaccgttaagatccaccacttcgatccggagctggcggtgatggttatggaagacctgagcgatcaccgtatttggcgtggtgagctgatcgcgaacgtgtactatccgcaggcggcgcgtcaactgggtgactacctggcgcaggttctgttccacaccagcgatttttatctgcacccgcacgagaagaaagcgcaggtggcgcaattcattaacccggcgatgtgcgagatcaccgaagacctgttctttaacgatccgtaccagattcacgaacgtaacaactatccggcggagctggaagcggatgtggcggcgctgcgtgatgatgcgcaactgaagctggcggttgcggcgctgaaacaccgtttctttgcgcatgcggaggcgctgctgcatggtgacattcacagcggcagcatcttcgttgcggagggtagcctgaaggcgatcgacgcggaattcggttactttggcccgatcggttttgatattggtaccgcgatcggcaacctgctgctgaactattgcggtctgccgggtcaactgggtattcgtgatgcggcggcggcgcgtgaacagcgtctgaacgatatccaccaactgtggaccaccttcgcggagcgttttcaagcgctggcggcggaaaagacccgtgacgcggcgctggcgtacccgggttatgcgagcgcgttcctgaagaaagtgtgggcggatgcggttggtttttgcggcagcgagctgattcgtcgtagcgtgggcctgagccacgttgcggacatcgataccattcaggacgatgcgatgcgtcacgaatgcctgcgtcacgcgatcaccctgggtcgtgcgctgattgttctggcggagcgtatcgacagcgtggatgaactgctggcgcgtgttcgtcaatacagctaa

SEQ ID NO: 3 - protein, 407 aa, Artificial Sequence
  Description of Artificial Sequence Synthetic polypeptide
  MTR kinase (rd2BB)
MSQYHTFTAHDAVAYAQQFAGIDNPSELVSAQEVGDGNLNLVFKVFDRQGVSRAIVKQALPYVRCVGESWPLTLDRARHEAQTLVAHYQHSPQHTVKIHHFDPELAVMVMEDLSDHRIWRGELIANVYYPQAARQLGDYLAQVLFHTSDFYLHPHEKKAQVAQFINPAMCEITEDLFFNDPYQIHERNNYPAELEADVAALRDDAQLKLAVAALKHRFFAHAEALLHGDIHSGSIFVAEGSLKAIDAEFGYFGPIGFDIGTAIGNLLLNYCGLPGQLGIRDAAAAREQRLNDIHQLWTTFAERFQALAAEKTRDAALAYPGYASAFLKKVWADAVGFCGSELIRRSVGLSHVADIDTIQDDAMRHECLRHAITLGRALIVLAERIDSVDELLARVRQYSLEHHHHHH

SEQ ID NO: 4 - DNA, 1224 bp, Artificial Sequence
  Description of Artificial Sequence Synthetic polynucleotide
  corresponding DNA seq 3
atgagccagtatcataccttcaccgcgcatgatgcggtggcgtatgcgcagcaatttgcgggcattgataacccgagcgagctggttagcgcgcaagaagttggtgacggcaacctgaacctggtgttcaaggtttttgatcgtcagggtgtgagccgtgcgatcgttaaacaagcgctgccgtacgtgcgttgcgttggtgaaagctggccgctgaccctggaccgtgcgcgtcatgaagcgcagaccctggtggcgcactatcagcacagcccgcaacacaccgttaagatccaccacttcgatccggagctggcggtgatggttatggaagacctgagcgatcaccgtatttggcgtggtgagctgatcgcgaacgtgtactatccgcaggcggcgcgtcaactgggtgactacctggcgcaggttctgttccacaccagcgatttttatctgcacccgcacgagaagaaagcgcaggtggcgcaattcattaacccggcgatgtgcgagatcaccgaagacctgttctttaacgatccgtaccagattcacgaacgtaacaactatccggcggagctggaagcggatgtggcggcgctgcgtgatgatgcgcaactgaagctggcggttgcggcgctgaaacaccgtttctttgcgcatgcggaggcgctgctgcatggtgacattcacagcggcagcatcttcgttgcggagggtagcctgaaggcgatcgacgcggaattcggttactttggcccgatcggttttgatattggtaccgcgatcggcaacctgctgctgaactattgcggtctgccgggtcaactgggtattcgtgatgcggcggcggcgcgtgaacagcgtctgaacgatatccaccaactgtggaccaccttcgcggagcgttttcaagcgctggcggcggaaaagacccgtgacgcggcgctggcgtacccgggttatgcgagcgcgttcctgaagaaagtgtgggcggatgcggttggtttttgcggcagcgagctgattcgtcgtagcgtgggcctgagccacgttgcggacatcgataccattcaggacgatgcgatgcgtcacgaatgcctgcgtcacgcgatcaccctgggtcgtgcgctgattgttctggcggagcgtatcgacagcgtggatgaactgctggcgcgtgttcgtcaatacagcctcgagcaccaccaccaccaccactga

SEQ ID NO: 5 - protein, 407 aa, Artificial Sequence
  Description of Artificial Sequence Synthetic polypeptide
  MTR kinase (rd3BB)
MSQYHTFTAHDAVAYAQQFAGIDNPSELVSAQEVGDGNLNLVFKVFDRQGVSRAIVKQALPYPRCVGESWPLTLDRARHEAQTLVAHYQHSPQHTVKIHHFDPELAVMVMEDLSDHRIWRGELIANVYYPQAARQLGDYLAQVLFHTSDFYLHPHEKKAQVAQFINPAMCEISEDLIFNDPYQIHERNNYPAELEADVAALRDDAQLKLAVAALKHRFFAHAEALLHGDLHSGSIFVAEGSLKAIDAEFGYFGPIGFDIGTAIGNLLLNYCGLPGQLGIRDAAAAREQRLNDIHQLWTTFAERFQALAAEKTRDAALAYPGYASAFLKKVWADAVGFCGSELIRRSVGLSHVADIDTIQDDAMRHECLRHAITLGRALIVLAERIDSVDELLARVRQYSLEHHHHHH

SEQ ID NO: 6 - DNA, 1224 bp, Artificial Sequence
  Description of Artificial Sequence Synthetic polynucleotide
  Corresponding DNA seq 5
atgagccagtatcataccttcaccgcgcatgatgcggtggcgtatgcgcagcaatttgcgggcattgataacccgagcgagctggttagcgcgcaagaagttggtgacggcaacctgaacctggtgttcaaggtttttgatcgtcagggtgtgagccgtgcgatcgttaaacaagcgctgccgtacccccgttgcgttggtgaaagctggccgctgaccctggaccgtgcgcgtcatgaagcgcagaccctggtggcgcactatcagcacagcccgcaacacaccgttaagatccaccacttcgatccggagctggcggtgatggttatggaagacctgagcgatcaccgtatttggcgtggtgagctgatcgcgaacgtgtactatccgcaggcggcgcgtcaactgggtgactacctggcgcaggttctgttccacaccagcgatttttatctgcacccgcacgagaagaaagcgcaggtggcgcaattcattaacccggcgatgtgcgagatcagcgaagacctgatttttaacgatccgtaccagattcacgaacgtaacaactatccggcggagctggaagcggatgtggcggcgctgcgtgatgatgcgcaactgaagctggcggttgcggcgctgaaacaccgtttctttgcgcatgcggaggcgctgctgcatggtgaccttcacagcggcagcatcttcgttgcggagggtagcctgaaggcgatcgacgcggaattcggttactttggcccgatcggttttgatattggtaccgcgatcggcaacctgctgctgaactattgcggtctgccgggtcaactgggtattcgtgatgcggcggcggcgcgtgaacagcgtctgaacgatatccaccaactgtggaccaccttcgcggagcgttttcaagcgctggcggcggaaaagacccgtgacgcggcgctggcgtacccgggttatgcgagcgcgttcctgaagaaagtgtgggcggatgcggttggtttttgcggcagcgagctgattcgtcgtagcgtgggcctgagccacgttgcggacatcgataccattcaggacgatgcgatgcgtcacgaatgcctgcgtcacgcgatcaccctgggtcgtgcgctgattgttctggcggagcgtatcgacagcgtggatgaactgctggcgcgtgttcgtcaatacagcctcgagcaccaccaccaccaccactga

SEQ ID NO: 7 - protein, 407 aa, Artificial Sequence
  Description of Artificial Sequence Synthetic polypeptide
  MTR kinase (rd4BB)
MSQYHTFTADDAVAYAQQFAGIDNPSELVSAQEVGDGNLNLVFKVFDRQGVSRAIVKQALPYPRRVGPSWPLTLDRARHEAQTLVAHYQHSPQHTVKIHHFDPELAVMVMEDLSDHRIWRGELIANVYYPQAARQLGDYLAQVLFHTSDFYLHPHEKKAQVAQFINPAMCEISEDLVFNDPYQIHERNNYPAELEADVAALRDDAQLKLAVAALKHRFFAHAEALLHGDLHTGSIFVAEGSLKVIDAEFGYFGPIGFDIGTAIGNLLLNYCGLPGQLGIRDAAAAREQRLNDIHQLWTTFAERFQALAAEKTRDAALAYPGYASAFLKKVWADAVGFCGSELIRRSVGLAHVADIDTIQDDAMRHECLREAITLGRALIVLAERIDSVDELLARVRQYSLEHHHHHH

SEQ ID NO: 8 - DNA, 1224 bp, Artificial Sequence
  Description of Artificial Sequence Synthetic polynucleotide
  Corresponding DNA seq 7
atgagccagtatcataccttcaccgcggatgatgcggtggcgtatgcgcagcaatttgcgggcattgataacccgagcgagctggttagcgcgcaagaagttggtgacggcaacctgaacctggtgttcaaggtttttgatcgtcagggtgtgagccgtgcgatcgttaaacaagcgctgccgtacccccgtcgcgttggtccgagctggccgctgaccctggaccgtgcccgtcatgaagcgcagaccctggttgcgcactatcagcacagcccgcaacacaccgttaagatccaccacttcgatccggagctggcggtgatggttatggaagacctgagcgatcaccgtatttggcgtggtgagctgatcgcgaacgtgtactatccgcaggcggcgcgtcaactgggtgactacctggcgcaggttctgttccacaccagcgatttttatctgcacccgcacgagaagaaagcgcaggtggcgcaattcattaacccggccatgtgcgaaatcagcgaagacctggtgtttaacgatccgtaccagattcacgaacgtaacaactatccggcggagctggaagcggatgtggcggcgctgcgtgatgatgcgcaactgaagctggcggttgcggcgctgaaacaccgtttctttgcgcatgcggaggcgctgctgcatggtgaccttcacaccggcagcatcttcgttgcggagggtagcctgaaggtgatcgacgcggaattcggttactttggcccgatcggttttgatattggtaccgcgatcggcaacctgctgctgaactattgcggtctgccgggtcaactgggtattcgtgatgcggcggcggcgcgtgaacagcgtctgaacgatatccaccaactgtggaccaccttcgcggagcgttttcaagcgctggcggcggaaaagacccgtgacgcggcgctggcgtacccgggttatgcgagcgcgttcctgaagaaagtgtgggcggatgcggttggtttttgcggcagcgagctgattcgtcgtagcgtgggcctggcgcacgttgcggacatcgataccattcaggacgatgcgatgcgtcacgaatgcctgcgtgaagcgatcaccctgggtcgtgcgctgattgttctggcggagcgcatcgacagcgtggatgaactgctggcgcgtgttcgtcaatacagcctcgagcaccaccaccaccaccactga

SEQ ID NO: 9 - protein, 407 aa, Artificial Sequence
  Description of Artificial Sequence Synthetic polypeptide
  MTR kinase F12
MSQYHTFTADDAVAYAQQFAGIDNPSELVSAQEVGDGNLNLVFKVFDRQGVSRAIVKQALPYPRAVGPSWPLTLDRARHEAQTLVAHYQHSPQHTVKIHHFDPELAVMVMEDLSDHRIWRGELIANVYYPQAARQLGDYLAQVLFHTSDFYLHPHEKKAQVAQFINPGMCEISEDLSFNDPYQIHERNNYPAELEADVAALRDDAQLKLAVAALKHRFFAHAEALLHGDLHSGSIFVAEGSLKVIDAEFGYFGPIGFDIGTAIGNLLLNYCGLPGQLGIRDAAAAREQRLNDIHQLWTTFAERFQALAAEKTRDAALAYPGYASAFLKKVWADAVGFCGSELIRRSVGLSHVADIDTIQDDAMRHECLRHAITLGRALIVLAETIDSVDELLARVRQYSLEHHHHHH

SEQ ID NO: 10 - DNA, 1224 bp, Artificial Sequence
  Description of Artificial Sequence Synthetic polynucleotide
  Corresponding DNA seq 8
atgagccagtatcataccttcaccgcggatgatgcggtggcgtatgcgcagcaatttgcgggcattgataacccgagcgagctggttagcgcgcaagaagttggtgacggcaacctgaacctggtgttcaaggtttttgatcgtcagggtgtgagccgtgcgatcgttaaacaagcgctgccgtacccccgtgcggttggtccgagctggccgctgaccctggaccgtgcgcgtcatgaagcgcagaccctggtggcgcactatcagcacagcccgcaacacaccgttaagatccaccacttcgatccggagctggcggtgatggttatggaagacctgagcgatcaccgtatttggcgtggtgagctgatcgcgaacgtgtactatccgcaggcggcgcgtcaactgggtgactacctggcgcaggttctgttccacaccagcgatttttatctgcacccgcacgagaagaaagcgcaggtggcgcaattcattaacccgggcatgtgcgaaatcagcgaagacctgagctttaacgatccgtaccagattcacgaacgtaacaactatccggcggagctggaagcggatgtggcggcgctgcgtgatgatgcgcaactgaagctggcggttgcggcgctgaaacaccgtttctttgcgcatgcggaggcgctgctgcatggtgaccttcacagcggcagcatcttcgttgcggagggtagcctgaaggtgatcgacgcggaattcggttactttggcccgatcggttttgatattggtaccgcgatcggcaacctgctgctgaactattgcggtctgccgggtcaactgggtattcgtgatgcggcggcggcgcgtgaacagcgtctgaacgatatccaccaactgtggaccaccttcgcggagcgttttcaagcgctggcggcggaaaagacccgtgacgcggcgctggcgtacccgggttatgcgagcgcgttcctgaagaaagtgtgggcggatgcggttggtttttgcggcagcgagctgattcgtcgtagcgtgggcctgtcgcacgttgcggacatcgataccattcaggacgatgcgatgcgtcacgaatgcctgcgtcacgcgatcaccctgggtcgtgcgctgattgttctggcggagaccatcgacagcgtggatgaactgctggcgcgtgttcgtcaatacagcctcgagcaccaccaccaccaccactga

SEQ ID NO: 11 - protein, 407 aa, Artificial Sequence
  Description of Artificial Sequence Synthetic polypeptide
  MTR kinase (rd5BB)
MSQYHTFTADDAVAYAQQFAGIDNPSELVSAQEVGDGNLNLVFKVFDRQGVSRAIVKQALPYPRRVGPSWPLTLDRARHEAQTLVAHYQHSPQHTVKIFHFDPELAVMVMEDLSDHRIWRGELIANVYYPQAARQLGDYLAQVLFHTSDFYLHPHEKKRQVAQFINPAMCGISEDLVFNDPYQIHERNNYPAELEAQVAALRDDAQLKLAVAALKHRFFAHAEALLHGDLHTGSIFVKEGSLKVIDAEFGYFGPIGFDIGTAIGNLLLNYCGLPGQLGIRDAAAAREQRLNDIHQLWTTFAERFQALAAEKTRDAALAYPGYASAFLKKVWADAVGFCGSELIRRSVGLAHVADIDTIQDDAMRHECLREAITLGRALIVLAERIDSVDELLARVRQYSLEHHHHHH

SEQ ID NO: 12 - protein, 407 aa, Artificial Sequence
  Description of Artificial Sequence Synthetic polypeptide
  MTR kinase (rd6BB)
MSQYHTFTADDAVAYAQQFAGIDNPSELVSAQEVGDGNLNLVFKVFDRQGVSRSIVKQALPYPRRVGPSWPLTLDRARHEAQTLVAHYQHSPQHTVKIFHFDPELAVMVMEDLSDHRIWRGELIANVYYPQAARQLGDYLAQVLFHTSDFYLHPHEKKRQVAQFINPAMCGISEDLVFNDPYQIHERNNYPAELEAQVAALRDDAQLKLAVAALKHRFFAHAEALLHGDLHTGSIFVKEGSLKVIDAEFGYFGPIGFDIGTAIGNLLLNYCGLPGQLGIRDAAAAREQRLNDIHQLWTTFAERFQALAAEKTRDAALRYPGYASAFLKKVWADAVGFCGSELIRRSVGLAHVADIDTIQDDAMRHECLREAITLGRALIVLAERIDSVDELLARVRQYSLEHHHHHH

SEQ ID NO: 13 - protein, 397 aa, Bacillus subtilis
MGVTKTPLYETLNESSAVALAVKLGLFPSKSTLTCQEIGDGNLNYVFHIYDQEHDRALIIKQAVPYAKVVGESWPLTIDRARIESSALIRQGEHVPHLVPRVFYSDTEMAVTVMEDLSHLKIARKGLIEGENYPHLSQHIGEFLGKTLFYSSDYALEPKVKKQLVKQFTNPELCDITERLVFTDPFFDHDTNDFEEELRPFVEKLWNNDSVKIEAAKLKKSFLTSAETLIHGDLHTGSIFASEHETKVIDPEFAFYGPIGFDVGQFIANLFLNALSRDGADREPLYEHVNQVWETFEETFSEAWQKDSLDVYANIDGYLTDTLSHIFEEAIGFAGCELIRRTIGLAHVADLDTIVPFDKRIGRKRLALETGTAFIEKRSEFKTITDVIELFKLLVKE

SEQ ID NO: 14 - protein, 403 aa, Thermotoga maritima
MRVLVINSGSSSIKYQLIEMEGEKVLCKGIAERIGIEGSRLVHRVGDEKHVIERELPDHEEALKLILNTLVDEKLGVIKDLKEIDAVGHRVVHGGERFKESVLVDEEVLKAIEEVSPLAPLHNPANLMGIKAAMKLLPGVPNVAVFDTAFHQTIPQKAYLYAIPYEYYEKYKIRRYGFHGTSHRYVSKRAAEILGKKLEELKIITCHIGNGASVAAVKYGKCVDTSMGFTPLEGLVMGTRSGDLDPAIPFFIMEKEGISPQEMYDILNKKSGVYGLSKGFSSDMRDIEEAALKGDEWCKLVLEIYDYRIAKYIGAYAAAMNGVDAIVFTAGVGENSPITREDVCSYLEFLGVKLDKQKNEETIRGKEGIISTPDSRVKVLVVPTNEELMIARDTKEIVEKIGR

SEQ ID NO: 15 - DNA, 1212 bp, Artificial Sequence
  Description of Artificial Sequence Synthetic polynucleotide
  Corresponding DNA seq  14
atgcgtgttctggttattaatagcggtagcagcagcattaaatatcagctgattgaaatggaaggtgaaaaagttctgtgtaaaggtattgcagaacgtattggtattgaaggtagccgtctggttcatcgtgttggtgatgaaaaacatgttattgaacgtgaactgccggatcatgaagaagcactgaaactgattctgaataccctggttgatgaaaaactgggtgttattaaagatctgaaagaaattgacgcagttggtcatcgtgttgttcatggtggtgaacgttttaaagaaagcgttctggttgatgaagaagttctgaaagcaattgaagaagttagcccgctggcaccgctgcataatccggcaaatctgatgggtattaaagcagcaatgaaactgctgccgggtgttccgaatgttgcagtttttgataccgcatttcatcagaccattccgcagaaagcatatctgtatgcaattccgtatgaatattacgaaaaatacaaaattcgtcgctacggttttcatggtaccagccatcgttatgttagcaaacgtgcagcagaaattctgggtaaaaaactggaagaactgaaaattatcacctgtcatattggtaatggtgcaagcgttgcagcagttaaatatggtaaatgtgttgataccagcatgggttttaccccgctggaaggtctggttatgggtacccgtagcggtgatctggatccggcaattccgttttttattatggaaaaagagggtattagcccgcaggaaatgtatgatattctgaataaaaaaagcggcgtttatggtctgagcaaaggttttagcagcgatatgcgtgatattgaagaagcagcactgaaaggtgatgaatggtgtaaactggttctggaaatttatgattaccgtattgcaaaatacatcggtgcatacgctgcagcaatgaatggtgttgatgcaattgtttttaccgcaggtgttggtgaaaatagcccgattacccgtgaagatgtttgtagctatctggaatttctgggtgttaaactggataaacagaaaaatgaagagaccattcgtggtaaagaaggtattattagcaccccggatagccgtgttaaagttctggttgttccgaccaatgaagaactgatgattgcacgtgataccaaagaaattgttgaaaaaatcggtcgttaa

SEQ ID NO: 16 - protein, 413 aa, Artificial Sequence
  Description of Artificial Sequence Synthetic polypeptide
  acetate kinase AcK-101
MGSHHHHHHGSRVLNINSGSSSIKYQLIEMEGEKVLCKGIAERIGIEGSRLVHRVGDEKHVIERELPDHEEALKLILNTLVDEKLGVIKDLKEIDAVGHRVVHGGERFKESVLVDEEVLKAIEEVSPLAPLHNPANLMGIKAAMKLLPGVPNVQVFDTAFHQTIPQKAYLYAIPYEYYEKYKIRRYGFHGISHRYVSKRAAEILGKKLEELKIITCHIGNGASVAAVKYGKCVDTSMGFTPLEGLVMGTRSGDLDPAIPFFIMEKEGISPQEMYDILNKKSGVYGLSKGFSSDMRDNFEAALKGDEWCKLVLEIYDYRIAKYIGAYAAAMNGVDAIVFTAGVGENSPITREDVCKYLEFLGVKLDKQKNEETILGKEGIISTPDSRVKVLVVPTNEELMIARDTKEIVEKIGR

SEQ ID NO: 17 - DNA, 1242 bp, Artificial Sequence
  Description of Artificial Sequence Synthetic polynucleotide
  Corresponding DNA seq  16
atgggtagccatcatcatcatcatcacggtagccgtgttctgaatatcaatagcggtagcagcagcattaaatatcagctgattgaaatggaaggtgaaaaagttctgtgtaaaggtattgcagaacgtattggtattgaaggtagccgtctggttcatcgtgttggtgatgaaaaacatgttattgaacgtgaactgccggatcatgaagaagcactgaaactgattctgaataccctggttgatgaaaaactgggtgttattaaagatctgaaagaaattgacgcagttggtcatcgtgttgttcatggtggtgaacgttttaaagaaagcgttctggttgatgaagaagttctgaaagcaattgaagaagttagcccgctggcaccgctgcataatccggcaaatctgatgggtattaaagcagcaatgaaactgctgccgggtgttccgaatgttcaagtttttgataccgcatttcatcagaccattccgcagaaagcatatctgtatgcaattccgtatgaatattacgaaaaatacaaaattcgtcgctacggttttcatggtatcagccatcgttatgttagcaaacgtgcagcagaaattctgggtaaaaaactggaagaactgaaaattatcacctgtcatattggtaatggtgcaagcgttgcagcagttaaatatggtaaatgtgttgataccagcatgggttttaccccgctggaaggtctggttatgggtacccgtagcggtgatctggatccggcaattccgttttttattatggaaaaagagggtattagcccgcaggaaatgtatgatattctgaataaaaaaagcggcgtttatggtctgagcaaaggttttagcagcgatatgcgtgataattttgaagcagcactgaaaggtgatgaatggtgtaaactggttctggaaatttatgactaccgtattgcaaaatacatcggtgcatacgctgcagcaatgaatggtgttgatgcaattgtttttaccgcaggtgttggtgaaaatagcccgatcacccgtgaagatgtttgtaagtatctggaatttctgggtgttaaactggataaacagaaaaatgaagagactattctgggtaaagaaggtattattagcaccccggatagccgtgttaaagttctggttgttccgaccaatgaagaactgatgattgcacgtgataccaaagaaattgttgaaaaaatcggtcgttaa

SEQ ID NO: 18 - protein, 592 aa, Aerococcus viridans
MSDNKINIGLAVMKILESWGADTIYGIPSGTLSSLMDAMGEEENNVKFLQVKHEEVGAMAAVMQSKFGGNLGVTVGSGGPGASHLINGLYDAAMDNIPVVAILGSRPQRELNMDAFQELNQNPMYDHIAVYNRRVAYAEQLPKLVDEAARMAIAKRGVAVLEVPGDFAKVEIDNDQWYSSANSLRKYEPIAPAAQDIDAAVELLNNSKRPVIYAGIGTMGHGPAVQELARKIKAPVITTGKNFETFEWDFEALTGSTYRVGWKPANETILEADTVLFAGSNFPFSEVEGTFRNVDNFIQIDIDPAMLGKRHHADVAILGDAGLAIDEILNKVDAVEESAWWTANLKNIANWREYINMLETKEEGDLQFYQVYNAINNHADEDAIYSIDVGNSTQTSIRHLHMTPKNMWRTSPLFATMGIAIPGGLGAKNTYPDRQVWNIIGDGAFSMTYPDVVTNVRYNMPVINVVFSNTEYAFIKNKYEDTNKNLFGVDFTDVDYAKIAEAQGAKGFTVSRIEDMDRVMAEAVAANKAGHTVVIDCKITQDRPIPVETLKLDSKLYSEDEIKAYKERYEAANLVPFREYLEAEGLESKYIK

SEQ ID NO: 19 - DNA, 1779 bp, Artificial Sequence
  Description of Artificial Sequence Synthetic polynucleotide
  Corresponding DNA seq 18
atgagtgataataaaatcaacatcggtctggcggtgatgaaaattctggaaagttggggcgcagataccatttatggcattccgagcggcaccctgagtagcttaatggatgccatgggtgaagaagaaaacaatgttaagttcctgcaggtgaagcatgaagaagtgggcgcaatggcagccgttatgcagagtaaatttggcggtaatctgggtgtgaccgttggtagtggcggtcctggtgcaagtcatctgattaatggcctgtatgatgccgcaatggataatattccggttgtggcaattctgggtagccgtcctcagcgtgaactgaatatggatgcatttcaggaactgaaccagaatccgatgtatgatcatatcgccgtttacaatcgccgtgtggcctatgcagaacagctgcctaaactggtggatgaagccgcccgtatggccattgctaaacgcggtgtggcagttctggaagttccgggtgattttgcaaaagtggaaattgataacgaccagtggtacagtagcgcaaatagcctgcgcaaatatgaaccgattgccccggcagcacaggatattgatgcagctgtggaactgctgaataatagtaaacgcccggtgatttacgcaggtattggtaccatgggccatggccctgcagttcaagaactggctcgtaaaattaaggcaccggtgattaccaccggtaaaaattttgaaaccttcgagtgggacttcgaagccctgaccggttcaacctatcgtgtgggttggaaaccggcaaatgaaaccattctggaagccgataccgttctgtttgcaggcagcaattttccgtttagcgaagtggaaggcacctttcgtaatgttgataatttcatccagatcgacatcgacccggcaatgctgggtaaacgccatcatgcagatgtggcaattctgggtgatgccggcctggcaattgatgaaattctgaataaggtggacgccgtggaagaaagcgcctggtggaccgcaaatctgaaaaatattgcaaactggcgcgagtatatcaacatgctggaaaccaaagaggagggtgatctgcagttttatcaggtttataacgcgatcaacaaccacgccgatgaagatgcaatttatagcattgacgtgggcaatagcacccagaccagcattcgtcatctgcacatgaccccgaaaaatatgtggcgcaccagcccgctgtttgccaccatgggtattgcaattccgggtggcctgggtgcaaaaaatacctatccggatcgtcaggtgtggaatattattggcgatggcgcctttagtatgacctatccggatgttgttaccaatgttcgctataatatgccggttatcaacgtggttttcagtaataccgagtacgcatttatcaagaacaagtacgaggacaccaacaaaaacctgtttggtgtggatttcaccgatgtggattatgccaaaatcgccgaagcacagggtgcaaaaggctttaccgtgagtcgcattgaagatatggatcgtgttatggccgaagccgtggccgcaaataaagccggtcataccgttgtgattgattgtaaaatcacccaggaccgtccgattccggttgaaaccctgaaactggatagcaaactgtatagtgaggacgaaatcaaggcatataaggaacgttacgaggcagcaaatctggtgccgtttcgtgaatatctggaagccgaaggtctggaaagcaaatatattaagtaa

SEQ ID NO: 20 - protein, 591 aa, Streptococcus sanguinis
MTQGKITASAAMLNVLKTWGVDTIYGIPSGTLSSLMDALAEDKDIRFLQVRHEETGALAAVMQAKFGGSIGVAVGSGGPGATHLINGVYDAAMDNTPFLAILGSRPVNELNMDAFQELNQNPMYHGIAVYNKRVAYAEQLPKVIDEACRAAVSKKGPAVVEIPVNFGFQEIDENSYYGSGSYERSFIAPALNEVEIDKAVEILNKAERPVIYAGFGGVKAGEVITELSRKIKAPIITTGKNFEAFEWNYEGLTGSAYRVGWKPANEVVFEADTVLFLGSNFPFAEVYEAFKNTEKFIQVDIDPYKLGKRHALDASILGDAGQAAKAILDKVDAVESTPWWRANVKNNQNWRDYMNKLEGKTEGELQLYQVYNAINKHADQDAIYSIDVGNSTQTSTRHLHMTPKNMWRTSPLFATMGIALPGGIAAKKDNPERQVWNIMGDGAFNMCYPDVITNVQYNLPVINVVFSNAEYAFIKNKYEDTNKHLFGVDFTNADYAKIAEAQGAVGFTVDRIEDIDAVVAEAVKLNKEGKTVVIDARITQHRPLPVEVLELDPKLHSEEAIKAFKEKYEAEELVPFRLFLEEEGLQSRAIK

SEQ ID NO: 21 - DNA, 1776 bp, Artificial Sequence
  Description of Artificial Sequence Synthetic polynucleotide
  Corresponding DNA seq  20
atgacccagggtaaaattaccgcaagtgccgcaatgctgaatgtgctgaaaacctggggcgttgataccatttatggtattccgagtggtaccctgagcagcctgatggatgccttagcagaagataaagatatccgctttctgcaggtgcgccatgaagaaaccggcgcattagccgcagtgatgcaggcaaaatttggtggcagcattggtgttgcagtgggtagcggtggtccgggtgcaacccacctgatcaatggtgtttatgatgccgccatggataataccccgtttctggccattctgggtagtcgccctgtgaatgaactgaatatggatgcattccaggagctgaatcagaatccgatgtatcatggtatcgccgtttataataagcgcgttgcatacgctgaacagctgccgaaagtgattgatgaagcatgccgtgcagccgtgagcaaaaaaggccctgcagtggttgaaattccggttaattttggcttccaggaaatcgatgagaacagttattacggcagcggtagttatgaacgtagctttattgccccggccctgaatgaagtggaaattgataaagcagtggagatcctgaacaaggcagaacgcccggtgatttatgcaggctttggtggcgtgaaagcaggtgaagtgattaccgaactgagccgcaaaattaaagccccgattattaccaccggcaaaaattttgaggcctttgaatggaactacgagggactgaccggcagtgcatatcgtgtgggttggaaaccggcaaatgaagtggtgtttgaagccgataccgtgctgtttctgggtagcaattttccgtttgccgaagtttatgaggcatttaaaaacaccgagaagttcatccaggtggatattgatccgtataagctgggcaaacgtcatgcactggatgcaagtattctgggtgatgcaggtcaggccgcaaaagcaattctggataaagttgatgccgtggaaagcaccccgtggtggcgtgcaaatgtgaaaaataatcagaactggcgcgactatatgaacaaactggaaggcaaaaccgagggtgaactgcagctgtatcaggtttataatgccattaacaagcacgcagaccaggatgcaatttatagtattgacgtgggcaacagcacccagaccagtacacgtcatctgcacatgaccccgaaaaatatgtggcgtaccagcccgctgtttgcaaccatgggtattgccctgccgggcggtattgctgcaaaaaaagataatccggagcgtcaggtttggaatattatgggtgatggtgcctttaacatgtgctatccggatgtgattaccaatgttcagtacaatctgccggttattaacgttgttttcagcaatgccgagtacgcatttattaagaacaagtacgaggacaccaacaagcatctgtttggtgttgatttcaccaacgccgattatgccaaaattgccgaagcacagggcgcagttggttttaccgtggatcgcattgaagatattgacgcagtggttgccgaagcagtgaaactgaataaagaaggtaagaccgtggtgattgacgcccgcattacccagcatcgccctttacctgtggaagtgctggaactggatccgaaactgcatagtgaagaagcaattaaggcctttaaggagaagtacgaagccgaagaactggtgccgtttcgtctgtttctggaagaagaaggcctgcagagtcgcgcaattaaataa

SEQ ID NO: 22 - protein, 606 aa, Weissella confusa
MSDKKISAGLAALKVMEGWGIDTMYGIPSGTLSGLMNAMGHPENKVKFIQVKHEEIGAMAAVMQYKFGGNIAVTVGSGGPGATHLINGLYDAYMDHIPVLAILGSKPVRELNMDSFQELNQNPMYDHIAVYNRRVATAEQLPHLVDDAIRTAISKRGVAVLEVPADFGFAELDAESIYSTPLYSSGTKYKRYKPVAPEAEEIDEAVEILKASERPVIYAGFGTQGHGDLVEELSRKIKAPVITTGKNFDNFNWDFEALTGSAFRVGWKPANEAVLEADTVLFIGTNFPFSEVEGTFRNVKKFIQIDANPDMLGKRHETDVAILADAGESLKALLAKVEPVADTPWWQANIKNVQNWRDYMNKLEQKTEGPLQAYQVYNAINKLADEDAIFSTDVGDVTQLSTRHLHMNPKQMWRTSPLFATMGIALPGGIGAKNIYPDRQVWNIIGDGAFSMTYPDVVTSVRYDMPMINVIFTNTEYGFIKNKYEDTNEYNFGVDFTDVDYAKVGEAQGAIGLTVNRIEDIDRVMQEAVDYYKQGRVVVVDAKITKDRPIPVETLKLDTNLYSEDVVKAYKEKYEAEALVPFREFLEGEGLKSIYIKEDNDNKFSF

SEQ ID NO: 23 - DNA, 1821 bp, Artificial Sequence
  Description of Artificial Sequence Synthetic polynucleotide
  Corresponding DNA seq  22
atgagtgataaaaaaatcagcgcgggcctggcagcactgaaagttatggaaggttggggtattgataccatgtatggtattccgagcggtaccctgagcggtttaatgaatgcaatgggccatccggaaaataaagttaaattcatccaggtgaagcacgaggaaattggcgcaatggcagccgttatgcagtataaatttggtggcaatatcgcggttaccgttggtagcggtggccctggtgcaacccacctgatcaatggtctgtatgatgcctatatggaccatattccggttctggcaattctgggtagtaaaccggttcgcgaactgaatatggatagttttcaggaactgaaccagaacccgatgtatgatcatattgccgtttacaaccgccgcgttgcaaccgcagaacagttacctcatctggtggatgatgcaattcgcaccgccattagtaaacgcggtgtggctgttctggaagttccggcagattttggttttgccgaactggatgccgaaagcatttatagcaccccgctgtatagtagtggcaccaaatataaacgctacaagccggttgcaccggaagccgaggaaattgatgaagccgtggaaattctgaaggcaagcgaacgtccggttatttatgccggttttggtacccagggccatggtgatctggtggaagaactgagtcgcaaaattaaagcaccggtgattaccaccggtaaaaattttgataacttcaactgggacttcgaggcactgaccggctcagcatttcgcgttggttggaaaccggccaatgaagccgtgctggaagccgacacagttctgtttattggcaccaatttcccgtttagtgaggtggaaggcacctttcgcaatgttaaaaaattcatccagatcgacgcaaacccggatatgctgggtaaacgtcatgaaaccgatgttgccattctggcagatgccggtgaaagcctgaaagccctgctggcaaaagttgaaccggtggcagataccccgtggtggcaggcaaatattaaaaatgtgcagaactggcgcgactatatgaataaactggaacagaagaccgagggcccgctgcaggcataccaggtgtacaatgcaattaataagctggccgatgaggatgccatttttagtaccgatgtgggtgatgttacccagctgagcacccgtcatctgcacatgaatccgaaacagatgtggcgtaccagtccgctgtttgcaaccatgggcattgccctgccgggtggtattggagcaaaaaatatttacccggaccgtcaggtttggaatattattggcgatggcgcatttagcatgacctatccggatgttgttaccagtgttcgttatgatatgccgatgattaacgtgatcttcaccaataccgagtacggttttatcaagaacaagtacgaggacaccaacgaatataacttcggcgtggattttaccgatgtggattatgccaaagtgggcgaagcccagggcgcaatcggtctgaccgtgaacagaattgaagatattgaccgcgtgatgcaggaagccgtggattattataaacagggtcgcgttgttgtggttgatgcaaaaattaccaaggaccgcccgattccggttgaaaccctgaaactggataccaatctgtatagtgaggacgttgttaaggcctataaggaaaaatacgaggcagaagcactggttccgtttcgcgaatttctggaaggtgaaggtctgaaaagtatctatatcaaggaggacaacgacaacaagttcagtttttaa

SEQ ID NO: 24 - protein, 593 aa, Streptococcus thermophilus
MTVGKTKVSTASLKVLAGWGIDTIYGIPSGTLAPLMEALGEQEETDIKFLQVKHEEVGAMAAVMQWKFGGKLGVCVGSGGPGASHLINGLYDAAMDNTPVLAILGSPPQRELNMDAFQELNQNPMYDHIAVYNRRVAYAEQLPKLIDDAIRTAISKRGVAVLEVPGDFGYKEIANDAFYSSGHSYRDYVSSAINEVDIDAAVEVLNKSKRAVIYAGIGTMGHGPAVQELSRKIKAPIITTAKNFETFDYDFEGLTGSTYRVGWKPANEAVKEADTVLFVGSNFPFAEVEGTFSNVENFIQIDNNPTMLGKRHNADVAILGDAGEAVQMLLEKVAPVEESAWWNANLKNIQNWRDYMTKLETKENGPLQLYQVYNAINKYADEDAIYSIDVGNTTQTSIRHLHMTPKNMWRTSPLFASMGIALPGGIGAKNVYPERQVFNLMGDGAFSMNYQDIVTNVRYNMPVINVVFTNTEYGFIKNKYEDTNTNTFGTEFTDVDYAMIGEAQGAVGFTVSRIEDMDQVMAAAVKANKEGKTVVIDAKITKDRPIPVETLKLDPALYSEEEIKAYKERYEAEELVPFSEFLKAEGLESKVAK

SEQ ID NO: 25 - protein, 253 aa, Escherichia coli
MSKSDVFHLGLTKNDLQGATLAIVPGDPDRVEKIAALMDKPVKLASHREFTTWRAELDGKPVIVCSTGIGGPSTSIAVEELAQLGIRTFLRIGTTGAIQPHINVGDVLVTTASVRLDGASLHFAPLEFPAVADFECTTALVEAAKSIGATTHVGVTASSDTFYPGQERYDTYSGRVVRHFKGSMEEWQAMGVMNYEMESATLLTMCASQGLRAGMVAGVIVNRTQQEIPNAETMKQTESHAVKIVVEAARRLL

SEQ ID NO: 26 - DNA, 762 bp, Escherichia coli
atgtccaagtctgatgtttttcatctcggcctcactaaaaacgatttacaaggggctacgcttgccatcgtccctggcgacccggatcgtgtggaaaagatcgccgcgctgatggataagccggttaagctggcatctcaccgcgaattcactacctggcgtgcagagctggatggtaaacctgttatcgtctgctctaccggtatcggcggcccgtctacctctattgctgttgaagagctggcacagctgggcattcgcaccttcctgcgtatcggtacaacgggcgctattcagccgcatattaatgtgggtgatgtcctggttaccacggcgtctgtccgtctggatggcgcgagcctgcacttcgcaccgctggaattcccggctgtcgctgatttcgaatgtacgactgcgctggttgaagctgcgaaatccattggcgcgacaactcacgttggcgtgacagcttcttctgataccttctacccaggtcaggaacgttacgatacttactctggtcgcgtagttcgtcactttaaaggttctatggaagagtggcaggcgatgggcgtaatgaactatgaaatggaatctgcaaccctgctgaccatgtgcgcaagtcagggcctgcgtgccggtatggtagcgggtgttatcgttaaccgcacccagcaagagatcccgaatgctgagacgatgaaacaaaccgaaagccatgcggtgaaaatcgtggtggaagcggcgcgtcgtctgctgtaa

SEQ ID NO: 27 - protein, 264 aa, Escherichia coli
MHHHHHHGGSGMSKSDVFHLGLTKNDLQGATLAIVPGDPDRVEKIAALMDKPVKLASHREFTTWRAELDGKPVIVCSTGIGGPSTSIAVEELAQLGIRTFLRIGTTGAIQPHINVGDVLVTTASVRLDGASLHFAPLEFPAVADFECTTALVEAAKSIGATTHVGVTASSDTFYPGQERYDTYSGRVVRHFKGSMEEWQAMGVMNYEMESATLLTMCASQGLRAGMVAGVIVNRTQQEIPNAETMKQTESHAVKIVVEAARRLL

SEQ ID NO: 28 - protein, 264 aa, Artificial Sequence
  Description of Artificial Sequence Synthetic polypeptide
  uridine phosphorylase
MHHHHHHGGSGMSKSDVFHLGLTKNDLQGATLAIVPGDPDRVEKIAALMDKPVKLASHREFTTWRAELDGKPVIVCSTGIGGPSTSIAVEILAQLGIRTFLRIGTTGAIQPHINVGDVLVTTASVRLDGASLHFAPLEFPAVADFECTTALVEAAKSIGATTHVGVTASSDTFYPGQERYDTYSGRVVRHFKGSMEEWQAMGVMNYEMESATLLTMCASQGLRAGMVAGVIVNRTQQEIPNAETMKQTESHAVKIVVEAARRLL

SEQ ID NO: 29 - DNA, 795 bp, Artificial Sequence
  Description of Artificial Sequence Synthetic polynucleotide
  Corresponding DNA seq  28
atgcaccatcatcatcatcatggcggtagcggcatgtccaagtctgatgtttttcatctcggcctcactaaaaacgatttacaaggggctacgcttgccatcgtccctggcgacccggatcgtgtggaaaagatcgccgcgctgatggataagccggttaagctggcatctcaccgcgaattcactacctggcgtgcagagctggatggtaaacctgttatcgtctgctctaccggtatcggcggcccgtctacctctattgctgttgaaattctggcacagctgggcattcgcaccttcctgcgtatcggtacaacgggcgctattcagccgcatattaatgtgggtgatgtcctggttaccacggcgtctgtccgtctggatggcgcgagcctgcacttcgcaccgctggaattcccggctgtcgctgatttcgaatgtacgactgcgctggttgaagctgcgaaatccattggcgcgacaactcacgttggcgtgacagcttcttctgataccttctacccaggtcaggaacgttacgatacttactctggtcgcgtagttcgtcactttaaaggttctatggaagagtggcaggcgatgggcgtaatgaactatgaaatggaatctgcaaccctgctgaccatgtgcgcaagtcagggcctgcgtgccggtatggtagcgggtgttatcgttaaccgcacccagcaagagatcccgaatgctgagacgatgaaacaaaccgaaagccatgcggtgaaaatcgtggtggaagcggcgcgtcgtctgctgtaa

SEQ ID NO: 30 - protein, 264 aa, Artificial Sequence
  Description of Artificial Sequence Synthetic polypeptide
  uridine phosphorylase
MHHHHHHGGSGMSESDSFHLGLTKNDLQGATLAILPGDPDRVEKIAALMDKPVKLASWREFTTWRAELDGKPVIVCSTGIGGPSTSIAVEILAQLGIRTFLRIGTTGAIQPHINVGDVLVTTASVRLDGASLHFAPLEFPAVADFECTTALVEAAKSIGATTHVGVTASSDTFYPGQERYDTYSGRVVRHFKGSMEEWQRMGVMNYEMESATLLTMCASQGLRAGMVAGVIVNRTQQELPNAESMKQTESHMVKIVVEAARRLL

SEQ ID NO: 31 - protein, 264 aa, Artificial Sequence
  Description of Artificial Sequence Synthetic polypeptide
  uridine phosphorylase
MHHHHHHGGSGMSESDSFHMGLTKNDLQGATLAILPGDPARVEKIAALMDNPVKLASWREFTTWRAELDGKPVIVCSTGIGGPSTSIAVEILAQLGIRTFLRIGTTGAIQGHINVGDVLVTTASVRLDGASGHFAPMEFPAVADFECTTALVEAAKSIGATTHVGVTASSDTFYPGQERYDTYSGRVVRRFQGSMEEWQEMGVMNYEMESATLLTMCASQGLRAGMVAGVIVNRTQQELPNAESMKQVESHMVKIVVEAARRLL

SEQ ID NO: 32 - protein, 309 aa, Escherichia coli
MQNAGSLVVLGSINADHILNLQSFPTPGETVTGNHYQVAFGGKGANQAVAAGRSGANIAFIACTGDDSIGESVRQQLATDNIDITPVSVIKGESTGVALIFVNGEGENVIGIHAGANAALSPALVEAQRERIANASALLMQLESPLESVMAAAKIAHQNKTIVALNPAPARELPDELLALVDIITPNETEAEKLTGIRVENDEDAAKAAQVLHEKGIRTVLITLGSRGVWASVNGEGQRVPGFRVQAVDTIAAGDTFNGALITALLEEKPLPEAIRFAHAAAAIAVTRKGAQPSVPWREEIDAFLDRQR

SEQ ID NO: 33 - DNA, 930 bp, Escherichia coli
atgcaaaacgcaggcagcctcgttgttcttggcagcattaatgctgaccacattcttaatcttcaatcttttcctactccaggcgaaaccgtaaccggtaaccactatcaggttgcatttggcggcaaaggcgcgaatcaggctgtggctgctgggcgtagcggtgcgaatatcgcgtttattgcctgtacgggtgatgacagcattggtgagagcgttcgccagcagctcgccactgataacattgatattactccggtcagcgtgatcaaaggcgaatcaacaggtgtggcgctgatttttgttaatggcgaaggtgagaatgtcatcggtattcatgccggcgctaatgctgccctttccccggcgctggtggaagcgcaacgtgagcgtattgccaacgcgtcagcattattaatgcagctggaatcaccactcgaaagtgtgatggcagcggcgaaaatcgcccatcaaaataagactatcgttgcgcttaacccggctccggctcgcgaacttcctgacgaactgctggcgctggtggacattattacgccaaacgaaacggaagcagaaaagctcaccggtattcgtgttgaaaatgatgaagatgcagcgaaggcggcgcaggtactgcatgaaaaaggtatccgtactgtactgattactttaggaagtcgtggtgtatgggctagcgtgaatggtgaaggtcagcgcgttcctggattccgggtgcaggctgtcgataccattgctgccggagatacctttaacggtgcgttaatcacggcattgctggaagaaaaaccattgccagaggcgattcgttttgcccatgctgccgctgcgattgccgtaacacgtaaaggcgcacaaccttccgtaccgtggcgtgaagagatcgacgcatttttagacaggcagaggtaa

SEQ ID NO: 34 - protein, 407 aa, Escherichia coli
MKRAFIMVLDSFGIGATEDAERFGDVGADTLGHIAEACAKGEADNGRKGPLNLPNLTRLGLAKAHEGSTGFIPAGMDGNAEVIGAYAWAHEMSSGKDTPSGHWEIAGVPVLFEWGYFSDHENSFPQELLDKLVERANLPGYLGNCHSSGTVILDQLGEEHMKTGKPIFYTSADSVFQIACHEETFGLDKLYELCEIAREELTNGGYNIGRVIARPFIGDKAGNFQRTGNRHDLAVEPPAPTVLQKLVDEKHGQVVSVGKIADIYANCGITKKVKATGLDALFDATIKEMKEAGDNTIVFTNFVDFDSSWGHRRDVAGYAAGLELFDRRLPELMSLLRDDDILILTADHGCDPTWTGTDHTREHIPVLVYGPKVKPGSLGHRETFADIGQTLAKYFGTSDMEYGKAMF

SEQ ID NO: 35 - DNA, 1224 bp, Escherichia coli
atgaaacgtgcatttattatggtgctggactcattcggcatcggcgctacagaagatgcagaacgctttggtgacgtcggggctgacaccctgggtcatatcgcagaagcttgtgccaaaggcgaagctgataacggtcgtaaaggcccgctcaatctgccaaatctgacccgtctggggctggcgaaagcacacgaaggttctaccggtttcattccggcgggaatggacggcaacgctgaagttatcggcgcgtacgcatgggcgcacgaaatgtcatccggtaaagataccccgtctggtcactgggaaattgccggtgtcccggttctgtttgagtggggatatttctccgatcacgaaaacagcttcccgcaagagctgctggataaactggtcgaacgcgctaatctgccgggttacctcggtaactgccactcttccggtacggtcattctggatcaactgggcgaagagcacatgaaaaccggcaagccgattttctatacctccgctgactccgtgttccagattgcctgccatgaagaaactttcggtctggataaactctacgaactgtgcgaaatcgcccgtgaagagctgaccaacggcggctacaatatcggtcgtgttatcgctcgtccgtttatcggcgacaaagccggtaacttccagcgtaccggtaaccgtcacgacctggctgttgagccgccagcaccgaccgtgctgcagaaactggttgatgaaaaacacggccaggtggtttctgtcggtaaaattgcggacatctacgccaactgcggtatcaccaaaaaagtgaaagcgactggcctggacgcgctgtttgacgccaccatcaaagagatgaaagaagcgggtgataacaccatcgtcttcaccaacttcgttgacttcgactcttcctggggccaccgtcgcgacgtcgccggttatgccgcgggtctggaactgttcgaccgccgtctgccggagctgatgtctctgctgcgcgatgacgacatcctgatcctcaccgctgaccacggttgcgatccgacctggaccggtactgaccacacgcgtgaacacattccggtactggtatatggcccgaaagtaaaaccgggctcactgggtcatcgtgaaaccttcgcggatatcggccagactctggcaaaatattttggtacttctgatatggaatatggcaaagccatgttctaa

SEQ ID NO: 36 - protein, 413 aa, Artificial Sequence
  Description of Artificial Sequence Synthetic polypeptide
  phosphopentomutase
MKRAFIMVLDSFGIGATEDAERFGDVGADTLGHIAEACAKGEADNGRKGPLNLPNLTRLGLAKAHEGSTGFIPAGMDGNAEVIGAYAWAHEMSSGKDTPSGHWEIAGVPVLFEWGYFSDHENSFPQELLDKLVERANLPGYLGNCHSSGTVILDQLGEEHMKTGKPIFYTSADSVFQIACHEETFGLDKLYELCEIAREELTNGGYNIGRVIARPFIGDKAGNFQRTGNRRDLAVEPPAPTVLQKLVDEKHGQVVSVGKIADIYANCGITKKVKATGLDALFDATIKEMKEAGDNTIVFTNFVDFDSSWGHRRDVAGYAAGLELFDRRLPELMSLLRDDDILILTADHGCDPTWTGTDHTREHIPVLVYGPKVKPGSLGHRETFADIGQTLAKYFGTSDMEYGKAMFHHHHHH

SEQ ID NO: 37 - DNA, 1242 bp, Artificial Sequence
  Description of Artificial Sequence Synthetic polynucleotide
  Corresponding DNA seq  36
atgaaacgtgcatttattatggtgctggactcattcggcatcggcgctacagaagatgcagaacgctttggtgacgtcggggctgacaccctgggtcatatcgcagaagcttgtgccaaaggcgaagctgataacggtcgtaaaggcccgctcaatctgccaaatctgacccgtctggggctggcgaaagcacacgaaggttctaccggtttcattccggcgggaatggacggcaacgctgaagttatcggcgcgtacgcatgggcgcacgaaatgtcatccggtaaagataccccgtctggtcactgggaaattgccggtgtcccggttctgtttgagtggggatatttctccgatcacgaaaacagcttcccgcaagagctgctggataaactggtcgaacgcgctaatctgccgggttacctcggtaactgccactcttccggtacggtcattctggatcaactgggcgaagagcacatgaaaaccggcaagccgattttctatacctccgctgactccgtgttccagattgcctgccatgaagaaactttcggtctggataaactctacgaactgtgcgaaatcgcccgtgaagagctgaccaacggcggctacaatatcggtcgtgttatcgctcgtccgtttatcggcgacaaagccggtaacttccagcgtaccggtaaccgtcgtgacctggctgttgagccgccagcaccgaccgtgctgcagaaactggttgatgaaaaacacggccaggtggtttctgtcggtaaaattgcggacatctacgccaactgcggtatcaccaaaaaagtgaaagcgactggcctggacgcgctgtttgacgccaccatcaaagagatgaaagaagcgggtgataacaccatcgtcttcaccaacttcgttgacttcgactcttcctggggccaccgtcgcgacgtcgccggttatgccgcgggtctggaactgttcgaccgccgtctgccggagctgatgtctctgctgcgcgatgacgacatcctgatcctcaccgctgaccacggttgcgatccgacctggaccggtactgaccacacgcgtgaacacattccggtactggtatatggcccgaaagtaaaaccgggctcactgggtcatcgtgaaaccttcgcggatatcggccagactctggcaaaatattttggtacttctgatatggaatatggcaaagccatgttccatcatcatcaccatcattaa

SEQ ID NO: 38 - protein, 413 aa, Artificial Sequence
  Description of Artificial Sequence Synthetic polypeptide
  phosphopentomutase
MKRAFIMVLDSFGIGATEDAEKFGDVGADTLGHIAEACAKGEADNGRKGPLNVPNLTRLGLAKAHEGSTGFIPAGMDGNAEVIGAYAWAHEMSSGKDTPSGHWEIAGVPVLFEWGYFSDLENSFPQELLDKLVERANLPGYLGNCHSSGTVILDQLGEEHMKTGKPIFYTSADSVFQIACHEETFGLDKLYELCEIAREELTNGGYNIGRVIARPFIGDKAGNFQRTGNRRDLAVEPPAPTVLQKLVDEKHGQVVGVGKIADIYANCGITKKVKATGLDALFDTTIKEMKEAGDNTIVFTNFVDFDSSWGHRRDVAGYAAGLELFDRRLPELMSLLRDDDILILTADHGCDPTWTGTDHTREHIPVLVYGPKVKPGSLGHRETFADIGQTLAKYFGTSDMEYGKAMFHHHHHH

SEQ ID NO: 39 - DNA, 1242 bp, Artificial Sequence
  Description of Artificial Sequence Synthetic polynucleotide
  Corresponding DNA seq 38
atgaaacgtgcatttattatggtgctggactcattcggcatcggcgctacagaagatgcagaaaagtttggtgacgtcggggctgacaccctgggtcatatcgcagaagcttgtgccaaaggcgaagctgataacggtcgtaaaggcccgctcaatgtgccaaatctgacccgtctggggctggcgaaagcgcacgaaggctctaccggtttcattccggcgggaatggacggcaacgctgaagttatcggcgcgtacgcatgggcgcacgaaatgtcatccggtaaagataccccgtctggtcactgggaaattgccggtgtcccggttctgttcgagtggggatatttctccgatctggaaaacagcttcccgcaagagctgctggataaactggtcgaacgcgctaatctgccgggttacctcggtaactgccactcttccggtacggtcattctggatcaactgggcgaagagcacatgaaaaccggcaagccgattttctatacctccgctgactccgtgttccagattgcctgccatgaagaaactttcggtctggataaactctacgaactgtgcgaaatcgcccgtgaagagctgaccaacggcggctacaatatcggtcgtgttatcgctcgtccgtttatcggcgacaaagccggtaacttccagcgtaccggtaaccgtcgtgacctggctgttgagccgccagcaccgaccgtgctgcagaaactggttgatgaaaaacacggccaggtggtaggcgtcggtaaaattgcggacatctacgccaactgcggtatcaccaaaaaagtgaaagcgactggcctggacgcgctgtttgacactaccatcaaagagatgaaagaagcgggtgataacaccatcgtcttcaccaacttcgttgacttcgactcttcctggggccaccgtcgcgacgtcgccggttatgccgcgggtctggaactgttcgaccgccgtctgccggagctgatgtctctgctgcgcgatgacgacatcctgatcctcaccgctgaccacggttgcgatccgacctggaccggtactgaccacacgcgtgaacacattccggtactggtatatggcccgaaagtaaaaccgggctcactgggtcatcgtgaaaccttcgcggatatcggccagactctggcaaaatattttggtacttctgatatggaatatggcaaagccatgttccatcatcatcaccatcattaa

SEQ ID NO: 40 - protein, 500 aa, Alloscardovia omnicolens
MKNKVQLITYADRLGDGTLKSMTETLRKHFEGVYEGVHILPFFTPFDGADAGFDPVDHTKVDPRLGSWDDVAELSTTHDIMVDTIVNHMSWESEQFQDVMAKGEDSEYYPMFLTMSSIFPDGVTEEDLTAIYRPRPGLPFTHYNWGGKTRLVWTTFTPQQVDIDTDSEMGWNYLLSILDQLSQSHVSQIRLDAVGYGAKEKNSSCFMTPKTFKLIERIKAEGEKRGLETLIEVHSYYKKQVEIASKVDRVYDFAIPGLLLHALEFGKTDALAQWIDVRPNNAVNVLDTHDGIGVIDIGSDQMDRSLAGLVPDEEVDALVESIHRNSKGESQEATGAAASNLDLYQVNCTYYAALGSDDQKYIAARAVQFFMPGVPQVYYVGALAGSNDMDLLKRTNVGRDINRHYYSAAEVASEVERPVVQALNALGRFRNTLSAFDGEFSYSNADGVLTMTWADDATRATLTFAPKANSNGASVARLEWTDAAGEHATDDLIANPPVVA

SEQ ID NO: 41 - protein, 500 aa, Artificial Sequence
  Description of Artificial Sequence Synthetic polypeptide
  sucrose phosphorylase SUP-101
MKNKVQLITYADRLGDGTLKSMTETLRKHFEGVYEGVHILPFFTPFDGADAGFDPVDHTKVDPRLGSWDDVAELSTTHDIMVDTIVNHMSWESEQFQDVMAKGEDSEYYPMFLTMSSIFPDGVTEEDLTAIYRPRPGLPFTHYNWGGKTRLVWTTFTRQQVDIDTDSEMGWNYLLSILDQLSQSHVSQIRLDAVGYGAKEKNSSCFLTPKTFKLVERIKAEGEKRGLETLIEVHSYYKKQVEIASKVDRVYDFAIPGLLLHALEFGKTDALAQWIDVRPNNAVNVLDTHDGIGVIDIGSDQMDRSLAGLVPDEEVDALVESIHRNSKGESQEATGAAASNLDLYQVNCTYYAALGSDDQKYIAARAVQFFMPGVPQVYYVGALAGSNDMDLLKRTNSGRGINRHYYSAAEVASEVERPVVQALNALGRFRNTLSAFDGEFSYSNADGVLTMTWADDATRATLTFAPKANSNGASVARLEWTDAAGEHATDDLIANPPVVA

SEQ ID NO: 42 - DNA, 1503 bp, Artificial Sequence
  Description of Artificial Sequence Synthetic polynucleotide
  Corresponding DNA seq 40
atgaagaacaaagttcaactgattacctatgcggatcgtctgggtgacggcaccctgaagagcatgaccgagaccctgcgtaaacacttcgagggtgtttatgaaggcgtgcacatcctgccgttctttaccccgttcgatggtgcggacgcgggctttgatccggttgaccacaccaaagtggacccgcgtctgggtagctgggacgatgtggcggaactgagcaccacccacgatatcatggttgacaccattgtgaaccacatgagctgggagagcgaacagttccaagatgttatggcgaagggcgaggacagcgaatactatccgatgttcctgaccatgagcagcatttttccggatggtgtgaccgaggaagacctgaccgcgatctatcgtccgcgtccgggtctgccgttcacccactataactggggtggcaaaacccgtctggtttggaccacctttacccgtcagcaagtggacattgataccgacagcgagatgggttggaactacctgctgagcatcctggatcagctgagccaaagccacgttagccaaattcgtctggacgcggtgggttatggcgcgaaggagaaaaacagcagctgcttcctgaccccgaagacctttaaactggtcgaacgtattaaggcggagggtgaaaaacgtggcctggagaccctgatcgaagttcacagctactataagaaacaggtggagattgcgagcaaggtggatcgtgtttacgactttgcgatcccgggtctgctgctgcatgcgctggaatttggcaaaaccgatgcgctggcgcaatggattgacgttcgtccgaacaacgcggtgaacgttctggatacccacgacggtatcggcgttatcgatattggtagcgatcagatggaccgtagcctggcgggtctggtgccggatgaggaagttgacgcgctggttgagagcatccaccgtaacagcaagggtgaaagccaggaagcgaccggcgcggcggcgagcaacctggacctgtaccaagttaactgcacctactatgcggcgctgggtagcgacgatcagaaatatattgcggcgcgtgcggtgcagttctttatgccgggcgtgccgcaagtttactatgtgggtgcgctggcgggcagcaacgatatggacctgctgaagcgtaccaacagtggtcgtggcatcaaccgtcactactatagcgcggcggaagtggcgagcgaggtggaacgtccggtggttcaggcgctgaacgcgctgggccgtttccgtaacaccctgagcgcgttcgatggtgaatttagctacagcaacgcggacggcgttctgaccatgacctgggcggatgatgcgacccgtgcgaccctgacctttgcgccgaaggcgaacagcaacggtgcgagcgtggcgcgtctggagtggaccgatgcggcgggtgaacatgcgaccgacgatctgatcgcgaacccgccggtggttgcgtaa

SEQ ID NO: 43 - protein, 510 aa, Artificial Sequence
  Description of Artificial Sequence Synthetic polypeptide
  sucrose phosphorylase SUP-101
MKNKVQLITYADRLGDGTLKSMTETLRKHFEGVYEGVHILPFFTPFDGADAGFDPVDHTKVDPRLGSWDDVAELSTTHDIMVDTIVNHMSWESEQFQDVMAKGEDSEYYPMFLTMSSIFPDGVTEEDLTAIYRPRPGLPFTHYNWGGKTRLVWTTFTRQQVDIDTDSEMGWNYLLSILDQLSQSHVSQIRLDAVGYGAKEKNSSCFLTPKTFKLVERIKAEGEKRGLETLIEVHSYYKKQVEIASKVDRVYDFAIPGLLLHALEFGKTDALAQWIDVRPNNAVNVLDTHDGIGVIDIGSDQMDRSLAGLVPDEEVDALVESIHRNSKGESQEATGAAASNLDLYQVNCTYYAALGSDDQKYIAARAVQFFMPGVPQVYYVGALAGSNDMDLLKRTNSGRGINRHYYSAAEVASEVERPVVQALNALGRFRNTLSAFDGEFSYSNADGVLTMTWADDATRATLTFAPKANSNGASVARLEWTDAAGEHATDDLIANPPVVAGQTGHHHHHH

SEQ ID NO: 44 - DNA, 1536 bp, Artificial Sequence
  Description of Artificial Sequence Synthetic polynucleotide
  Corresponding DNA seq  43
atgaagaacaaagttcaactgattacctatgcggatcgtctgggtgacggcaccctgaagagcatgaccgagaccctgcgtaaacacttcgagggtgtttatgaaggcgtgcacatcctgccgttctttaccccgttcgatggtgcggacgcgggctttgatccggttgaccacaccaaagtggacccgcgtctgggtagctgggacgatgtggcggaactgagcaccacccacgatatcatggttgacaccattgtgaaccacatgagctgggagagcgaacagttccaagatgttatggcgaagggcgaggacagcgaatactatccgatgttcctgaccatgagcagcatttttccggatggtgtgaccgaggaagacctgaccgcgatctatcgtccgcgtccgggtctgccgttcacccactataactggggtggcaaaacccgtctggtttggaccacctttacccgtcagcaagtggacattgataccgacagcgagatgggttggaactacctgctgagcatcctggatcagctgagccaaagccacgttagccaaattcgtctggacgcggtgggttatggcgcgaaggagaaaaacagcagctgcttcctgaccccgaagacctttaaactggtcgaacgtattaaggcggagggtgaaaaacgtggcctggagaccctgatcgaagttcacagctactataagaaacaggtggagattgcgagcaaggtggatcgtgtttacgactttgcgatcccgggtctgctgctgcatgcgctggaatttggcaaaaccgatgcgctggcgcaatggattgacgttcgtccgaacaacgcggtgaacgttctggatacccacgacggtatcggcgttatcgatattggtagcgatcagatggaccgtagcctggcgggtctggtgccggatgaggaagttgacgcgctggttgagagcatccaccgtaacagcaagggtgaaagccaggaagcgaccggcgcggcggcgagcaacctggacctgtaccaagttaactgcacctactatgcggcgctgggtagcgacgatcagaaatatattgcggcgcgtgcggtgcagttctttatgccgggcgtgccgcaagtttactatgtgggtgcgctggcgggcagcaacgatatggacctgctgaagcgtaccaacagtggtcgtggcatcaaccgtcactactatagcgcggcggaagtggcgagcgaggtggaacgtccggtggttcaggcgctgaacgcgctgggccgtttccgtaacaccctgagcgcgttcgatggtgaatttagctacagcaacgcggacggcgttctgaccatgacctgggcggatgatgcgacccgtgcgaccctgacctttgcgccgaaggcgaacagcaacggtgcgagcgtggcgcgtctggagtggaccgatgcggcgggtgaacatgcgaccgacgatctgatcgcgaacccgccggtggttgcgggccaaactggccaccatcaccatcaccattagtaa

SEQ ID NO: 45 - protein, 264 aa, Artificial Sequence
  Description of Artificial Sequence Synthetic polypeptide
  uridine phosphorylase
MHHHHHHGGSGMSESDTFHMGLTKNDLQGATLAIVPGDPARVEKIAALMDNPVKLGSWREFITWRAELDGKPVIVCSTGIGGPSTSIAVEILAQLGIRTFLRIGTTGAIQGHINVGDVLVTTASVRLDGASGHFAPYEFPAVADFECTTALVEAAKSIGATTHVGVTASSDTFYPGQERYDTYSGRVVRRFQGSMEEWREMGVMNYEMESATLLTMCASQGLRAGMVAGVIVNRTQQELPNAESMKQVESHMVKIVVEAARRLL

SEQ ID NO: 46 - protein, 264 aa, Artificial Sequence
  Description of Artificial Sequence Synthetic polypeptide
  uridine phosphorylase
MHHHHHHGGSGMSESDTFHMGLTKNDLQGATLAILPGDPARVEKIAALMDNPVKLGSWREFITWRAELDGKPVIVCSTGIGGPSTSIAVEILAQLGIRTFLRIGTTGAIQGHINVGDVLVTTASVRLDGASGHFAPYEFPAVADFECTTALVEAAKSIGATTHVGVTASSDTFYPGQERYDTYSGRVVRRFQGSMEEWRVMGVMNYEMESATLLTMCASQGLRAGMVAGVIVNRTQQELPNAEDMPQVESHMVKIVVEAARRLL

SEQ ID NO: 47 - protein, 264 aa, Artificial Sequence
  Description of Artificial Sequence Synthetic polypeptide
  uridine phosphorylase
MHHHHHHGGSGMSESDTFHMGLTKNDLQGATLAILPGDPARVEKIAALMDNPVKLGQWREFITWRAELDGKPVIVCSTGIGGPSTSIAVEILAQLGIRTFLRIGTTGAIQGHINVGDVLVTTASVRLDGASGHFAPYEFPAVADFECTTALVEAAKSCGATTHVGVTASSDTFYPGQERYDTYSGRVVRRFRGSMEEWRVMGVMNYEMESATLLTMCASQGLRAGMVAGVIVNRTQQELPNAESMPQVESFMVKIVVEAARRLL

SEQ ID NO: 48 - protein, 264 aa, Artificial Sequence
  Description of Artificial Sequence Synthetic polypeptide
  uridine phosphorylase
MHHHHHHGGSGMSESDSFHMGLTKNDLQGATLAIVPGDPARVEKIAALMDNPVKLGQWREFITWRAELDGKPVIVCSTGIGGPSTSIAVEILAQLGIRTFLRIGTTGAIQGHINVGDVLVTTASVRLDGASGHFAPMEFPAVADFECTTALVEAAKSCGATTHVGVTASSDTFYPGQERYDTYSGRVVRRFQGSMEEWREMGVMNYEMESATLLTMCASQGLRAGMVAGVIVNRTQQELPNAESMKQVESHMVKIVVEAARRLL

SEQ ID NO: 49 - protein, 264 aa, Artificial Sequence
  Description of Artificial Sequence Synthetic polypeptide
  uridine phosphorylase
MHHHHHHGGSGMSESDSFHMGLTKNDLQGATLAIVPGDPARVEKIAALMDNPVKLGSWREFITWRAELDGKPVIVCSTGIGGPSTSIAVEILAQLGIRTFLRIGTTGAIQGHIGVGDVLVTTASVRLDGASGHFAPYEFPAVADFECTTALVEAAKSCGATTHVGVTASSDTFYPGQERYDTYSGRVVRRFRGSMEEWREMGVMNYEMESATLLTMCAVQGLRAGMVAGVIVNRTQQELPNAEDMKQVESHMVKIVVEAARRLL

SEQ ID NO: 50 - protein, 264 aa, Artificial Sequence
  Description of Artificial Sequence Synthetic polypeptide
  uridine phosphorylase
MHHHHHHGGSGMSESDSFHMGLTKNDLQGATLAILPGDPARVEKIAALMDNPVKLGSWREFITWRAELDGKPVIVCSTGIGGPSTSIAVEILAQLGIRTFLRIGTTGAIQGHIGVGDVLVTTASVRLDGASGHFAPYEFPAVADFECTTALVEAAKSIGATTHVGVTASSDTFYPGQERYDTYSGRVVRRFQGSMEEWREMGVMNYEMESATLLTMCASQGLRAGMVAGVIVNRTQQELPNAEDMPQVESHMVKIVVEAARRLL

SEQ ID NO: 51 - protein, 264 aa, Artificial Sequence
  Description of Artificial Sequence Synthetic polypeptide
  uridine phosphorylase
MHHHHHHGGSGMSESDSFHMGLTKNDLQGATLAIVPGDPARVEKIAALMDNPVKLGSWREFITWRAELDGKPVIVCSTGIGGPSTSIAVEILAQLGIRTFLRIGTTGAIQGHINVGDVLVTTASVRLDGASGHFAPMEFPAVADFECTTALVEAAKSIGATTHVGVTASSDTFYPGQERYDTYSGRVVRRFRGSMEEWREMGVMNYEMESATLLTMCASQGLRAGMVAGVIVNRTQQELPEAEDMPQVESHMVKIVVEAARRLL

SEQ ID NO: 52 - protein, 516 aa, Corynebacterium glutamicum
  wild-type catalase protein sequence
MSEKSAADQIVDRGMRPKLSGNTTRHNGAPVPSENISATAGPQGPNVLNDIHLIEKLAHFNRENVPERIPHAKGHGAFGELHITEDVSEYTKADLFQPGKVTPLAVRFSTVAGEQGSPDTWRDVHGFALRFYTEEGNYDIVGNNTPTFFLRDGMKFPDFIHSQKRLNKNGLRDADMQWDFWTRAPESAHQVTYLMGDRGTPKTSRHQDGFGSHTFQWINAEGKPVWVKYHFKTRQGWDCFTDAEAAKVAGENADYQREDLYNAIENGDFPIWDVKVQIMPFEDAENYRWNPFDLTKTWSQKDYPLIPVGYFILNRNPRNFFAQIEQIALDPGNIVPGVGLSPDRMLQARIFAYADQQRYRIGANYRDLPVNRPINEVNTYSREGSMQYIFDAEGEPSYSPNRYDKGAGYLDNGTDSSSNHTSYGQADDIYVNPDPHGTDLVRAAYVKHQDDDDFIQPGILYREVLDEGEKERLADNISNAMQGISEATEPRVYDYWNNVDENLGARVKELYLQKKA

SEQ ID NO: 53 - DNA, 1551 bp, Artificial Sequence
  Description of Artificial Sequence Synthetic polynucleotide
  catalase DNA sequence
atgagcgaaaagagcgcggcggaccaaattgttgatcgtggtatgcgtccgaaactgagcggcaacaccacccgtcacaacggtgcgccggttccgagcgaaaacatcagcgcgaccgcgggtccgcagggtccgaacgtgctgaacgacatccacctgattgagaagctggcgcacttcaaccgtgaaaacgttccggagcgtattccgcacgcgaaaggtcacggcgcgtttggtgaactgcacatcaccgaagacgtgagcgagtacaccaaggcggacctgttccaaccgggtaaagtgaccccgctggcggttcgttttagcaccgttgcgggcgagcaaggtagcccggacacctggcgtgatgttcacggtttcgcgctgcgtttttacaccgaggaaggtaactacgatattgtgggcaacaacaccccgaccttctttctgcgtgacggtatgaagttcccggattttatccacagccagaagcgtctgaacaaaaacggcctgcgtgacgcggatatgcagtgggacttttggacccgtgcgccggaaagcgcgcaccaagttacctatctgatgggtgaccgtggtaccccgaagaccagccgtcaccaggatggtttcggcagccacacctttcaatggatcaacgcggagggcaaaccggtgtgggttaagtaccacttcaaaacccgtcagggttgggactgctttaccgatgcggaagcggcgaaggtggcgggcgagaacgcggactaccaacgtgaagatctgtataacgcgatcgagaacggtgacttcccgatttgggatgtgaaagttcagatcatgccgttcgaagatgcggagaactaccgttggaacccgtttgacctgaccaagacctggagccaaaaagattatccgctgatcccggttggttactttattctgaaccgtaacccgcgtaacttctttgcgcagatcgaacaaattgcgctggacccgggcaacattgtgccgggtgttggtctgagcccggaccgtatgctgcaggcgcgtattttcgcgtacgcggatcagcaacgttatcgtatcggtgcgaactaccgtgacctgccggttaaccgtccgattaacgaagtgaacacctatagccgtgagggcagcatgcaatacatctttgatgcggagggtgaaccgagctacagcccgaaccgttatgacaagggtgcgggctatctggacaacggcaccgacagcagcagcaaccacaccagctatggtcaggcggacgatatctacgttaacccggacccgcacggtaccgatctggttcgtgcggcgtatgtgaagcaccaggacgatgacgatttcatccaaccgggcattctgtaccgtgaggtgctggacgagggtgaaaaagagcgtctggcggataacattagcaacgcgatgcaaggtatcagcgaagcgaccgagccgcgtgtttacgactattggaacaacgtggatgaaaacctgggcgcgcgtgtgaaagagctgtacctgcagaagaaagcgtaa

SEQ ID NO: 54 - DNA, 1551 bp, Artificial Sequence
  Description of Artificial Sequence Synthetic polynucleotide
  catalase DNA sequence
atgtccgaaaaatcagcggcggatcagattgtagaccgcggaatgcgtccgaaactgtccggaaacaccacccgccacaacggagcgccggtgccgtccgagaacattagcgcgaccgcgggcccgcagggcccgaacgtgctcaatgatatccatctcatcgaaaaactcgcgcactttaaccgcgaaaacgtgccggagcgtattcctcatgcgaaaggccacggcgcattcggtgaactgcatattaccgaggatgtaagcgaatacaccaaagcggatctgttccagcctggtaaagttacccccctggcagtgcgcttcagcacagtggcaggtgaacagggcagcccagacacctggcgcgatgttcacggcttcgcactgcgcttctacaccgaagagggcaactacgatattgtaggtaacaacaccccgaccttcttcctgcgtgatggcatgaaattccccgatttcattcattcacagaaacgtctcaacaaaaacggtctgcgcgacgcggatatgcagtgggacttctggacccgcgcgcctgaatccgcgcaccaggtaacctacctgatgggtgatcgcggtacccctaaaaccagccgccatcaggatggcttcggcagccacaccttccagtggattaacgcagaaggtaaaccggtgtgggtgaaataccatttcaaaacccgccagggctgggactgcttcaccgacgcggaagcggcgaaagtggcgggcgaaaacgcagattaccagcgcgaagatctctacaacgcaatcgaaaacggcgatttcccgatttgggatgttaaagtgcagattatgcctttcgaggacgcggaaaactaccgctggaacccgttcgacctgaccaaaacctggtcccagaaagactacccgctgattccggttggttacttcattctgaaccgcaacccgcgcaacttcttcgcacagattgagcagatcgcgctggacccgggcaacattgtgcctggcgttggcctgagcccggatcgcatgctccaggcgcgtattttcgcgtacgcggatcagcagcgttaccgcattggcgcaaactaccgcgatctgccggtaaaccgtccgattaacgaagttaacacctactcacgcgaaggtagcatgcagtacattttcgatgcagagggcgaacctagctactcacctaaccgctacgataaaggcgcgggctacctggataacggtactgacagcagcagcaaccacaccagctacggccaggcagacgatatttacgttaacccggatccgcacggcaccgatctggtgcgtgctgcatacgttaaacaccaggacgacgatgatttcattcagccgggcattctataccgcgaggttctggacgaaggcgagaaagaacgattggcggataacattagcaacgctatgcagggcatttccgaggcaaccgaaccgcgcgtttacgattactggaacaacgtggacgagaacctcggcgcacgcgttaaagaactgtacctccagaaaaaagcttaa